SEQUENCE LISTING (42 sequences)

SEQ ID NO: 1 - DNA, 195 bp, Bacillus subtilis Phage SP01
  promoter (1)..(195): The P15 promoter from Bacillus subtilis phage SP01, with a stem and loop added just downstream from the transcription start site.
gctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgtt

SEQ ID NO: 2 - DNA, 164 bp, Bacillus subtilis phage SP01
  promoter (1)..(164): The P26 promoter from Bacillus subtilis phage SP01
gcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgct

SEQ ID NO: 3 - DNA, 91 bp, Escherichia coli Phage
  promoter (1)..(91): The PR promoter from Escherichia coli phage
acgttaaatctatcaccgcaagggataaatatctaacaccgtgcgtgttgactattttacctctggcggtgataatggttgcatgtacaag

SEQ ID NO: 4 - protein, 359 aa, Neurospora crassa
  PEPTIDE (1)..(359): Protein sequence of  3-dehydroshikimate dehydratase from Neurospora crassa encoded by the qa-4 gene.
MPSKLAISSMSLGRCFAGHSLDSKLDAAQRYGYLGIELFYEDLVDVAEHLSNERPSPEGPFVEAQIAAARHILQMCQARGLEVVCLQPFMHYDGLNDRAEHERRLEKLALWIELAHELHTDIIQIPANFLPANQVSDNLDLIVSDLCKVADIGAQALPPIRFAYESLCWSTRVDLWERCWDIVQRVDRPNFGICLDTFNILGRIYADPTSPSGRTPNAKEAVRKSIANLVSRVDVSKVFYVQVVDAERLSKPLLPGHPYYNPEQPARMSWSRNCRLFYGETEYGAYLPVKEVARALFHGIGFEGWVSLELFNRRMSEEGPEVPEELAMRGAISWAKLVQDLRIPVEGPLVTMPRVSASL

SEQ ID NO: 5 - DNA, 2160 bp, Neurospora crassa
  gene (1)..(2160): Genomic DNA sequence of the qa-4 gene from Neurospora crassa plus surrounding sequences.
gaattcgggaaatggaatcttacctgggaaccgaaatcacagtccgggtaggttatagagcatatagtgaactgtcaaagttctagacctggaccagccacttggagtcgttgttttagttatacctacattcactcactgttgactttcaatcatacttacttagacggagcaacgcgccagaatccaaattgttgcatagttgcggtatcaccaagtggcttcccataatagtttgccattcgatgagacagctaactggaagaccggtactcgcaggttgcacgattacacggaaggattcggtattccgtgtttcatctgtcaaagtccctttccatatgaatccgaggtactatgactggatctcgatacaagctggccagcgaggtgcctgccttgacaggctgtcaactgcgggacggccggctaagtgtttaacacgcaagggtggaagatgtctcgtcccgtcatccaagaccgtcaacattcgaggccatctgatcgttgaagagatgctaaatcttgtgaaacgctcataggtcgcttaccttcggcccacccgttaatgctttattccgctgagcaaacttcggcttccatcccgcggttcaccgtttacatcacttatcgttgcggttattggccgattcttcgcaaaccgaaacgatgacatcccgaatatctgcaatacaccgccacggccggcgtctttatcacacctcctatgggagacgaaagtgccttgatacccctagtcatttgaagattcaggatgggagacggctggccgcttgcggagttacgttcgagtcttggtcgcaggaacgcttgccgtattgaatgagaccccgagaaggtcaaatcaaatcttggaagaccccaactgcttcctcattgccttcactccccatatcaatggggcacatcctgtgactaccttggtgctttatttcctcaccatttggcgatacaagctcaaggacaccgaggtgatatacagttcttcaaggacactatctcacctcaatatcaagaaccagtctcatcatctcttatttctccaggatccccccaccaacaacatcggcttttttttttcccctattctcaagacccatcaagacgctcacttcgctgagcctttcgccatgccgtcaaagctagccattagttccatgtccctagggcgctgctttgccggccactctctggacagcaagcttgatgccgctcaacgatacggctatcttggtatcgagcttttttatgaggatctggtcgacgttgcagagcatttgtcgaacgagcgtccctctcccgaaggcccttttgtcgaagctcagatagccgccgctcgtcatattctccagatgtgtcaagccagggggcttgaggtcgtctgcctccagcctttcatgcactacgacggccttaacgacagggcagaacatgagcgtcgtctggagaagctagcactatggattgagctcgctcatgagcttcacaccgacatcattcagatcccagccaacttcctccctgccaaccaagtcagtgacaacctcgacctgattgtctcagatctttgcaaggtggccgatattggagctcaagctttgccccctatccgctttgcctacgagagtctttgctggagcacccgtgtcgacctctgggagcgctgctgggacatcgtacaacgcgttgaccgccccaactttggcatttgccttgacaccttcaacatcctcggccgcatctatgccgaccctacatctcctagcggtaggacacccaacgcaaaagaggcagtcaggaagtccatcgccaacttggtctcgcgcgtggatgtctccaaagtcttctacgtccaggtggttgacgccgagaggctgagcaagccactactgcccggtcacccgtattacaatccagagcagccggcgaggatgagctggtcgcgcaattgtagactgttctacggcgaaacagaatatggtgcgtatcttcccgtgaaggaggttgctcgagcccttttccacggcattggtttcgagggctgggtcagtttggagcttttcaaccgcagaatgtctgaggagggacctgaagtgccggaggaacttgccatgagaggcgctatctcgtgggccaagt

SEQ ID NO: 6 - protein, 348 aa, Aspergillus nidulans
  PEPTIDE (1)..(348): Protein sequence of  3-dehydroshikimate dehydratase from Aspergillus nidulans. encoded by the qutC gene
MPANLKIGIPTVSLSKPGLHSLDHKLRSAAHGFAGIELFIDDLSHFASSSFNGSLTQAAKYISSLAKQLNLTFICLQPFGFYEGLVDTNQSTYLLTEKLPLWFAIARIIGTDLIQIPANFLQNDPVTGAARTSGDIRLIVSDLQTIADIGVKQGFRFVYEALCWSTHVDTWEAAWNVVKLVDRENFGICLDSFNTRTPLPSLGRRRMLSKPWPSPWRRSVLSSPVENWTSGKSSTSSLSMASGCRRRWTRSTPFMWRANPRRMSWSRNARLFPCEEERGGYLPVLEIARAFFEIGFEGWVSLELFSRTCNDPDVNTVGEHARRGMDRRRRVVAALGLDVEVPARNCEC

SEQ ID NO: 7 - DNA, 3298 bp, Aspergillus nidulans
  gene (1)..(3298): Genomic DNA sequence of the qutC gene from Aspergillus nidulans plus surrounding sequences
aagcttggtttcaagtgatgatatatagttatgaggatataatatgaaccgaaagacgatgtttcttgtgaatatttacgtgatagttgtctgtctaatatggtacagcagtagaacaactacatacggtcactacttacagccctagtcattccctccctcgattgcctaccatttatacactttgaacatccacaggcttgcctccctccatactctccctaacagcttgaacaactctgagcgccctcaccccatcttcaacgccacagccaactcctcgctcgccatcctcaccctttccactaacaacatcaacaaaatatccccactgcgcatcaaacggcctcacatcagcatccttcactgaaatctgctgcatcgccaactccgtattccagcctttctctttcccctgtccacaagaaacatggtcatagctccagcgcgtcatatcaggcacactgagactcgctctggttccaagaattcgataacagtcgcttgcactcttggaaggagcgggcggaatcgtaggattctcgcccgttcctgtttcaaagttcaacggcgaaggcgtcgcgtcgcagatgagaaatgtgcctactatcccagacgcaaagcgcagcgtcacagcacagccttcctcggcggtatgttccgggttctggcgcatgcgctgcaggagtgtaccctccgcgtagaccctactgacgggcccaaacagaaactgcagcacgtcgatatcgtggataagattaatccccagcacgccgcccttcttcttatctgcgcgccaagaaccgagcggcggcgcgaagtaagaggccggcttcagaagtgtccagaggccgttcactgcaacgacggtgccgagtgagtctgtctctaacaaagactttgtggtttggatgtacggattgaagcggcggtggtggccgatctggatgttgatcttcgcatccttgcctctcttctcatctttacatttctgttccttgacggtagcgaggaggtgctcggccgactccagatcgtcactgatcggtttctcaaggaggatattgcggattccgttctccagcagctggagcgtgacgtccacgtgcgtgtgattgggcgtgctcacgatcgccgcgtctggtttcccggttgtcttaccgacaacgtctaacatagacgtaatagaatcatagcaaggaacgccaaatgattctgcgaccgggattgcagagggtgaagggtcaacaaaagcgatcagctgggttcgtgggtgtcgttgcacggattgtgcgtgacggggcccaataagtccggcaccgacgatgacaatgaggatattcttgtccttttccttgctgcagggcaccattgtgcatgtcggtggctggaaataaacagaacagggatatggtcaagtcggagaatcggtgcaggatagaccggctacttgatgtaggacgacagtcgcgatctaccgagagcgtgagattcactgtgggactgatttatgtaatttgaggcgcagcagacttagggacttgaaatgtggctgtctgtggatgcatttgcggggtatggagtacagagtgcatacagctgtgtatatggagttccttacggagagggtgacctggtatggggagaacgggcaaaatgctcacccggcaacctctcaaagcgtttacccggtatactcctctgatatcaatatttccaatcagcacctatatcatcacgacgctctcctgaggattccgtagctaaccgccctggatcctacattaataaataagccatttgctttttctgctgcgagtgtgattctcaatacgattacgtatcacatgcagattgcctttacttcagctgcatttgatcagccacagctctaagagcaaacataccctacctacctacctacttcgcctagggtacataatcaccgccatctcctcctcgatcagtcttcaactcaatcagctcattcattctattcttaatataatatatacctttagatctccagcagagacccgaagagtcggcaattcaaaatgcccgcaaacctcaaaatcggtatcccaaccgtgtccctgtcaaaaccgggcctgcactctcttgaccataagctccgctcggccgctcatggcttcgcggggatcgagctgtttattgatgacctctcccatttcgcctcatcgtcattcaatggctccctcactcaagcggcaaagtatatctcctcgctcgccaagcaacttaacctcacatttatctgcctgcaaccattcggtttctacgagggtctggtggacacaaatcagtcgacgtacctgctcactgagaaactcccgctctggtttgcgatcgcccgcattataggcacagatctcatccaaatccccgcaaatttcctccagaatgaccctgtcaccggggctgcacgaacaagcggcgacataaggcttatcgtctcagatctgcagacgatcgcagatatcggtgtaaagcagggcttccgctttgtgtacgaggcgctctgctggtcgacgcatgtcgatacatgggaagcagcgtggaatgtcgtcaagctggttgatagagagaatttcgggatctgcctggatagcttcaacacgcggaccccgcttccgtcactgggaagacgccggatgctgagcaagccgtggccaagtccatggagacgctccgttctctcgtctccagtggagaactggacatcaggaaaatcttctacatccagcttgtcgatggcgagcggttgtcggcgccgctggacgagaagcacccctttcatgtggagggccaacccccgaagaatgagctggagtcgcaatgcgcggttattcccctgtgaagaggagaggggtgggtatcttcctgtgttggagatcgcgagggcgttctttgaaatcgggttcgaggggtgggtgagtctagagctgttttcaaggacgtgtaatgatcccgatgtgaacacggtgggggagcatgcgagacgtgggatggatagaaggaggagggttgttgcggcgctaggactcgatgttgaggtgccagcacgtaactgtgaatgttagcatgaacggcaaggagagggtggaggtgcaggtgcaggaggagctggctgttcagcatcggctgtaggtagtggtatcttgaaaggacgatagggtttgatctagagatttttattttgtctaattactggtaatgatggcctcatgcacgctgttgaacacgctgtacaacatcactgttgaagatgatacct

SEQ ID NO: 8 - protein, 502 aa, Klebsiella pneumoniae
  PEPTIDE (1)..(502): Protein sequence of protocatechuate decarboxylase (AroY) from Klebsiella pnemoniae ATCC25597
MTAPIQDLRDAIALLQQHDNQYLETDHPVDPNAELAGVYRHIGAGGTVKRPTRIGPAMMFNNIKGYPHSRILVGMHASRQRAALLLGCEASQLALEVGKAVKKPVAPVVVPASSAPCQEQIFLADDPDFDLRTLLPAHTNTPIDAGPFFCLGLALASDPVDASLTDVTIHRLCVQGRDELSMFLAAGRHIEVFRQKAEAAGKPLPITINMGLDPAIYIGACFEAPTTPFGYNELGVAGALRQRPVELVQGVSVPEKAIARAEIVIEGELLPGVRVREDQHTNSGHAMPEFPGYCGGANPSLPVIKVKAVTMRNNAILQTLVGPGEEHTTLAGLPTEASIWNAVEAAIPGFLQNVYAHTAGGGKFLGILQVKKRQPADEGRQGQAALLALATYSELKNIILVDEDVDIFDSDDILWAMTTRMQGDVSITTIPGIRGHQLDPSQTPEYSPSIRGNGISCKTIFDCTVPWALKSHFERAPFADVDPRPFAPEYFARLEKNQGSAK

SEQ ID NO: 9 - DNA, 5502 bp, Klebsiella pneumoniae
  gene (1)..(5502): DNA sequence of the aroY gene of Klebsiella pneumoniae 342 plus 2 kilobases of surrounding DNA sequences
gcgacgccgactgggcgatccgtgaactgctggcgcgtatgacccagcgtctgcagggctgtgaaaccatagaggatgtgattaaggtggcggagctgttcgcgccgaacatcgccccgacgatccccggtaaactgtatattctggataccgatccatggcagatgcgctgcgtggcgcagtggctgtcgcccgccggggagacgacgtcctttgctcccgacgactgctgggcgatacggcggggactcagccatccgccggtgcagggtgagcccgatatcacctgctatcatctgccggaggcgcacgccggccagtcgctctgcgtaccgctcatcgcccagggcgaagcgatcggtctgctgagctttcagaacgtcaccgccagtgacgccccttcccgggcttacctggagctgatggccgaagcgctggggctggcgctcgccaatcagcgtttacgcagcgccctgctggaaaaagcgttgttcgattcgctgaccggcctgcgtaaccgccatcatcttgatgaagcgctgcactcgcagatggcgctggcggtccatacccacaccccgctgagctgcctgatgatcgacatcgatcacttcaaagccatcaatgaccgctacggccatgaagccggggatctggtgattaagagcgtcgcgaccattgtgcagcgcgcggtgcgcgatatcggcatggctttccgctacggcggcgaggagtttttagtgctgctccccgggattgacgaagccggggcgcaccagtgcgccagcgagatctacacccaggtgcacaatatgacgctgcgcgatggcctgacggagataggccaggtggatgtgtcgattggcatcgccagctacccgcagcacacccaaagcgacagcctgctgcgcgcagcggacgccgcgctgtaccgggcgaaagagctgggccgttcaaggattgtcagctttggccgcctgaagacccgctaagcgggattattgctcagcggcattaagcagcgagataactttccgcaccaccgccgaacggaaatggcggtggtaaaccatgctcagctcactctccgccagccgatcctgaagatcgatatacaccacgttatcaaggcgtaaggccctcgccgagaccggcaccagcgccacccccaccccggcggagaccaggctaatcatcgaggtgacatcgttaattcgctgcaccacctgcggcgtaaaacccgcgacgcgacaggcgtcaataaatacctgctccagtccggtgccctgcggatcgtcaagcgagatccagttgtcagtgcgcaacgaggccagattgagcgcccccacgcctgccagcggatgctgttgataaagcgccaggcaaagtttttcccgcacaaatggcctgaccaccagcgcgtccggcggtgacgccagcggcgcgcggatgatggcgatatccagacgcagatccagcagcgcttcgtagagcatttgcacatccccctgcaccagcgacagctcaatccccggccagtcagcgcgcagctcgcgcaggagccccggcagtttgctgtcatacatcgcactggagacatagcccagatgcaatcgcccctgctcgcctcgtgcggtgcgctgggcgtccaggaccgcctgatcggccatctccagcgccagccgcgtcttctgcaagaaggcctcgcccgcggcggtgagggtcaggcgccggttagcgcgggagaagagcaccacgcccaggcgctgctcgagttgtttaatctgctggctgagggcgggctgggcgatatgtaaccgctctgccgcccgatgcatatgtagttcttcagcaacgaccacaaaatggcgtaacgctcgcaaggacatggccggactccgcggagtaaattgataataaaaatgttatcaataaagcatgaatgatgcaattgataaccattagcctgcgagcatactgtgcgcatcgacacgctaaggagaacatcatgaccgcaccgattcaggatctgcgcgacgctatcgcgctgctgcaacagcatgacaatcagtacctcgaaaccgatcatccggttgaccctaacgctgagctggccggcgtctatcgccacatcggcgcgggcggcaccgtgaagcgccccacgcgcatcggcccggcgatgatgtttaacaatattaagggctatccgcactcgcgcattctggtgggcatgcacgccagccgccagcgggccgcgctgctgctgggctgcgaagcctcacagctggcgctggaggtaggcaaagcggtgaaaaaaccggtcgcgccggtggtcgttccggccagcagcgccccctgtcaggaacaggtctttctggccgacgatccggattttgatttgcgcaccctgctcccggcgcccaccaacaccccgatcgacgccggtcccttcttctgcctgggcctggcgctggccagcgatcccgacgacgcctcgctcaccgacgtcaccatccaccgcttgtgcgtccagggccgggatgagctgtcgatgtttctcgccgctggccgccatatcgaagtgtttcgtcagaaagccgaggccgctggcaaaccgctgccgataaccatcaatatgggactcgatccggctatctatatcggcgcctgctttgaagcgccgaccacacgtttggctataacgaactgggcgtcgccggtgcgctgcgtcagcgtccggtagagctggtacagggcgtcagcgtcccggagaaagccatcgctcgcgccgagatcgttatcgaaggggaactgctgccgggggtacgcgtcagagaagatcagcacacaacagcggccatgcgatgccggaatttcctggttactgcggcggcgccaatccgtcgctgccggtcattaaagtcaaagcggtgaccatgcgaaacaatgcgattctgcagacgctggtagggccgggcgaagagcataccaccctcgccggattgccaacggaagccagtatctggaatgctgtcgaggctgctatcccgggctttttacaaaatgtctacgcccacaccgcgggcggcggtaaattcctcgggatcctgcaggtgaaaaaacgccagcccgccgacgaagggcgtcaggggcaggccgcgttgctggcgctggcgacctattccgagctgaaaaatatcattctggtcgatgaagatgtcgatatctttgacagcgacgatatcctgtgggccatgaccacccgcatgcagggggatgtcagcatcacgacgatcccaggcattcgtggtcaccagctggatccttcccagaccccggcctacagcccgtcgatccgcggagagggtatcagttgcaagacgattttcgattgcacggtgccgtgggcgctaaaatcacacttcgagcgcgcaccgtttgccgatgtcgatccgcgtccgtttgcgccggagtattttgcccggctggaaaaaaaccacggtcagtaaaatcaggtgatagccgccggagcacggcggcatcttccgggccagcatcacctgcagcgggtggctgacgcagggttagttgatcgcggcggagaggtcttttttcacctgctcacgctgctcgggggtcaacacctggctcacgtcgaagtagtatttcacacgataataccgcacctgctggtccagctggccaaaggcggccagctgctgtttgaccttagcgtcatcccatttcccggagtgaataacgtctgccagggcaccatcctgatagccgctgatttaatctggcttacattgttttcgaatccctgacgcagcgcctggattttggcgacctgctcttcactcagcttcaggtgctggacgaccggatcctgcgagacagacggtatatcggcggaggtcgacgcctggctggctgccgtaaagcaggtggtcagcgcaatggcgagcagggtgttacgcaagcgagtattcacagtgaatgatccttcaaaaaagaaaatgagaggcgattatcactgcgctaataaagactatctgtaacaaagggttaatttaaaactggataaaaaaaggatggtaagaaacagaaatcagatcccgggtcagcagcacagaaagatatattcatccttccagtaacggccctgtccaatgatatccccggcggcgctgattaactgtttttgcttttggtttcaatcccctcaacgatcacatggctggtcagggtatgaatagattgcaacagcccgggaaaagcggggtcgttttctttatcccagaagtaatctttatccactttgacgcaatcgaagcggaagcgctccagcagcgggaaggacgtcgtccgcggccaaaatcatccagccagaccgggcaaagcgccgccagcgtgctcagcgccgtcagctcacgtccggcgataaactcgtgaaagttctcatttatttccagagcaatgtgtttacaggagcgcaggaaatcacagagatagcgatccgtcagaataaagtggcttaataaatcatcaatattcagcgatatcggtttattatcgacccgggcgaaatcaaatacagaaagtaactcgatttgccgaataaatagagcaagcttatcgcgttcggtaagcgtggagaaagaaaacgtcgacgcagaggtggtattttgtgcgggtgctataatatctttagtaagcaactcccacgagtggtagctgccgtcatcgctaatagcgggctccagaacgaagcgataagaggtatttttcacgttttcttcaaccatttaaaaaataccaaaaataagaaagggttaagcatgtcatatattttccgccaacaaaaatagtttaaagtgatcgataataatcattcgatagttaaaaactatcaagatataatttattgatcggtaaattgaattaatataaattagccactgccgtaactccctctgaaaagtcaattaaaatattgtttcaaaccagccagttaccagagtattctgcgtaaagcctggtcgtctcacgctttgtgctgccaggtaaaaaaagagaggggtaataaaaatgaaaaatacaagccgccagttttagtcatatcattatgccgaatatgaataacgctgcgctgaggcgccgcttcgcctggcatgccatgagtcctcaacaaaaaagtgtgactcagtcgacaaaacgtcatattttcccgctatcctgcagcgaagaagagtgaagtggatgacaggcagtgaaaaaaataaacgtgattccgctggggctgatgctattcatgctcatcgccagcgcatggctgggccctgcgccgcggcacaccggcagcatgcagtgcgtttggtttgacggggcaatggtgagctgcctgccgaagcaacgactgggcgaaggctcgccgcatcatttactggtcagacgataaaccggtactcgccgggtggtgttgaacagattatcgctggc

SEQ ID NO: 10 - DNA, 4629 bp, Acinetobacter baylyi
  gene (1)..(4629): DNA sequence of the catA gene from Acinetobacter baylyi ADP1, including 410 bases of upstream sequence and two open reading frames downstream
atctgctcgaccatagtaatgatcacattatgagctaaatttactttttaaaatttaaatatattatatatatttgaattttattgttttattttaatttttagcttagaagtttttattaagatttatttttaaattagatgtcgaaaaaattagtataccaaaaaagcatgaaaacatactctcttaggaattggagtcgccatgagtttcagatacagttgatcagtatggaaggtatagaaacgactatcgaaataaataagtttgtggtgtgtgaagcaaggtaaagctcaaggctgaggcaaaccaagcaaaggttaattgaaccgatatgcacaacacattcaacgatagcgtcgacagataagtttatcaaatgatgttttggcgatttcaaggagaaagccatggaagttaaaatattcaatactcaggatgtgcaagattttttacgtgttgcaagcggacttgagcaagaaggtggcaatccgcgtgtaaagcagatcatccatcgtgtgctttcagatttatataaagccattgaagatttgaatatcacttcagatgaatactgggcaggtgtggcatatttaaatcagctaggtgccaatcaagaagctggtttactctcgccaggcttgggttttgaccattacctcgatatgcgtatggatgccgaagatgccgcactaggtattgaaaatgcgacaccacgtaccattgaaggcccgctatacgtggcaggtgcgcctgaatcggtaggttatgcgcgcatggatgacggaagtgatccaaatggtcataccctgattctacatggcacgatctttgatgcagatggaaaacctttacccaatgccaaagttgaaatctggcatgccaataccaaaggcttttattcacacttcgacccaacaggcgagcagcaggcgttcaatatgcgccgtagtattattaccgatgaaaacggtcagtatcgcgttcgtaccattttgcctgcgggttatggttgcccaccagaaggtccaacgcaacagttgctgaatcagttgggccgtcatggtaaccgccctgcgcacattcactattttgtttctgccgatggacaccgcaaactaactacgcaaattaatgtggctggcgatccgtacacctatgacgactttgcttatgcaacccgtgaaggcttggtggttgatgcagtggaacacaccgatcctgaagccattaaggccaatgatgttgaaggcccattcgctgaaatggttttcgatctaaaattgacgcgtttggttgatggtgtagataaccaagttgttgatcgtccacgtctagcggtgtaatacaccaaaatggttcaaaattatcaggcgagtgatcatgatcactggcctgtttttatttcagggaagggtggagacaattacgtggacaatcaaatcattcaggaaaccgtagataaaattttaagcgtattgccgaatcaggctgggcaattggcacgcttggttcgtctgatgcagtttgcttgtgaccccaccattaccgtcattggtaaatataatcatggtaaaagccgactactcaatgagctgatcgggacagatattttttctgttgccgataaacgagagacgattcaactggccgaacataaacaagatcaggtgcgttggttggatgcacccggactcgatgcagatgttgcggcagtggatgatcgtcatgcttttgaagcagtctggacacaggcagatattcgcctttttgtgcattcagtccgagaaggcgaactcgatgcaactgagcatcatcttttacaacaacttattgaagatgcagaccatagccggcgccaaaccatactggtcttgacccagatagatcagataccggatcagacaattttaacccagattaaaacctcaattgcacagcaggtacccaaactcgatatttgggctgtttcggccactcgccaccgtcagggtattgaaaatggaaaaaccttgctgatcgaaaaaagtggaatcggcgcgttacgacatacacttgagcaggcacttgctcaggttccatctgcacgaacgtatgaaaagaatagattgctgtctgacttgcatcatcaacttaagcagttattactcgatcaaaaacatgtacttcagcaactacaacagacacagcagcagcaattgcatgactttgatacaggactcatcaacatactcgataagattcgagtagatcttgagcccattgtaaatatagatggtcaagaccaagcactcaatccagattcatttgccacgatgtttaaaaatacagcagccaagcagcaacgtgccaaagtgcagattgcttactcacgtgcctgtatcgagatcaatagccatctcatacgtcacggtgtggtgggtttacccgcagagcaacaaaccacgataaaaagtattgatacggtcattgttgcggtttttggaatttcagtgaaatttcgcgatcagctacgtgcattgttttataccgataccgaacgacaacgcttgcaaagagagtttcgattttattttgaaaagtcagcaggccgaatgattttagctgccaagattgagcagacaatgcggcagcaagggtgtattcaaaatgcaatgatggcgttgcaacagatggagagtgcagcatgaccagcggcggacacattcaattgtttatcgaacacacccggcagattgcgactgcccaaggggatatacagttggcattgcaatcgatgcagcaatggcgcgaagcatttgctacagcattaaaacaaaatacctttgatttaacgggctggtcaccgcagacaaagatcgccaatcaactcaagcaatttaaccataagcttacaacgcatgtatcgaattgggataccgaatggcatacttttagtgctgctcaatcggttgcagaagtatttcatgatcgggtgatgttgcttgtattcggtaagtttaatgccggaaagagttcattgtgtaacttactggccgaatgctttcgttctcacgaacaaaccgtgcaatattttcatgttcaaaatgaacagatattttataccgaatctcacttacgcgaaggtgcaaccgagacgacagcgcaactacagggcgtatgtctgggtgaaaaacttattttgctagatacaccaggtttgcattctggtactcagaaaaatgcagcgctcacacaaaaatttatcgacagtgcagatggtgtgctgtggctcagtagcgcaacttcaccgggtcaggtgcaagagctagatgcactggggcgcgagttaaagcgtcataaacctttatttcctgttattacccgaagcgattttgtcgaagaagatgaaattgatggtgagctatgtacagtgctttgcaataaaaattcagaacaacgtgcgttgcaagagtctgatgtattgatgcgtgcgaaagaaaaactgcacatatgcaagtggatgtgagtttattaaagccgcccgtgtccgtttcaactcaaatggcgcgtgaagcagatatgaacccacaagccatgaacgaggctggttttgagcgattatttgcagcacttttggctcttattgagcctgctttgcgctataagcagcgtaaacctgccgaagtattgttgcattttttgcaagaacatatcattgaaggtttaaggttttacctgcaacccgatctagagcaaatacaacaggacctcaaacaggctcaagatgatttacgacagctacacaccgatttagccgaggcagtctggcgtagcgtattgcctgagctaccacaacttcttgagcaacatgcaagtacacaaaatattgatgccgtagtgaacagtttgaacgagtggataaacgtcgcattcgaacaacagcttgcaattcagcttgatgcttatggtttaaatttggattcgcttagcaagatcgaaaaaaccgaaaaaatgcagtatgaacgcattgcgggaatggtggtgcatgatggcttgtacacgactctcacgcagcagattcaacaagctgtcaaagcttctacgagtgaattgattgatcagtgtcaggctcaacttgagcagtcaatcaaacatgttcaaacactcgatgaaaccttcatcgattacagcgcagcactcgatcaactcagccaagcgctacgcattgaataaagagcagtaaatttttcagacatattttattcgatgagtggcctgatatggtgcgttgcaaacacctcctgtacacaggcgagaattttaggaatgtaattactgtggtccatatttcgcaccgcgagtgaaattgggctataggcatcatcatctaaaattggaatataaagtagattcttcaccccaatatccatggcagacgccggtacgatgcagacgccttcacctgctgccaccaagccgagtgccagttgaatttctcgaatttcggtgagtttggatggtactaggcctagttcggtaaagagtgactgaataaaggtcgcaaaattgggcttttgagagactgggtacagcagcatcggttcatcaataatttgagagagatgaacccctgttgctgcaaactga

SEQ ID NO: 11 - protein, 311 aa, Acinetobacter baylyi
  PEPTIDE (1)..(311): Protein sequence of CatA (catechol 1,2- dioxygenase) from Acinetobacter baylyi ADP1
MEVKIFNTQDVQDFLRVASGLEQEGGNPRVKQIIHRVLSDLYKAIEDLNITSDEYWAGVAYLNQLGANQEAGLLSPGLGFDHYLDMRMDAEDAALGIENATPRTIEGPLYVAGAPESVGYARMDDGSDPNGHTLILHGTIFDADGKPLPNAKVEIWHANTKGFYSHFDPTGEQQAFNMRRSIITDENGQYRVRTILPAGYGCPPEGPTQQLLNQLGRHGNRPAHIHYFVSADGHRKLTTQINVAGDPYTYDDFAYATREGLVVDAVEHTDPEAIKANDVEGPFAEMVFDLKLTRLVDGVDNQVVDRPRLAV

SEQ ID NO: 12 - DNA, 1461 bp, Acinetobacter sp. ADP1;
  gene (1)..(1461): DNA sequence of the quiC (3-dehydroshikimate dehydratase)gene from Acinetobacter sp. ADP1
atgaaattaacttctttacgcgtatctttattggcgctgggcttggtaacatcaggttttgctgcggcagaaacttatactgtagatcgttatcaggatgatagtgaaaaaggctctttgcgttgggcaattgaacaatctaatgcaaatagcgcacaagagaatcagattctgattcaggctgttggtaaggcaccttatgtgatcaaggtggataaaccgttaccaccgattaaatcatctgtaaaaattattggtacagaatgggataaaacgggcgaatttattgcgattgatggttcaaactatatcaagggcgaaggcgaaaaagcatgtccaggtgcaaatccaggacaatatggtaccaatgttcgtaccatgactttaccaggtttggttctacaagatgtcaatggtgtgaccctgaaaggtcttgatgttcatcgcttctgtattggtgtactggtaaatcgttcaagcaataatttgattcagcataaccgtatttcaaataattacggtggcgctggtgtcatgatcacgggtgatgatggtaaaggtaacccaacgtctaccaccaccaataacaacaaagtattggataatgtgtttattgacaatggcgatggtcttgaactgacgcgtggagcagcattcaacctgattgctaacaatctgtttacatcgaccaaagccaatccagagccgtctcaaggcattgaaattctttgggggaatgacaatgcagtggtgggtaacaaatttgaaaactattcagatggtctacaaatcaactggggtaaacgtaattacatcgcttataacgaattgaccaataactctttgggtttcaatcttacaggtgatggaaacatcttcgatagtaacaaagtgcatggcaatcgtattggtatcgcaattcgttctgaaaaagatgcaaatgcacgtatcacacttaccaaaaatcagatttgggataatggtaaagatatcaaacgctgtgaggctggtggttcatgtgttccaaaccaacgtttaggtgcaattgtatttggtgttcctgcgcttgagcatgaaggttttgtaggctctcgtggtggcggtgtagtcattgaacctgcaaaattacaaaaaacatgtacacagccaaatcaacaaaactgtaatgccattccgaaccaaggtattcaggcacctaaactgactgtcagtaaaaaacaacttacagttgaagttaaaggaacaccaaaccagcgttacaacgtagaattttttggaaatcgtaatgcatcttcttccgaagctgagcaatatttaggttcaattgttgtagtgacagatcatcaaggtcttgcaaaagcaaactgggcaccaaaagtcagcatgccatctgttactgcgaatgtaactgatcacttgggcgccacttcagagttaagttctgcagtgaaaatgagataa

SEQ ID NO: 13 - DNA, 1461 bp, Acinetobacter sp. ADP1
  CDS (1)..(1461): Codon-optimized DNA sequence of the quiC (3-dehydroshikimate dehydratase)gene from Acinetobacter sp. ADP1
atgaaactgaccagcctgcgtgttagcctgctggcactgggtctggttaccagcggttttgcagcagcagaaacctataccgttgatcgttatcaggatgatagcgaaaaaggtagcctgcgttgggcaattgaacagagcaatgcaaatagcgcacaagaaaaccagattctgattcaggcagttggtaaagcaccgtatgttatcaaagttgataaaccgctgcctccgattaaaagcagcgttaaaatcattggcaccgagtgggataaaaccggtgaatttattgcaattgatggcagcaactatatcaaaggcgaaggtgaaaaagcatgtccgggtgcaaatccgggtcagtatggcaccaatgttcgtaccatgaccctgcctggtctggttctgcaagatgttaatggtgttaccctgaaaggtctggatgttcatcgtttttgtattggtgttctggttaatcgcagcagcaataacctgattcagcataatcgtatcagcaacaattatggtggtgccggtgttatgattaccggtgatgatggtaaaggtaatccgaccagcaccaccaccaataataacaaagttctggataacgtgttcatcgataatggtgatggtctggaactgacccgtggtgcagcatttaatctgattgcaaataacctgtttaccagcacaaaagccaatccggaaccgagccagggtattgaaattctgtggggtaatgataatgccgtggtgggtaacaaattcgaaaactattcagatggcctgcaaatcaattggggtaaacgtaactatatcgcctataacgaactgaccaataacagcctgggtttcaatctgacaggtgatggtaacattttcgacagcaataaagtgcatggtaaccgtattggtattgccattcgtagtgaaaaagatgccaatgcacgtattaccctgaccaaaaatcagatttgggataacggcaaagatatcaaacgttgtgaagccggtggtagctgtgttccgaatcagcgtctgggtgcaattgtttttggtgttccggcactggaacatgaaggttttgttggtagccgtggcggtggtgttgttattgaaccggcaaaactgcaaaaaacctgcacccagccgaaccagcagaattgtaatgcaattcctaatcagggtattcaggcaccgaaactgacagttagcaaaaaacagctgaccgttgaagttaaaggcacccctaatcagcgttataatgtggaattttttggcaatcgtaatgccagcagcagcgaagcagaacagtatctgggtagcattgttgttgttaccgatcatcagggtctggcaaaagcaaattgggctccgaaagttagcatgccgagcgttaccgcaaatgtgacagatcatctgggtgcgaccagcgaactgagcagcgcagttaaaatgcgttaa

SEQ ID NO: 14 - protein, 486 aa, Acinetobacter sp. ADP1
MKLTSLRVSLLALGLVTSGFAAAETYTVDRYQDDSEKGSLRWAIEQSNANSAQENQILIQAVGKAPYVIKVDKPLPPIKSSVKIIGTEWDKTGEFIAIDGSNYIKGEGEKACPGANPGQYGTNVRTMTLPGLVLQDVNGVTLKGLDVHRFCIGVLVNRSSNNLIQHNRISNNYGGAGVMITGDDGKGNPTSTTTNNNKVLDNVFIDNGDGLELTRGAAFNLIANNLFTSTKANPEPSQGIEILWGNDNAVVGNKFENYSDGLQINWGKRNYIAYNELTNNSLGFNLTGDGNIFDSNKVHGNRIGIAIRSEKDANARITLTKNQIWDNGKDIKRCEAGGSCVPNQRLGAIVFGVPALEHEGFVGSRGGGVVIEPAKLQKTCTQPNQQNCNAIPNQGIQAPKLTVSKKQLTVEVKGTPNQRYNVEFFGNRNASSSEAEQYLGSIVVVTDHQGLAKANWAPKVSMPSVTANVTDHLGATSELSSAVKMR

SEQ ID NO: 15 - protein, 486 aa, Acinetobacter sp. ADP1; identical to SEQ ID NO: 14
  PEPTIDE (1)..(486): Protein sequence of QuiC (3-dehydroshikimate dehydrogenase from Acinetobacter sp. ADP1

SEQ ID NO: 16 - DNA, 9462 bp, Escherichia coli
  DNA sequence of the plasmid pAC21 (1)..(9462)
  misc_feature (5924)..(5924): n is a, c, g, or t
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccccccgccgccgacagagtaataggttttacttaatagctcttcctgtcccttccaggcagtgatccgcattccgttctcatggcgaggcaacatttcgggatggaagataatgttctttgctacaggaaaatcaacaatatgcgcaccagatgccactggcagccgcccgctgcgcgttactaactctataaatgcagggatctcatcaatgacaacatcctgcggactgtttcctgccagtcccatgatgatggcgacatccgtggcatggcctttgcccgtcagtgacaacgacccgtacagatcgaccacaatatggctcgtcgcggttaataagccgctactttccagccgatcaataaaactttttccggcattcattggccccacggtatgcgaactggagggaccaatcccaattttgaaaatatcgaatgcactaatcatgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttantcggtacccggggatcgcggccgcggaccggatcccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccaatatcctattggcattttcttttatttcttccatttaaatggatgcatgcgctagcggagtgtatactggcttactatgttggcactgatgagggtgtcagtgaagtgcttcagcctcgtgagcgggacggtcgtaaggtcgttccgctccacttcactgaacggcaatccgagggtgtggatccaattaaggccacgctgtcatttaaattccgtttttccagttcaaatgcaattgccttcaatgcaccttcgtagctgtggtgagccagcggtgctggctctcccccatttacggataagaatgcattttccgagttaataccgtcggcaatacctgacattaatacttcacagtcgctggcatcgagtacggaaaacttaatcgaagacgaaccacagttaataaccaaaacaaccggaaattcattcatctcttttctcatcctgagttacggattaaaacagtttgtatacgatgttcaggatggtcagcagaccaatcacggtaacaaacacgttatccagacgaccacggtatttcgccagagacggcgctttacggatggcatacatcggcaacaggcacagcagggatgcgataatcggtgcgcccatggcttcaatcaggtcgaggatgttcgggttggcgtaggcaacaacccaggtggagcccatgatgaagatcatgctgagagtattcagtttacccagcgacactttggttttgtcacctttataaccgaacttcagaatcagaccattcaagccttccagcgtccccagatagtgaccgaagaaagatttgaagatagccacgagtgcgatgatggaagccgcatattccagtgtaatcgcgaacgttgttttggtaccggtcatggacgcaaagtggttagccagataagaaagcactggaatattctgcgctttggcttccgccatgttggccggagacagagtaaacaggcagctaaaggcaaagaacatcaccactgcaaccatcagcatgctggcacgagaaatgatttgggaacatttacgttcggtgaagtcgcgaccgaagtctttctcatactcttcacgtttagaaaccacgaaggaagagacgattggcgagaagttaaaggagaaaaccatgatggaaatccccagccagacagtgatcaggataccgtcatgaccggttaacgacagcgaaccgaggtcaacctggtcgataactgcagagttccagtaagggatcagcgacaaagaaatcagcaccaggctggcgataaacggccataccaggtagctcagggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 17 - DNA, 9430 bp, Escherichai coli
  DNA sequence of the plasmid pAC19 (1)..(9430)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatcccccccgccgccgacagagtaataggttttacttaatagctcttcctgtcccttccaggcagtgatccgcattccgttctcatggcgaggcaacatttcgggatggaagataatgttctttgctacaggaaaatcaacaatatgcgcaccagatgccactggcagccgcccgctgcgcgttactaactctataaatgcagggatctcatcaatgacaacatcctgcggactgtttcctgccagtcccatgatgatggcgacatccgtggcatggcctttgcccgtcagtgacaacgacccgtacagatcgaccacaatatggctcgtcgcggttaataagccgctactttccagccgatcaataaaactttttccggcattcattggccccacggtatgcgaactggagggaccaatcccaattttgaaaatatcgaatgcactaatcatatccacaccctcggattgccgttcagtgaagtggagcggaacgaccttacgaccgtcccgctcacgaggctttacgcactacgtactgcgatggcttcaatttccagcgggagggcggatccactaatacaaaatatatcaaaagttaataataatattattcttacttaagacttttttgtcttcattttttagtaaaaaatataaaaaaggccacctcccgattttatcggaaggcagcctcttaaattcagttcataatattaaaaaatattattcaacttcagaatatttgttggcataggcagctgccgcacccaacagtccaggctgcggataagtaatcaacttaaccggaatcttggacatgacgcgttcaaagcgtccttttgaaacaaagcgctgacggaaaccagattctggcaaatgggaagcgatacgaagaccgacaccaccgccaataacaacactggttgcaccctgtgccaaagcaagatcaccagcgatagcgccaaggctcaagcagaagcgatccaaagcggcttcagcaaggttgtctttaccttccaaagccatctgccataatttaatatcatccagcaagctgaacggaacgccttcaatggcagccagtgcttcgtagatattaccaagacccgggccagaaataatgcgttcgatagaaacgcggcggaaacgttcacgtaaacgtgccagaattttgtcttcaagtctgtcaagcggagcaaagtcgatatgaccgccttcagtttcgatgacgaaataacggccttcagtccgcaacagatgggcaacacccaagcccgttcccggaccaagaatagtgataacaccatcgctaggaagcgcttcatcaggaccacaaatatgatccagataagaagaatccatatgcgcaaccgcgtgggcaaccgcgccgaagtcattgatcagaacatgcgtatcgatgtccagcttttcattcagagtagctggtcttaatacccaagggttattggtaagttttaaaacttcaccatgaaccgggccagcccatgcaatagctgcggcacgtggcagaggacgacccagtttttcaccgaaacgttcccaagctaactgcaagctagcatgttctgccgttttaaaagttgtttcttctccaagagaaagaacccgaccattgcttacttccgcaatagagaaacgcgcatgcgttccaccgatgtcaatcgcaacaatttccataataattcctttctgaaatcagaaggctacccaacaggtaaaataagtccgcccgctttataccatcgttgtaaacaaaaagtataattggttaagacttatctaaaaaagacaaaaggattcagccaaagcaagtttaactacttctgggagcgccacatctcctcgatttcatccaggctccgacctttggtttccggcacgaagcgagcaacaatcaagccacctaagatacttaatgctgcgaaaacgagataggagaaaccgtggttgaaagtctgattcaatgctggagaaccatcggcaaccttaaacaggaagttaaccaagatattagctaaccattgtccggtaacagcgataggcatagctgcgcccttgatggaactcgggaacatttctgacagaacaacccagcagacagggccccatgacataccaaagactgcaatataaagaagcacagaagccaaaggcaaaacaccaccgactttgaaccagaaacagcagcctaaaacagccatcattgcagccataccgagagcaccccaaataagcagaggtttacggccgaagcggtcaacaacacgggaagcaatcatggtgaagatgaagttcacaacaccgatagagatggtctgcaataatgccgtatcagctccaaaacctaaattctggaacatctgcggtgcataatacagcacggcgttaataccgactaactgctggaaggcagcaacggatacaccggcaaaaacaacggtgataccaaaagcaaacaaacctgcgctgcttttgtccatggctttatcaaagccagctttaatcttttgaatcgtcagattaggatcggcttgcggttccagacgagcaaggattttgctagcctcggaatgacgtcccttcatcaccaaccaatgcggcgtatccggtgcggttaacagcagcaataagaaggcaataccgatcaggccttctgaagccggagaccagcaccaaccactggcattaacccaatcgatagaaccgaaatgagccagtaaccaggtaaagatataaccggttaaagcacccgtcacaatggccatctgctgaccagaaaccatctgaccacgtttgtctggcggagcaatttcagcaatataggttggggtcaaggttgaaacgacaccgatacctaaaccggcaagaaaccggaaaaagcaaaaaatttgtaaagccgaaccaccggttccaaataatttttcggttaacgcagcaccaaaaccggcggcgacgaaacaaatggaactcatcaacaatccgccgcgacgaccgaagcgaataccaatccagccagacagcaaagaaccggtaacacaaccgaccaaaacagcaacaacgaccatcccagaaagggaagccgcagccgtagcagacaggtgacgaggggcaataaaatggatatcaaccggtgtaccgattgcagcgataaccgctgaatcgtaaccgaaaagcaagccgcctatagcagcgattagggctagtcgcgtgactagaccctgactactttcagaactcatggcgattcctctccctctagagcgtcctgctgttgttaagattattataccacaccttgtagataaagtcaacaactttttgcaaaatttttcaggaattttagcagaggttgttctggatgtagaacaaaacatctttccgctcttgtgctgttaggatatctttcttggaagctaggtaggcctcgagttatggcagttggttaaaaggaaacaaaaagaccgttttcacacaaaacggtctttttcgatttctttttacagtcacagccacttttgcaccaattaaggccacgctgtcatttaaactccgtttttccagttcaaatgcaattgccttcaatgcaccttcgtagctgtggtgagccagcggtgctggctctcccccatttacggataagaatgcattttccgagttaataccgtcggcaatacctgacattaatacttcacagtcgctggcatcgagtacggaaaacttaatcgaagacgaaccacagttaataaccaaaacaaccggaaattcattcatctcttttctcatcctgagttacggattaaaacagtttgtatacgatgttcaggatggtcagcagaccaatcacggtaacaaacacgttatccagacgaccacggtatttcgccagagacggcgctttacggatggcatacatcggcaacaggcacagcagggatgcgataatcggtgcgcccatggcttcaatcaggtcgaggatgttcgggttggcgtaggcaacaacccaggtggagcccatgatgaagatcatgctgagagtattcagtttacccagcgacactttggttttgtcacctttataaccgaacttcagaatcagaccattcaagccttccagcgtccccagatagtgaccgaagaaagatttgaagatagccacgagtgcgatgatggaagccgcatattccagtgtaatcgcgaacgttgttttggtaccggtcatggacgcaaagtggttagccagataagaaagcactggaatattctgcgctttggcttccgccatgttggccggagacagagtaaacaggcagctaaaggcaaagaacatcaccactgcaaccatcagcatgctggcacgagaaatgatttgggaacatttacgttcggtgaagtcgcgaccgaagtctttctcatactcttcacgtttagaaaccacgaaggaagagacgattggcgagaagttaaaggagaaaaccatgatggaaatccccagccagacagtgatcaggataccgtcatgaccggttaacgacagcgaaccgaggtcaacctggtcgataactgcagagttccagtaagggatcagcgacaaagaaatcagcaccaggctggcgataaacggccataccaggtagctcagggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 18 - DNA, 5768 bp, Eschericahi coli
  DNA sequence of the plasmid pMH17F (1)..(5768)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagttacctagagagggtgagaattgccgaacatgcgcataagtttcccggacagatttcaggtggtcagcagcaacgcgttgccattgcgcgttcgctgtgtatgaagccgaaaattatgttgtttgatgagccaacgtcggcgctcgatcctgagatggtgaaagaggtgctggatacgatgattgggctggcgcagtcgggtatgacaatgttgtgtgtaacacatgagatggggtttgcacgaaccgtcgctgaccgggtaatttttatggatcgtggggaaatagtggagcaagctgcacctgatgaattttttgcgcatcctaaatcagagcgtacgagggcatttttatcgcaggtaatccattaattgaatgttagttcgaaaagcaaaaaggccatcctttcggatggcctttcgcttgatttgatgtctggcagtttatggcgggcgtcctgcccgccaccctccgggccgttgcttcgcaacgttcaaatccgctcccggcggatttgtcctactcgggagagtgttcaccgacaaacaacagataaaacaaaaggcccagtcttccgactgagccttttgttttatttgatgtctggcagttccctactctcgcatggggagaccccacactaccatcggcgctacggcggtttcacttctgagttcggcatggggtcaggtgggaccaccgcgctactgccgccagacaaattcttttctaatctgccgaactttaacctaaaaagtggtgctgatacccagagtcgaactggggacctcacccttaccaagggtgcgctctaccaactgagccatatcagcacgctaaatttgatgcctggcagttccctactctcgcatggggagaccccacactaccatcggcgctacggcgtttcacttctgagttcggcatggggtcaggtgggaccaccgcgctacggccgccaggcaaattctgttttatcagaccgcttctgcgttctgatttaatctgtatcaggctgaaaatcttctctcatccggataacaatttcacacaggaaacagctatgaccatgattacgccaagctcgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 19 - DNA, 1053 bp, Escherichia coli
  gene (1)..(1053): DNA sequence of the coding region of the wild type aroG gene
atgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaa

SEQ ID NO: 20 - DNA, 8820 bp, Escherichia coli
  DNA sequence of the plasmid pMH28F (1)..(8820)
  misc_feature (5894)..(5894): n is a, c, g, or t
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagttacctagagagggtgagaattgccgaacatgcgcataagtttcccggacagatttcaggtggtcagcagcaacgcgttgccattgcgcgttcgctgtgtatgaagccgaaaattatgttgtttgatgagccaacgtcggcgctcgatcctgagatggtgaaagaggtgctggatacgatgattgggctggcgcagtcgggtatgacaatgttgtgtgtaacacatgagatggggtttgcacgaaccgtcgctgaccgggtaatttttatggatcgtggggaaatagtggagcaagctgcacctgatgaattttttgcgcatcctaaatcagagcgtacgagggcatttttatcgcaggtaatccattaattgaatgttagttcgaaaagcaaaaaggccatcctttcggatggcctttcgcttgatttgatgtctggcagtttatggcgggcgtcctgcccgccaccctccgggccgttgcttcgcaacgttcaaatccggtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaatttttttaaggcagttattggtgcccttaaacgcctggtgctacgcctgaataagtgataataagcggatgaatggcagaaattcgaaagcaaattcgacccggtcgtcggttcagggcagggtcgttaaatagccgcttatgtctattgctggtttantcggtacccggggatcgcggccgcggaccggatcccatcacatatacctgccgttcactattatttagtgaaatgagatattatgatattttctgaattgtgattaaaaaggcaactttatgcccatgcaacagaaactataaaaaatacagagaatgaaaagaaacagatagattttttagttctttaggcccgtagtctgcaaatccttttatgattttctatcaaacaaaagaggaaaatagaccagttgcaatccaaacgagagtctaatagaatgaggtcgaaaagtaaatcgcgcgggtttgttactgataaagcaggcaagacctaaaatgtgtaaagggcaaagtgtatactttggcgtcaccccttacatattttaggtctttttttattgtgcgtaactaacttgccatcttcaaacaggagggctggaagaagcagaccgctaacacagtacataaaaaaggagacatgaacgatgaacatcaaaaagtttgcaaaacaagcaacagtattaacctttactaccgcactgctggcaggaggcgcaactcaagcgtttgcgaaagaaacgaaccaaaagccatataaggaaacatacggcatttcccatattacacgccatgatatgctgcaaatccctgaacagcaaaaaaatgaaaaatatcaagttcctgaattcgattcgtccacaattaaaaatatctcttctgcaaaaggcctggacgtttgggacagctggccattacaaaacgctgacggcactgtcgcaaactatcacggctaccacatcgtctttgcattagccggagatcctaaaaatgcggatgacacatcgatttacatgttctatcaaaaagtcggcgaaacttctattgacagctggaaaaacgctggccgcgtctttaaagacagcgacaaattcgatgcaaatgattctatcctaaaagaccaaacacaagaatggtcaggttcagccacatttacatctgacggaaaaatccgtttattctacactgatttctccggtaaacattacggcaaacaaacactgacaactgcacaagttaacgtatcagcatcagacagctctttgaacatcaacggtgtagaggattataaatcaatctttgacggtgacggaaaaacgtatcaaaatgtacagcagttcatcgatgaaggcaactacagctcaggcgacaaccatacgctgagagatcctcactacgtagaagataaaggccacaaatacttagtatttgaagcaaacactggaactgaagatggctaccaaggcgaagaatctttatttaacaaagcatactatggcaaaagcacatcattcttccgtcaagaaagtcaaaaacttctgcaaagcgataaaaaacgcacggctgagttagcaaacggcgctctcggtatgattgagctaaacgatgattacacactgaaaaaagtgatgaaaccgctgattgcatctaacacagtaacagatgaaattgaacgcgcgaacgtctttaaaatgaacggcaaatggtacctgttcactgactcccgcggatcaaaaatgacgattgacggcattacgtctaacgatatttacatgcttggttatgtttctaattctttaactggcccatacaagccgctgaacaaaactggccttgtgttaaaaatggatcttgatcctaacgatgtaacctttacttactcacacttcgctgtacctcaagcgaaaggaaacaatgtcgtgattacaagctatatgacaaacagaggattctacgcagacaaacaatcaacgtttgcgccgagcttcctgctgaacatcaaaggcaagaaaacatctgttgtcaaagacagcatccttgaacaaggacaattaacagttaacaaataaaaacgcaaaagaaaatgccaatatcctattggcattttcttttatttcttccatttaaatggatgcatgcgctagcggagtgtatactggcttactatgttggcactgatgagggtgtcagtgaagtgcttcctcccggcggatttgtcctactcgggagagtgttcaccgacaaacaacagataaaacaaaaggcccagtcttccgactgagccttttgttttatttgatgtctggcagttccctactctcgcatggggagaccccacactaccatcggcgctacggcggtttcacttctgagttcggcatggggtcaggtgggaccaccgcgctactgccgccagacaaattcttttctaatctgccgaactttaacctaaaaagtggtgctgatacccagagtcgaactggggacctcacccttaccaagggtgcgctctaccaactgagccatatcagcacgctaaatttgatgcctggcagttccctactctcgcatggggagaccccacactaccatcggcgctacggcgtttcacttctgagttcggcatggggtcaggtgggaccaccgcgctacggccgccaggcaaattctgttttatcagaccgcttctgcgttctgatttaatctgtatcaggctgaaaatcttctctcatccggataacaatttcacacaggaaacagctatgaccatgattacgccaagctcgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 21 - DNA, 4774 bp, Escherichia coli
  DNA sequence of the plasmid pCL1921 (1)..(4774)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 22 - DNA, 6432 bp, Escherichia coli
  DNA sequence of the plasmid  pMG27 (1)..(6432)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgaccgttaaatctatcaccgcaagggataaatatctaacaccgtgcgtgttgactattttacctctggcggtgataatggttgcatgtactaatctagataaggaatatagccatgaccgcaccgattcaggatctgcgtgatgcaattgccctgctgcaacagcatgataatcagtatctggaaaccgatcatccggttgatccgaatgcagaactggcaggcgtttatcgtcatattggtgccggtggcaccgttaaacgtccgacccgtattggtccggcaatgatgtttaataacattaaaggttatccgcacagccgtattctggttggtatgcatgcaagccgtcagcgtgcagcactgctgctgggttgtgaagcaagtcagctggcactggaagttggtaaagcagttaaaaaaccggttgcaccggtggttgttccggcaagcagcgcaccgtgtcaagagcagatttttctggcagatgatccggattttgatctgcgtaccctgctgcctgcacataccaataccccgattgatgcaggtccgtttttttgtctgggtctggccctggcaagcgatccggtggatgcaagcctgaccgatgttaccattcatcgtctgtgtgttcagggtcgtgatgaactgagcatgttcctggcagcaggtcgccatattgaagtttttcgtcagaaagcagaagcagcaggtaaaccgctgccgattaccattaatatgggtctggacccagcaatctatattggcgcatgttttgaagcaccgaccaccccgtttggttataatgaactgggtgttgccggtgcactgcgtcagcgtccggttgaactggttcagggtgttagcgttccggaaaaagcaattgcacgtgccgaaattgttattgaaggtgaactgctgcctggtgttcgtgttcgtgaagatcagcataccaattcaggtcatgcaatgccggaatttccgggttattgtggtggtgcaaatccgagcctgccggttattaaagttaaagccgttaccatgcgcaataacgcaattctgcaaaccctggttggtccgggtgaagaacataccaccctggcaggtctgccgaccgaagcaagcatttggaatgcagttgaagcagcaattccgggttttctgcaaaatgtttatgcccataccgcaggcggtggtaaatttctgggtattctgcaagtgaaaaaacgtcagcctgccgatgaaggtcgtcagggtcaggcagccctgctggcgctggcaacctatagcgaactgaaaaatatcattctggtggatgaggatgtggacatttttgatagtgatgatattctgtgggcaatgaccacccgtatgcagggtgatgttagcattaccaccattccgggtattcgcggtcatcagctggacccgagccagacaccggaatattcaccgagcattcgtggtaatggtattagctgcaaaaccatctttgattgtaccgttccgtgggcactgaaaagccattttgaacgtgcaccgtttgcagatgttgatccgcgtccgtttgcacctgaatattttgcacgtctggaaaaaaatcagggcagcgcaaaataagctaataacaggcctgctggtaatcgcaggaatttttatttggatggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 23 - DNA, 7294 bp, Escherichia coli
  DNA sequence of the plasmid  pMG31 (1)..(7294)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctgcagaggaggaatatagccatggaagtgaaaatcttcaacacccaggatgttcaggattttctgcgtgttgcaagcggtctggaacaagagggtggtaatccgcgtgttaaacaaattattcatcgtgttctgagcgacctgtataaagcaattgaagatctgaatatcaccagcgacgaatattgggcaggcgttgcatatctgaatcagctgggtgcaaatcaagaagcaggtctgctgagtccgggtctgggttttgatcattatctggatatgcgtatggatgcagaagatgcagcactgggtattgaaaatgcaacaccgcgtaccattgaaggtccgctgtatgttgcgggtgcaccggaaagcgttggttatgcacgcatggatgatggtagcgatccgaatggtcataccctgattctgcatggcaccatttttgatgcagatggtaaaccgctgccgaatgcaaaagttgaaatttggcatgcaaacaccaaaggcttttatagccattttgatccgaccggtgaacagcaggcctttaatatgcgtcgtagcattattaccgatgagaatggtcagtatcgtgttcgtaccattctgcctgccggttatggttgtcctccggaaggtccgacccagcaactgctgaaccaactgggtcgtcatggtaatcgtccggcacatattcattattttgttagcgcagatggtcaccgtaaactgaccacccagattaatgttgccggtgatccgtatacctatgatgattttgcatatgccacccgtgaaggtctggttgttgatgcagttgaacataccgatccggaagcaattaaagccaatgatgtggaaggtccttttgccgaaatggtgtttgatctgaaactgacccgtctggttgatggtgttgataatcaggttgtggatcgtccgcgtctggcagtttaatacaccaaaatggttcaaaattatcaggcgagtgatcatgatcactggcctgtttttatttcagggaagggtggagacaattacgtggataatcagatcatccaagaaaccgtggataaaattctgagcgttctgccgaatcaggcaggtcagctggcacgtctggtgcgtctgatgcaatttgcatgcgatccgaccattaccgttattggcaaatataaccatggtaaaagccgtctgctgaatgaactgattggcaccgatatctttagcgttgcagataaacgtgaaaccattcagctggccgaacataaacaggatcaggttcgttggctggatgcacctggtctggatgccgatgttgcagcagttgatgatcgtcatgcatttgaagcagtttggacccaggcagatattcgtctgtttgttcatagcgttcgtgaaggtgaactggatgcaaccgaacaccatctgctgcaacagctgattgaagatgccgatcatagccgtcgtcagaccattctggttctgacccagattgatcagattccggatcagaccatcctgacacagattaaaaccagcattgcacagcaggttccgaaactggatatttgggcagttagcgcaacccgtcatcgtcagggcattgaaaacggtaaaaccctgctgatcgaaaaaagcggtattggtgcactgcgccataccctggaacaggcactggcacaggtgccgagcgcacgtacctatgaaaaaaatcgtctgctgtcagatctgcaccatcagctgaaacaactgctgctggatcagaaacatgttctgcaacaactgcaacagacacagcaacagcagctgcatgattttgataccggtctgattaacattctggacaaaattcgtgttgatctggaaccgattgtgaatattgatggtcaggatcaagcactgaatccggatagctttgcaaccatgtttaaaaacaccgcagcaaaacagcagcgtgccaaagttcagattgcatatagccgtgcatgcattgaaatcaacagccatctgattcgccatggtgttgttggtctgcctgcggaacagcagaccaccattaaaagcattgataccgtgattgttgccgtgtttggtatcagcgttaaatttcgtgatcagctgcgtgccctgttttataccgataccgaacgtcagcgtctgcaacgtgaatttcgtttctattttgaaaaaagtgccggtcgcatgattctggcagcaaaaattgaacagaccatgcgtcagcagggctgtattcagaatgccatgatggcactgcaacaaatggaaagcgcagcataaaaacacggacgccgcaaacggcgtccgaatttcttggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 24 - DNA, 8952 bp, Escherichia coli
  DNA sequence of the plasmid  pMG33 (1)..(8952)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctgcagaggaggaatatagccatggaagtgaaaatcttcaacacccaggatgttcaggattttctgcgtgttgcaagcggtctggaacaagagggtggtaatccgcgtgttaaacaaattattcatcgtgttctgagcgacctgtataaagcaattgaagatctgaatatcaccagcgacgaatattgggcaggcgttgcatatctgaatcagctgggtgcaaatcaagaagcaggtctgctgagtccgggtctgggttttgatcattatctggatatgcgtatggatgcagaagatgcagcactgggtattgaaaatgcaacaccgcgtaccattgaaggtccgctgtatgttgcgggtgcaccggaaagcgttggttatgcacgcatggatgatggtagcgatccgaatggtcataccctgattctgcatggcaccatttttgatgcagatggtaaaccgctgccgaatgcaaaagttgaaatttggcatgcaaacaccaaaggcttttatagccattttgatccgaccggtgaacagcaggcctttaatatgcgtcgtagcattattaccgatgagaatggtcagtatcgtgttcgtaccattctgcctgccggttatggttgtcctccggaaggtccgacccagcaactgctgaaccaactgggtcgtcatggtaatcgtccggcacatattcattattttgttagcgcagatggtcaccgtaaactgaccacccagattaatgttgccggtgatccgtatacctatgatgattttgcatatgccacccgtgaaggtctggttgttgatgcagttgaacataccgatccggaagcaattaaagccaatgatgtggaaggtccttttgccgaaatggtgtttgatctgaaactgacccgtctggttgatggtgttgataatcaggttgtggatcgtccgcgtctggcagtttaatacaccaaaatggttcaaaattatcaggcgagtgatcatgatcactggcctgtttttatttcagggaagggtggagacaattacgtggataatcagatcatccaagaaaccgtggataaaattctgagcgttctgccgaatcaggcaggtcagctggcacgtctggtgcgtctgatgcaatttgcatgcgatccgaccattaccgttattggcaaatataaccatggtaaaagccgtctgctgaatgaactgattggcaccgatatctttagcgttgcagataaacgtgaaaccattcagctggccgaacataaacaggatcaggttcgttggctggatgcacctggtctggatgccgatgttgcagcagttgatgatcgtcatgcatttgaagcagtttggacccaggcagatattcgtctgtttgttcatagcgttcgtgaaggtgaactggatgcaaccgaacaccatctgctgcaacagctgattgaagatgccgatcatagccgtcgtcagaccattctggttctgacccagattgatcagattccggatcagaccatcctgacacagattaaaaccagcattgcacagcaggttccgaaactggatatttgggcagttagcgcaacccgtcatcgtcagggcattgaaaacggtaaaaccctgctgatcgaaaaaagcggtattggtgcactgcgccataccctggaacaggcactggcacaggtgccgagcgcacgtacctatgaaaaaaatcgtctgctgtcagatctgcaccatcagctgaaacaactgctgctggatcagaaacatgttctgcaacaactgcaacagacacagcaacagcagctgcatgattttgataccggtctgattaacattctggacaaaattcgtgttgatctggaaccgattgtgaatattgatggtcaggatcaagcactgaatccggatagctttgcaaccatgtttaaaaacaccgcagcaaaacagcagcgtgccaaagttcagattgcatatagccgtgcatgcattgaaatcaacagccatctgattcgccatggtgttgttggtctgcctgcggaacagcagaccaccattaaaagcattgataccgtgattgttgccgtgtttggtatcagcgttaaatttcgtgatcagctgcgtgccctgttttataccgataccgaacgtcagcgtctgcaacgtgaatttcgtttctattttgaaaaaagtgccggtcgcatgattctggcagcaaaaattgaacagaccatgcgtcagcagggctgtattcagaatgccatgatggcactgcaacaaatggaaagcgcagcataaaaacacggacgccgcaaacggcgtccgaatttcttggtcgaccgttaaatctatcaccgcaagggataaatatctaacaccgtgcgtgttgactattttacctctggcggtgataatggttgcatgtactaatctagataaggaatatagccatgaccgcaccgattcaggatctgcgtgatgcaattgccctgctgcaacagcatgataatcagtatctggaaaccgatcatccggttgatccgaatgcagaactggcaggcgtttatcgtcatattggtgccggtggcaccgttaaacgtccgacccgtattggtccggcaatgatgtttaataacattaaaggttatccgcacagccgtattctggttggtatgcatgcaagccgtcagcgtgcagcactgctgctgggttgtgaagcaagtcagctggcactggaagttggtaaagcagttaaaaaaccggttgcaccggtggttgttccggcaagcagcgcaccgtgtcaagagcagatttttctggcagatgatccggattttgatctgcgtaccctgctgcctgcacataccaataccccgattgatgcaggtccgtttttttgtctgggtctggccctggcaagcgatccggtggatgcaagcctgaccgatgttaccattcatcgtctgtgtgttcagggtcgtgatgaactgagcatgttcctggcagcaggtcgccatattgaagtttttcgtcagaaagcagaagcagcaggtaaaccgctgccgattaccattaatatgggtctggacccagcaatctatattggcgcatgttttgaagcaccgaccaccccgtttggttataatgaactgggtgttgccggtgcactgcgtcagcgtccggttgaactggttcagggtgttagcgttccggaaaaagcaattgcacgtgccgaaattgttattgaaggtgaactgctgcctggtgttcgtgttcgtgaagatcagcataccaattcaggtcatgcaatgccggaatttccgggttattgtggtggtgcaaatccgagcctgccggttattaaagttaaagccgttaccatgcgcaataacgcaattctgcaaaccctggttggtccgggtgaagaacataccaccctggcaggtctgccgaccgaagcaagcatttggaatgcagttgaagcagcaattccgggttttctgcaaaatgtttatgcccataccgcaggcggtggtaaatttctgggtattctgcaagtgaaaaaacgtcagcctgccgatgaaggtcgtcagggtcaggcagccctgctggcgctggcaacctatagcgaactgaaaaatatcattctggtggatgaggatgtggacatttttgatagtgatgatattctgtgggcaatgaccacccgtatgcagggtgatgttagcattaccaccattccgggtattcgcggtcatcagctggacccgagccagacaccggaatattcaccgagcattcgtggtaatggtattagctgcaaaaccatctttgattgtaccgttccgtgggcactgaaaagccattttgaacgtgcaccgtttgcagatgttgatccgcgtccgtttgcacctgaatattttgcacgtctggaaaaaaatcagggcagcgcaaaataagctaataacaggcctgctggtaatcgcaggaatttttatttggatggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 25 - DNA, 10630 bp, Escherichia coli
  DNA sequence of the plasmid  pMG37 (1)..(10630)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctgcagaggaggaatatagccatggaagtgaaaatcttcaacacccaggatgttcaggattttctgcgtgttgcaagcggtctggaacaagagggtggtaatccgcgtgttaaacaaattattcatcgtgttctgagcgacctgtataaagcaattgaagatctgaatatcaccagcgacgaatattgggcaggcgttgcatatctgaatcagctgggtgcaaatcaagaagcaggtctgctgagtccgggtctgggttttgatcattatctggatatgcgtatggatgcagaagatgcagcactgggtattgaaaatgcaacaccgcgtaccattgaaggtccgctgtatgttgcgggtgcaccggaaagcgttggttatgcacgcatggatgatggtagcgatccgaatggtcataccctgattctgcatggcaccatttttgatgcagatggtaaaccgctgccgaatgcaaaagttgaaatttggcatgcaaacaccaaaggcttttatagccattttgatccgaccggtgaacagcaggcctttaatatgcgtcgtagcattattaccgatgagaatggtcagtatcgtgttcgtaccattctgcctgccggttatggttgtcctccggaaggtccgacccagcaactgctgaaccaactgggtcgtcatggtaatcgtccggcacatattcattattttgttagcgcagatggtcaccgtaaactgaccacccagattaatgttgccggtgatccgtatacctatgatgattttgcatatgccacccgtgaaggtctggttgttgatgcagttgaacataccgatccggaagcaattaaagccaatgatgtggaaggtccttttgccgaaatggtgtttgatctgaaactgacccgtctggttgatggtgttgataatcaggttgtggatcgtccgcgtctggcagtttaatacaccaaaatggttcaaaattatcaggcgagtgatcatgatcactggcctgtttttatttcagggaagggtggagacaattacgtggataatcagatcatccaagaaaccgtggataaaattctgagcgttctgccgaatcaggcaggtcagctggcacgtctggtgcgtctgatgcaatttgcatgcgatccgaccattaccgttattggcaaatataaccatggtaaaagccgtctgctgaatgaactgattggcaccgatatctttagcgttgcagataaacgtgaaaccattcagctggccgaacataaacaggatcaggttcgttggctggatgcacctggtctggatgccgatgttgcagcagttgatgatcgtcatgcatttgaagcagtttggacccaggcagatattcgtctgtttgttcatagcgttcgtgaaggtgaactggatgcaaccgaacaccatctgctgcaacagctgattgaagatgccgatcatagccgtcgtcagaccattctggttctgacccagattgatcagattccggatcagaccatcctgacacagattaaaaccagcattgcacagcaggttccgaaactggatatttgggcagttagcgcaacccgtcatcgtcagggcattgaaaacggtaaaaccctgctgatcgaaaaaagcggtattggtgcactgcgccataccctggaacaggcactggcacaggtgccgagcgcacgtacctatgaaaaaaatcgtctgctgtcagatctgcaccatcagctgaaacaactgctgctggatcagaaacatgttctgcaacaactgcaacagacacagcaacagcagctgcatgattttgataccggtctgattaacattctggacaaaattcgtgttgatctggaaccgattgtgaatattgatggtcaggatcaagcactgaatccggatagctttgcaaccatgtttaaaaacaccgcagcaaaacagcagcgtgccaaagttcagattgcatatagccgtgcatgcattgaaatcaacagccatctgattcgccatggtgttgttggtctgcctgcggaacagcagaccaccattaaaagcattgataccgtgattgttgccgtgtttggtatcagcgttaaatttcgtgatcagctgcgtgccctgttttataccgataccgaacgtcagcgtctgcaacgtgaatttcgtttctattttgaaaaaagtgccggtcgcatgattctggcagcaaaaattgaacagaccatgcgtcagcagggctgtattcagaatgccatgatggcactgcaacaaatggaaagcgcagcataaaaacacggacgccgcaaacggcgtccgaatttcttggtcgaccgttaaatctatcaccgcaagggataaatatctaacaccgtgcgtgttgactattttacctctggcggtgataatggttgcatgtactaatctagataaggaatatagccatgaccgcaccgattcaggatctgcgtgatgcaattgccctgctgcaacagcatgataatcagtatctggaaaccgatcatccggttgatccgaatgcagaactggcaggcgtttatcgtcatattggtgccggtggcaccgttaaacgtccgacccgtattggtccggcaatgatgtttaataacattaaaggttatccgcacagccgtattctggttggtatgcatgcaagccgtcagcgtgcagcactgctgctgggttgtgaagcaagtcagctggcactggaagttggtaaagcagttaaaaaaccggttgcaccggtggttgttccggcaagcagcgcaccgtgtcaagagcagatttttctggcagatgatccggattttgatctgcgtaccctgctgcctgcacataccaataccccgattgatgcaggtccgtttttttgtctgggtctggccctggcaagcgatccggtggatgcaagcctgaccgatgttaccattcatcgtctgtgtgttcagggtcgtgatgaactgagcatgttcctggcagcaggtcgccatattgaagtttttcgtcagaaagcagaagcagcaggtaaaccgctgccgattaccattaatatgggtctggacccagcaatctatattggcgcatgttttgaagcaccgaccaccccgtttggttataatgaactgggtgttgccggtgcactgcgtcagcgtccggttgaactggttcagggtgttagcgttccggaaaaagcaattgcacgtgccgaaattgttattgaaggtgaactgctgcctggtgttcgtgttcgtgaagatcagcataccaattcaggtcatgcaatgccggaatttccgggttattgtggtggtgcaaatccgagcctgccggttattaaagttaaagccgttaccatgcgcaataacgcaattctgcaaaccctggttggtccgggtgaagaacataccaccctggcaggtctgccgaccgaagcaagcatttggaatgcagttgaagcagcaattccgggttttctgcaaaatgtttatgcccataccgcaggcggtggtaaatttctgggtattctgcaagtgaaaaaacgtcagcctgccgatgaaggtcgtcagggtcaggcagccctgctggcgctggcaacctatagcgaactgaaaaatatcattctggtggatgaggatgtggacatttttgatagtgatgatattctgtgggcaatgaccacccgtatgcagggtgatgttagcattaccaccattccgggtattcgcggtcatcagctggacccgagccagacaccggaatattcaccgagcattcgtggtaatggtattagctgcaaaaccatctttgattgtaccgttccgtgggcactgaaaagccattttgaacgtgcaccgtttgcagatgttgatccgcgtccgtttgcacctgaatattttgcacgtctggaaaaaaatcagggcagcgcaaaataagctaataacaggcctgctggtaatcgcaggaatttttatttggatggatccgcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctcccgggttgaggaaaacctaatgaaactgaccagcctgcgtgttagcctgctggcactgggtctggttaccagcggttttgcagcagcagaaacctataccgttgatcgttatcaggatgatagcgaaaaaggtagcctgcgttgggcaattgaacagagcaatgcaaatagcgcacaagaaaaccagattctgattcaggcagttggtaaagcaccgtatgttatcaaagttgataaaccgctgcctccgattaaaagcagcgttaaaatcattggcaccgagtgggataaaaccggtgaatttattgcaattgatggcagcaactatatcaaaggcgaaggtgaaaaagcatgtccgggtgcaaatccgggtcagtatggcaccaatgttcgtaccatgaccctgcctggtctggttctgcaagatgttaatggtgttaccctgaaaggtctggatgttcatcgtttttgtattggtgttctggttaatcgcagcagcaataacctgattcagcataatcgtatcagcaacaattatggtggtgccggtgttatgattaccggtgatgatggtaaaggtaatccgaccagcaccaccaccaataataacaaagttctggataacgtgttcatcgataatggtgatggtctggaactgacccgtggtgcagcatttaatctgattgcaaataacctgtttaccagcacaaaagccaatccggaaccgagccagggtattgaaattctgtggggtaatgataatgccgtggtgggtaacaaattcgaaaactattcagatggcctgcaaatcaattggggtaaacgtaactatatcgcctataacgaactgaccaataacagcctgggtttcaatctgacaggtgatggtaacattttcgacagcaataaagtgcatggtaaccgtattggtattgccattcgtagtgaaaaagatgccaatgcacgtattaccctgaccaaaaatcagatttgggataacggcaaagatatcaaacgttgtgaagccggtggtagctgtgttccgaatcagcgtctgggtgcaattgtttttggtgttccggcactggaacatgaaggttttgttggtagccgtggcggtggtgttgttattgaaccggcaaaactgcaaaaaacctgcacccagccgaaccagcagaattgtaatgcaattcctaatcagggtattcaggcaccgaaactgacagttagcaaaaaacagctgaccgttgaagttaaaggcacccctaatcagcgttataatgtggaattttttggcaatcgtaatgccagcagcagcgaagcagaacagtatctgggtagcattgttgttgttaccgatcatcagggtctggcaaaagcaaattgggctccgaaagttagcatgccgagcgttaccgcaaatgtgacagatcatctgggtgcgaccagcgaactgagcagcgcagttaaaatgcgttaaatgcatgcgcgccgcgttcgcgcggcgcttttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 26 - DNA, 6452 bp, Escherichai coli
  DNA sequence of the plasmid  pMG39 (1)..(6452)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccgcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctcccgggttgaggaaaacctaatgaaactgaccagcctgcgtgttagcctgctggcactgggtctggttaccagcggttttgcagcagcagaaacctataccgttgatcgttatcaggatgatagcgaaaaaggtagcctgcgttgggcaattgaacagagcaatgcaaatagcgcacaagaaaaccagattctgattcaggcagttggtaaagcaccgtatgttatcaaagttgataaaccgctgcctccgattaaaagcagcgttaaaatcattggcaccgagtgggataaaaccggtgaatttattgcaattgatggcagcaactatatcaaaggcgaaggtgaaaaagcatgtccgggtgcaaatccgggtcagtatggcaccaatgttcgtaccatgaccctgcctggtctggttctgcaagatgttaatggtgttaccctgaaaggtctggatgttcatcgtttttgtattggtgttctggttaatcgcagcagcaataacctgattcagcataatcgtatcagcaacaattatggtggtgccggtgttatgattaccggtgatgatggtaaaggtaatccgaccagcaccaccaccaataataacaaagttctggataacgtgttcatcgataatggtgatggtctggaactgacccgtggtgcagcatttaatctgattgcaaataacctgtttaccagcacaaaagccaatccggaaccgagccagggtattgaaattctgtggggtaatgataatgccgtggtgggtaacaaattcgaaaactattcagatggcctgcaaatcaattggggtaaacgtaactatatcgcctataacgaactgaccaataacagcctgggtttcaatctgacaggtgatggtaacattttcgacagcaataaagtgcatggtaaccgtattggtattgccattcgtagtgaaaaagatgccaatgcacgtattaccctgaccaaaaatcagatttgggataacggcaaagatatcaaacgttgtgaagccggtggtagctgtgttccgaatcagcgtctgggtgcaattgtttttggtgttccggcactggaacatgaaggttttgttggtagccgtggcggtggtgttgttattgaaccggcaaaactgcaaaaaacctgcacccagccgaaccagcagaattgtaatgcaattcctaatcagggtattcaggcaccgaaactgacagttagcaaaaaacagctgaccgttgaagttaaaggcacccctaatcagcgttataatgtggaattttttggcaatcgtaatgccagcagcagcgaagcagaacagtatctgggtagcattgttgttgttaccgatcatcagggtctggcaaaagcaaattgggctccgaaagttagcatgccgagcgttaccgcaaatgtgacagatcatctgggtgcgaccagcgaactgagcagcgcagttaaaatgcgttaaatgcatgcgcgccgcgttcgcgcggcgcttttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 27 - DNA, 10012 bp, Escherichia coli
  DNA sequence of the plasmid  pMG47 (1)..(10012)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctgcagaggaggaatatagccatggaagtgaaaatcttcaacacccaggatgttcaggattttctgcgtgttgcaagcggtctggaacaagagggtggtaatccgcgtgttaaacaaattattcatcgtgttctgagcgacctgtataaagcaattgaagatctgaatatcaccagcgacgaatattgggcaggcgttgcatatctgaatcagctgggtgcaaatcaagaagcaggtctgctgagtccgggtctgggttttgatcattatctggatatgcgtatggatgcagaagatgcagcactgggtattgaaaatgcaacaccgcgtaccattgaaggtccgctgtatgttgcgggtgcaccggaaagcgttggttatgcacgcatggatgatggtagcgatccgaatggtcataccctgattctgcatggcaccatttttgatgcagatggtaaaccgctgccgaatgcaaaagttgaaatttggcatgcaaacaccaaaggcttttatagccattttgatccgaccggtgaacagcaggcctttaatatgcgtcgtagcattattaccgatgagaatggtcagtatcgtgttcgtaccattctgcctgccggttatggttgtcctccggaaggtccgacccagcaactgctgaaccaactgggtcgtcatggtaatcgtccggcacatattcattattttgttagcgcagatggtcaccgtaaactgaccacccagattaatgttgccggtgatccgtatacctatgatgattttgcatatgccacccgtgaaggtctggttgttgatgcagttgaacataccgatccggaagcaattaaagccaatgatgtggaaggtccttttgccgaaatggtgtttgatctgaaactgacccgtctggttgatggtgttgataatcaggttgtggatcgtccgcgtctggcagtttaatacaccaaaatggttcaaaattatcaggcgagtgatcatgatcactggcctgtttttatttcagggaagggtggagacaattacgtggataatcagatcatccaagaaaccgtggataaaattctgagcgttctgccgaatcaggcaggtcagctggcacgtctggtgcgtctgatgcaatttgcatgcgatccgaccattaccgttattggcaaatataaccatggtaaaagccgtctgctgaatgaactgattggcaccgatatctttagcgttgcagataaacgtgaaaccattcagctggccgaacataaacaggatcaggttcgttggctggatgcacctggtctggatgccgatgttgcagcagttgatgatcgtcatgcatttgaagcagtttggacccaggcagatattcgtctgtttgttcatagcgttcgtgaaggtgaactggatgcaaccgaacaccatctgctgcaacagctgattgaagatgccgatcatagccgtcgtcagaccattctggttctgacccagattgatcagattccggatcagaccatcctgacacagattaaaaccagcattgcacagcaggttccgaaactggatatttgggcagttagcgcaacccgtcatcgtcagggcattgaaaacggtaaaaccctgctgatcgaaaaaagcggtattggtgcactgcgccataccctggaacaggcactggcacaggtgccgagcgcacgtacctatgaaaaaaatcgtctgctgtcagatctgcaccatcagctgaaacaactgctgctggatcagaaacatgttctgcaacaactgcaacagacacagcaacagcagctgcatgattttgataccggtctgattaacattctggacaaaattcgtgttgatctggaaccgattgtgaatattgatggtcaggatcaagcactgaatccggatagctttgcaaccatgtttaaaaacaccgcagcaaaacagcagcgtgccaaagttcagattgcatatagccgtgcatgcattgaaatcaacagccatctgattcgccatggtgttgttggtctgcctgcggaacagcagaccaccattaaaagcattgataccgtgattgttgccgtgtttggtatcagcgttaaatttcgtgatcagctgcgtgccctgttttataccgataccgaacgtcagcgtctgcaacgtgaatttcgtttctattttgaaaaaagtgccggtcgcatgattctggcagcaaaaattgaacagaccatgcgtcagcagggctgtattcagaatgccatgatggcactgcaacaaatggaaagcgcagcataaaaacacggacgccgcaaacggcgtccgaatttcttggtcgaccgttaaatctatcaccgcaagggataaatatctaacaccgtgcgtgttgactattttacctctggcggtgataatggttgcatgtactaatctagataaggaatatagccatgaccgcaccgattcaggatctgcgtgatgcaattgccctgctgcaacagcatgataatcagtatctggaaaccgatcatccggttgatccgaatgcagaactggcaggcgtttatcgtcatattggtgccggtggcaccgttaaacgtccgacccgtattggtccggcaatgatgtttaataacattaaaggttatccgcacagccgtattctggttggtatgcatgcaagccgtcagcgtgcagcactgctgctgggttgtgaagcaagtcagctggcactggaagttggtaaagcagttaaaaaaccggttgcaccggtggttgttccggcaagcagcgcaccgtgtcaagagcagatttttctggcagatgatccggattttgatctgcgtaccctgctgcctgcacataccaataccccgattgatgcaggtccgtttttttgtctgggtctggccctggcaagcgatccggtggatgcaagcctgaccgatgttaccattcatcgtctgtgtgttcagggtcgtgatgaactgagcatgttcctggcagcaggtcgccatattgaagtttttcgtcagaaagcagaagcagcaggtaaaccgctgccgattaccattaatatgggtctggacccagcaatctatattggcgcatgttttgaagcaccgaccaccccgtttggttataatgaactgggtgttgccggtgcactgcgtcagcgtccggttgaactggttcagggtgttagcgttccggaaaaagcaattgcacgtgccgaaattgttattgaaggtgaactgctgcctggtgttcgtgttcgtgaagatcagcataccaattcaggtcatgcaatgccggaatttccgggttattgtggtggtgcaaatccgagcctgccggttattaaagttaaagccgttaccatgcgcaataacgcaattctgcaaaccctggttggtccgggtgaagaacataccaccctggcaggtctgccgaccgaagcaagcatttggaatgcagttgaagcagcaattccgggttttctgcaaaatgtttatgcccataccgcaggcggtggtaaatttctgggtattctgcaagtgaaaaaacgtcagcctgccgatgaaggtcgtcagggtcaggcagccctgctggcgctggcaacctatagcgaactgaaaaatatcattctggtggatgaggatgtggacatttttgatagtgatgatattctgtgggcaatgaccacccgtatgcagggtgatgttagcattaccaccattccgggtattcgcggtcatcagctggacccgagccagacaccggaatattcaccgagcattcgtggtaatggtattagctgcaaaaccatctttgattgtaccgttccgtgggcactgaaaagccattttgaacgtgcaccgtttgcagatgttgatccgcgtccgtttgcacctgaatattttgcacgtctggaaaaaaatcagggcagcgcaaaataagctaataacaggcctgctggtaatcgcaggaatttttatttggatggatccgcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctcccgggttgaggaaaacctaatgaaatatagcctgtgcaccattagctttcgtcaccagctgattagctttaccgatattgttcagtttgcctatgaaaacggctttgaaggtattgaactgtggggcacccatgcacagaatctgtatatgcaagaatatgaaaccaccgaacgtgaactgaattgcctgaaagataaaaccctggaaattaccatgatcagcgattatctggatattagcctgagcgcagattttgaaaaaaccatcgaaaaatgtgaacagctggcaattctggccaattggtttaaaacgaacaaaattcgtacctttgccggtcagaaaggtagtgcagattttagccagcaagaacgtcaagagtatgtgaatcgtattcgcatgatttgtgaactgtttgcccagcataatatgtatgttctgctggaaacccatccgaataccctgaccgataccctgccgagcaccctggaactgctgggtgaagttgatcatccgaatctgaaaatcaacctggattttctgcatatctgggaaagcggtgcagatccggttgatagctttcagcagctgcgtccgtggattcagcattatcactttaaaaacattagcagcgcagactatctgcatgtgtttgaaccgaataatgtttatgcagcagcaggtaatcgtaccggtatggttccgctgtttgaaggcattgttaactatgatgaaatcatccaagaagtgcgcgataccgatcattttgcaagcctggaatggtttggtcataacgcaaaagatattctgaaagccgaaatgaaagtgctgaccaatcgtaatctggaagttgttaccagctaaatgcatgcgcgccgcgttcgcgcggcgcttttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 28 - DNA, 10249 bp, Escherichia coli
  DNA sequence of the plasmid  pMG70 (1)..(10243)
gttgacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggactagtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacgtaacggactaagaaaaacactacacgatgctttaactgcaaaaattcagctcaccagttttgaggcaaaatttttgagtgacatgcaaagtaagcatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttagatatcaaagggaaaactgtccatatgcacagatgaaaacggtgtaaaaaagatagatacatcagagcttttacgagtttttggtgcatttaaagctgttcaccatgaacagatcgacaatgtaacagatgaacagcatgtaacacctaatagaacaggtgaaaccagtaaaacaaagcaactagaacatgaaattgaacacctgagacaacttgttacagctcaacagtcacacatagacagcctgaaacaggcgatgctgcttatcgaatcaaagctgccgacaacacgggagccagtgacgcctcccgtggggaaaaaatcatggcaattctggaagaaatagcgctttcagccggcaaacctgaagccggatctgcgattctgataacaaactagcaacaccagaacagcccgtttgcgggcagcaaaacccgttatgcttgtaaaccgttttgtgaaaaaatttttaaaataaaaaaggggacctctagggtccccaattaattagtaatataatctattaaaggtcattcaaaaggtcatccaccggatcaattcccctgctcgcgcaggctgggtgccaagctctcgggtaacatcaaggcccgatccttggagcccttgccctcccgcacgatgatcgtgccgtgatcgaaatccagatccttgacccgcagttgcaaaccctcactgatccgcatgcccgttccatacagaagctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatccgcattaaaatctagcgagggctttactaagctgatccggtggatgaccttttgaatgacctttaatagattatattactaattaattggggaccctagaggtccccttttttattttaaaaattttttcacaaaacggtttacaagcatacgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctgcagaggaggaatatagccatggaagtgaaaatcttcaacacccaggatgttcaggattttctgcgtgttgcaagcggtctggaacaagagggtggtaatccgcgtgttaaacaaattattcatcgtgttctgagcgacctgtataaagcaattgaagatctgaatatcaccagcgacgaatattgggcaggcgttgcatatctgaatcagctgggtgcaaatcaagaagcaggtctgctgagtccgggtctgggttttgatcattatctggatatgcgtatggatgcagaagatgcagcactgggtattgaaaatgcaacaccgcgtaccattgaaggtccgctgtatgttgcgggtgcaccggaaagcgttggttatgcacgcatggatgatggtagcgatccgaatggtcataccctgattctgcatggcaccatttttgatgcagatggtaaaccgctgccgaatgcaaaagttgaaatttggcatgcaaacaccaaaggcttttatagccattttgatccgaccggtgaacagcaggcctttaatatgcgtcgtagcattattaccgatgagaatggtcagtatcgtgttcgtaccattctgcctgccggttatggttgtcctccggaaggtccgacccagcaactgctgaaccaactgggtcgtcatggtaatcgtccggcacatattcattattttgttagcgcagatggtcaccgtaaactgaccacccagattaatgttgccggtgatccgtatacctatgatgattttgcatatgccacccgtgaaggtctggttgttgatgcagttgaacataccgatccggaagcaattaaagccaatgatgtggaaggtccttttgccgaaatggtgtttgatctgaaactgacccgtctggttgatggtgttgataatcaggttgtggatcgtccgcgtctggcagtttaatacaccaaaatggttcaaaattatcaggcgagtgatcatgatcactggcctgtttttatttcagggaagggtggagacaattacgtggataatcagatcatccaagaaaccgtggataaaattctgagcgttctgccgaatcaggcaggtcagctggcacgtctggtgcgtctgatgcaatttgcatgcgatccgaccattaccgttattggcaaatataaccatggtaaaagccgtctgctgaatgaactgattggcaccgatatctttagcgttgcagataaacgtgaaaccattcagctggccgaacataaacaggatcaggttcgttggctggatgcacctggtctggatgccgatgttgcagcagttgatgatcgtcatgcatttgaagcagtttggacccaggcagatattcgtctgtttgttcatagcgttcgtgaaggtgaactggatgcaaccgaacaccatctgctgcaacagctgattgaagatgccgatcatagccgtcgtcagaccattctggttctgacccagattgatcagattccggatcagaccatcctgacacagattaaaaccagcattgcacagcaggttccgaaactggatatttgggcagttagcgcaacccgtcatcgtcagggcattgaaaacggtaaaaccctgctgatcgaaaaaagcggtattggtgcactgcgccataccctggaacaggcactggcacaggtgccgagcgcacgtacctatgaaaaaaatcgtctgctgtcagatctgcaccatcagctgaaacaactgctgctggatcagaaacatgttctgcaacaactgcaacagacacagcaacagcagctgcatgattttgataccggtctgattaacattctggacaaaattcgtgttgatctggaaccgattgtgaatattgatggtcaggatcaagcactgaatccggatagctttgcaaccatgtttaaaaacaccgcagcaaaacagcagcgtgccaaagttcagattgcatatagccgtgcatgcattgaaatcaacagccatctgattcgccatggtgttgttggtctgcctgcggaacagcagaccaccattaaaagcattgataccgtgattgttgccgtgtttggtatcagcgttaaatttcgtgatcagctgcgtgccctgttttataccgataccgaacgtcagcgtctgcaacgtgaatttcgtttctattttgaaaaaagtgccggtcgcatgattctggcagcaaaaattgaacagaccatgcgtcagcagggctgtattcagaatgccatgatggcactgcaacaaatggaaagcgcagcataaaaacacggacgccgcaaacggcgtccgaatttcttggtcgaccgttaaatctatcaccgcaagggataaatatctaacaccgtgcgtgttgactattttacctctggcggtgataatggttgcatgtactaatctagataaggaatatagccatgaccgcaccgattcaggatctgcgtgatgcaattgccctgctgcaacagcatgataatcagtatctggaaaccgatcatccggttgatccgaatgcagaactggcaggcgtttatcgtcatattggtgccggtggcaccgttaaacgtccgacccgtattggtccggcaatgatgtttaataacattaaaggttatccgcacagccgtattctggttggtatgcatgcaagccgtcagcgtgcagcactgctgctgggttgtgaagcaagtcagctggcactggaagttggtaaagcagttaaaaaaccggttgcaccggtggttgttccggcaagcagcgcaccgtgtcaagagcagatttttctggcagatgatccggattttgatctgcgtaccctgctgcctgcacataccaataccccgattgatgcaggtccgtttttttgtctgggtctggccctggcaagcgatccggtggatgcaagcctgaccgatgttaccattcatcgtctgtgtgttcagggtcgtgatgaactgagcatgttcctggcagcaggtcgccatattgaagtttttcgtcagaaagcagaagcagcaggtaaaccgctgccgattaccattaatatgggtctggacccagcaatctatattggcgcatgttttgaagcaccgaccaccccgtttggttataatgaactgggtgttgccggtgcactgcgtcagcgtccggttgaactggttcagggtgttagcgttccggaaaaagcaattgcacgtgccgaaattgttattgaaggtgaactgctgcctggtgttcgtgttcgtgaagatcagcataccaattcaggtcatgcaatgccggaatttccgggttattgtggtggtgcaaatccgagcctgccggttattaaagttaaagccgttaccatgcgcaataacgcaattctgcaaaccctggttggtccgggtgaagaacataccaccctggcaggtctgccgaccgaagcaagcatttggaatgcagttgaagcagcaattccgggttttctgcaaaatgtttatgcccataccgcaggcggtggtaaatttctgggtattctgcaagtgaaaaaacgtcagcctgccgatgaaggtcgtcagggtcaggcagccctgctggcgctggcaacctatagcgaactgaaaaatatcattctggtggatgaggatgtggacatttttgatagtgatgatattctgtgggcaatgaccacccgtatgcagggtgatgttagcattaccaccattccgggtattcgcggtcatcagctggacccgagccagacaccggaatattcaccgagcattcgtggtaatggtattagctgcaaaaccatctttgattgtaccgttccgtgggcactgaaaagccattttgaacgtgcaccgtttgcagatgttgatccgcgtccgtttgcacctgaatattttgcacgtctggaaaaaaatcagggcagcgcaaaataagctaataacaggcctgctggtaatcgcaggaatttttatttggatggatccgcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctcccgggttgaggaaaacctaatgccgagcaaactggcaattagcagcatgagcctgggtcgttgttttgcaggtcatagcctggatagtaaactggatgcagcacagcgttatggttatctgggtattgaactgttttatgaggatctggttgatgttgcagaacatctgagcaatgaacgtccgagtccggaaggtccgtttgttgaagcacagattgcagcagcacgtcatattctgcaaatgtgtcaggcacgtggtctggaagttgtttgtctgcaaccgtttatgcattatgatggtctgaatgatcgtgccgaacatgaacgtcgtctggaaaaactggcactgtggattgaactggcacatgaactgcataccgatattattcagattccggcaaattttctgcctgcaaatcaggttagcgataatctggatctgattgttagcgatctgtgtaaagttgcagatattggtgcacaggcactgcctccgattcgttttgcatatgaaagcctgtgttggagcacccgtgttgatctgtgggaacgttgttgggatattgttcagcgtgtggatcgtccgaattttggtatttgtctggatacctttaacatcctgggtcgcatttatgcagatccgaccagcccgagcggtcgtaccccgaatgcaaaagaagcagttcgtaaaagcattgccaatctggttagccgtgtggatgttagcaaagttttttatgttcaggttgtggatgccgaacgtctgagtaaaccgctgctgcctggtcatccgtattataacccggaacagcctgcacgtatgagctggtcacgtaattgtcgtctgttctatggtgaaaccgaatatggtgcatatctgccggttaaagaagttgcacgcgcactgtttcatggtattggttttgaaggttgggttagcctggaactgtttaatcgtcgtatgagcgaagaaggtccggaagttcctgaagaactggccatgcgtggtgcaattagctgggcaaaactggttcaggatctgcgtattccggttgaaggtccgctggttaccatgcctcgtgttagcgcaagcctgtaaatgcatgcgcgccgcgttcgcgcggcgcttttttttggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgaattc

SEQ ID NO: 29 - DNA, 7623 bp, Escherichia coli
  DNA sequence of the plasmid  pCP32AMP (1)..(7623)
acatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactagtatagtctatagtccgtggaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgacttgaaataattaacaaacaaaggagttacagttagaaattgtaggagagatctcgtttttcgcgacaatctggcgtttttcttgctaattccaggattaatccgttcatagtgtaaaaccccgtttacacattctgacggaagatatagattggaagtattgcattcactaagataagtatggcaacactggaacagacatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaaggtttaattgtcggatgcgccgtcagagtggcgtatccgatgaatcaccacaggcctgataagtcgcgcagcgtcgcatcaggcaatgtgctccattgttagcaacaaaaaagccgactcacttgcagtcggctttctcattttaaacgaatgacgtttacttcgctttaccctggtttgcaaccgccgctgctttcgct

SEQ ID NO: 30 - DNA, 7630 bp, Escherichia coli
  DNA sequence of the plasmid  pCP14 (1)..(7630)
ctcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaggaggaggaatcgccatggagaggattgtcgttactctcggggaacgtagttacccaattaccatcgcatctggtttgtttaatgaaccagcttcattcttaccgctgaaatcgggcgagcaggtcatgttggtcaccaacgaaaccctggctcctctgtatctcgataaggtccgcggcgtacttgaacaggcgggtgttaacgtcgatagcgttatcctccctgacggcgagcagtataaaagcctggctgtactcgataccgtctttacggcgttgttacaaaagccgcatggtcgcgatactacgctggtggcgcttggcggcggcgtagtgggcgatctgaccggcttcgcggcggcgagttatcagcgcggtgttcgtttcattcaagtcccgacgacgttactgtcgcaggtcgattcctccgttggcggcaaaactgcggtcaaccatcccctcggtaaaaacatgattggcgcgttctaccagcctgcttcagtggtggtggatctcgactgtctgaaaacgcttcccccgcgtgagttagcgtcggggctggcagaagtcatcaaatacggcattattcttgacggtgcgtttttcaactggctggaagagaatctggatgcgttgttgcgtctggacggtccggcaatggcgtactgtattcgccgttgttgtgaactgaaggcagaagttgtcgccgccgacgagcgcgaaaccgggttacgtgctttactgaatctgggacacacctttggtcatgccattgaagctgaaatggggtatggcaattggttacatggtgaagcggtcgctgcgggtatggtgatggcggcgcggacgtcggaacgtctcgggcagtttagttctgccgaaacgcagcgtattataaccctgctcacgcgggctgggttaccggtcaatgggccgcgcgaaatgtccgcgcaggcgtatttaccgcatatgctgcgtgacaagaaagtccttgcgggagagatgcgcttaattcttccgttggcaattggtaagagtgaagttcgcagcggcgtttcgcacgagcttgttcttaacgccattgccgattgtcaatcagcgtaatcatcgttcatgcctgatgccgctatgtaggccggataaggcgttcacgccgcatccggcaaccgatgcctgatgcgacgcggtcgcgtcttatcaggcctacaggtcgatgccgatatgtacatcgtattcggcaattaatacatagcaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactagtatagtctatagtccgtggaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatga

SEQ ID NO: 31 - DNA, 10015 bp, Escherichia coli
  DNA sequence of the plasmid  pCP50 (1)..(10015)
cttgaaataattaacaaacaaaggagttacagttagaaattgtaggagagatctcgtttttcgcgacaatctggcgtttttcttgctaattccaggattaatccgttcatagtgtaaaaccccgtttacacattctgacggaagatatagattggaagtattgcattcactaagataagtatggcaacactggaacagacatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaaggtttaattgtcggatgcgccgtcagagtggcgtatccgatgaatcaccacaggcctgataagtcgcgcagcgtcgcatcaggcaatgtgctccattgttagcaacaaaaaagccgactcacttgcagtcggctttctcattttaaacgaatgacgtttacttcgctttaccctggtttgcaaccgccgctgctttcgctacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactagtatagtctatagtccgtggaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgaaaggaaaagcgcaacggacgggcgagtagattgcgcaacatgcgagcatgatccagagatttctgaagcagcaaaaggatgttccatgtacatgacgcgcggcttgcggtaaattgttggcaaattttccggcgtagcccaaaacgcgctgtcgtcaagtcgttaagggcgtgcccttcatcatccgatctggagtcaaaatgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggccacccggaagtaggttataccgctggtgtggaaaccaccaccggtccgctgggtcagggtattgccaacgcagtcggtatggcgattgcagaaaaaacgctggcggcgcagtttaaccgtccaggtcacgacattgtcgaccactacacctacgccttcatgggcgacggctgcatgatggaaggcatctcccacgaagtttgctctctggcgggtacgctgaagctgggtaaactgattgcgttctacgatgacaacggtatctcaatcgatggtcacgttgaaggctggttcactgacgacaccgcaatgcgtttcgaagcttacggctggcacgttattcgcgacatcgacggtcatgacgcggcatccatcaaacgcgcagtagaagaagcgcgcgcagtgactgacaaaccgtccctgctgatgtgcaaaaccatcatcggtttcggttccccgaacaaagccggtacccacgactcccacggtgcgccgctgggcgacgctgaaattgccctgacccgcgaacagctgggctggaaatacgcgccgttcgaaatcccgtctgaaatctatgctcagtgggatgcgaaagaagcaggccaggcgaaagaatctgcatggaatgagaagtttgcggcttacgcgaaagcttatccgcaggaagcggctgaatttacccgccgtatgaaaggcgaaatgccgtctgacttcgacgccaaagcgaaagagtttatcgctaaactgcaggctaatccggcgaaaatcgccagccgtaaagcgtcgcagaatgctatcgaagcgttcggcccgctgttgcctgaattcctcggcggctctgctgacctggcaccgtctaacctgaccctgtggtctggttctaaagcaatcaacgaagatgctgcaggtaactacatccactacggtgttcgcgagttcggtatgaccgcgattgctaacggtatctccctgcacggtggtttcctgccgtacacctccaccttcctgatgttcgtggaatacgcacgtaacgccgtacgtatggctgcgctgatgaaacagcgtcaggtgatggtttacacccacgactccatcggtctgggcgaagatggcccgactcaccagccggttgagcaggtcgcttctctgcgcgtgaccccgaacatgtctacatggcgtccgtgtgaccaggttgaatccgcggtcgcgtggaaatacggcgttgagcgtcaggacggcccgactgcgcttatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaattagcatttcgggtaaaaaggtcgcttcggcgaccttttttattaccttgatatgtccgtttgcggacaagcaatagataaagcgtgttgtagatcacaaatatttatatgcaataaatatcaattatgtaatatgcatcacgatatgcgtattgacatttgttgttataactataactcaatgttatataagaaattaa

SEQ ID NO: 32 - DNA, 9065 bp, Escherichia coli
  DNA sequence of the plasmid  pCP54 (1)..(9065)
acatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcagaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactagtatagtctatagtccgtggaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgacttgaaataattaacaaacaaaggagttacagttagaaattgtaggagagatctcgtttttcgcgacaatctggcgtttttcttgctaattccaggattaatccgttcatagtgtaaaaccccgtttacacattctgacggaagatatagattggaagtattgcattcactaagataagtatggcaacactggaacagacatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaaggtttaattgtcggatgcgccgtcagagtggcgtatccgatgaatcaccacaggcctgataagtcgcgcagcgtcgcatcaggcaatgtgctccattgttagcaacaaaaaagccgactcacttgcagtcggctttctcattttaaacgaatgacgtttacttcgctttaccctggtttgcaaccgccgctgctttcgctctcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaggaggaggaatcgccatggagaggattgtcgttactctcggggaacgtagttacccaattaccatcgcatctggtttgtttaatgaaccagcttcattcttaccgctgaaatcgggcgagcaggtcatgttggtcaccaacgaaaccctggctcctctgtatctcgataaggtccgcggcgtacttgaacaggcgggtgttaacgtcgatagcgttatcctccctgacggcgagcagtataaaagcctggctgtactcgataccgtctttacggcgttgttacaaaagccgcatggtcgcgatactacgctggtggcgcttggcggcggcgtagtgggcgatctgaccggcttcgcggcggcgagttatcagcgcggtgttcgtttcattcaagtcccgacgacgttactgtcgcaggtcgattcctccgttggcggcaaaactgcggtcaaccatcccctcggtaaaaacatgattggcgcgttctaccagcctgcttcagtggtggtggatctcgactgtctgaaaacgcttcccccgcgtgagttagcgtcggggctggcagaagtcatcaaatacggcattattcttgacggtgcgtttttcaactggctggaagagaatctggatgcgttgttgcgtctggacggtccggcaatggcgtactgtattcgccgttgttgtgaactgaaggcagaagttgtcgccgccgacgagcgcgaaaccgggttacgtgctttactgaatctgggacacacctttggtcatgccattgaagctgaaatggggtatggcaattggttacatggtgaagcggtcgctgcgggtatggtgatggcggcgcggacgtcggaacgtctcgggcagtttagttctgccgaaacgcagcgtattataaccctgctcacgcgggctgggttaccggtcaatgggccgcgcgaaatgtccgcgcaggcgtatttaccgcatatgctgcgtgacaagaaagtccttgcgggagagatgcgcttaattcttccgttggcaattggtaagagtgaagttcgcagcggcgtttcgcacgagcttgttcttaacgccattgccgattgtcaatcagcgtaatcatcgttcatgcctgatgccgctatgtaggccggataaggcgttcacgccgcatccggcaaccgatgcctgatgcgacgcggtcgcgtcttatcaggcctacaggtcgatgccgatatgtacatcgtattcggcaattaatacatagca

SEQ ID NO: 33 - DNA, 11475 bp, Escherichia coli
  DNA sequence of the plasmid  pCP55 (1)..(11475)
acatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactagtatagtctatagtccgtggaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgaaaggaaaagcgcaacggacgggcgagtagattgcgcaacatgcgagcatgatccagagatttctgaagcagcaaaaggatgttccatgtacatgacgcgcggcttgcggtaaattgttggcaaattttccggcgtagcccaaaacgcgctgtcgtcaagtcgttaagggcgtgcccttcatcatccgatctggagtcaaaatgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggccacccggaagtaggttataccgctggtgtggaaaccaccaccggtccgctgggtcagggtattgccaacgcagtcggtatggcgattgcagaaaaaacgctggcggcgcagtttaaccgtccaggtcacgacattgtcgaccactacacctacgccttcatgggcgacggctgcatgatggaaggcatctcccacgaagtttgctctctggcgggtacgctgaagctgggtaaactgattgcgttctacgatgacaacggtatctcaatcgatggtcacgttgaaggctggttcactgacgacaccgcaatgcgtttcgaagcttacggctggcacgttattcgcgacatcgacggtcatgacgcggcatccatcaaacgcgcagtagaagaagcgcgcgcagtgactgacaaaccgtccctgctgatgtgcaaaaccatcatcggtttcggttccccgaacaaagccggtacccacgactcccacggtgcgccgctgggcgacgctgaaattgccctgacccgcgaacagctgggctggaaatacgcgccgttcgaaatcccgtctgaaatctatgctcagtgggatgcgaaagaagcaggccaggcgaaagaatctgcatggaatgagaagtttgcggcttacgcgaaagcttatccgcaggaagcggctgaatttacccgccgtatgaaaggcgaaatgccgtctgacttcgacgccaaagcgaaagagtttatcgctaaactgcaggctaatccggcgaaaatcgccagccgtaaagcgtcgcagaatgctatcgaagcgttcggcccgctgttgcctgaattcctcggcggctctgctgacctggcaccgtctaacctgaccctgtggtctggttctaaagcaatcaacgaagatgctgcaggtaactacatccactacggtgttcgcgagttcggtatgaccgcgattgctaacggtatctccctgcacggtggtttcctgccgtacacctccaccttcctgatgttcgtggaatacgcacgtaacgccgtacgtatggctgcgctgatgaaacagcgtcaggtgatggtttacacccacgactccatcggtctgggcgaagatggcccgactcaccagccggttgagcaggtcgcttctctgcgcgtgaccccgaacatgtctacatggcgtccgtgtgaccaggttgaatccgcggtcgcgtggaaatacggcgttgagcgtcaggacggcccgactgcgcttatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaattagcatttcgggtaaaaaggtcgcttcggcgaccttttttattaccttgatatgtccgtttgcggacaagcaatagataaagcgtgttgtagatcacaaatatttatatgcaataaatatcaattatgtaatatgcatcacgatatgcgtattgacatttgttgttataactataactcaatgttatataagaaattaacttgaaataattaacaaacaaaggagttacagttagaaattgtaggagagatctcgtttttcgcgacaatctggcgtttttcttgctaattccaggattaatccgttcatagtgtaaaaccccgtttacacattctgacggaagatatagattggaagtattgcattcactaagataagtatggcaacactggaacagacatgaattatcagaacgacgatttacgcatcaaagaaatcaaagagttacttcctcctgtcgcattgctggaaaaattccccgctactgaaaatgccgcgaatacggttgcccatgcccgaaaagcgatccataagatcctgaaaggtaatgatgatcgcctgttggttgtgattggcccatgctcaattcatgatcctgtcgcggcaaaagagtatgccactcgcttgctggcgctgcgtgaagagctgaaagatgagctggaaatcgtaatgcgcgtctattttgaaaagccgcgtaccacggtgggctggaaagggctgattaacgatccgcatatggataatagcttccagatcaacgacggtctgcgtatagcccgtaaattgctgcttgatattaacgacagcggtctgccagcggcaggtgagtttctcgatatgatcaccccacaatatctcgctgacctgatgagctggggcgcaattggcgcacgtaccaccgaatcgcaggtgcaccgcgaactggcatcagggctttcttgtccggtcggcttcaaaaatggcaccgacggtacgattaaagtggctatcgatgccattaatgccgccggtgcgccgcactgcttcctgtccgtaacgaaatgggggcattcggcgattgtgaataccagcggtaacggcgattgccatatcattctgcgcggcggtaaagagcctaactacagcgcgaagcacgttgctgaagtgaaagaagggctgaacaaagcaggcctgccagcacaggtgatgatcgatttcagccatgctaactcgtccaaacaattcaaaaagcagatggatgtttgtgctgacgtttgccagcagattgccggtggcgaaaaggccattattggcgtgatggtggaaagccatctggtggaaggcaatcagagcctcgagagcggggagccgctggcctacggtaagagcatcaccgatgcctgcatcggctgggaagataccgatgctctgttacgtcaactggcgaatgcagtaaaagcgcgtcgcgggtaaggtttaattgtcggatgcgccgtcagagtggcgtatccgatgaatcaccacaggcctgataagtcgcgcagcgtcgcatcaggcaatgtgctccattgttagcaacaaaaaagccgactcacttgcagtcggctttctcattttaaacgaatgacgtttacttcgctttaccctggtttgcaaccgccgctgctttcgctctcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaggaggaggaatcgccatggagaggattgtcgttactctcggggaacgtagttacccaattaccatcgcatctggtttgtttaatgaaccagcttcattcttaccgctgaaatcgggcgagcaggtcatgttggtcaccaacgaaaccctggctcctctgtatctcgataaggtccgcggcgtacttgaacaggcgggtgttaacgtcgatagcgttatcctccctgacggcgagcagtataaaagcctggctgtactcgataccgtctttacggcgttgttacaaaagccgcatggtcgcgatactacgctggtggcgcttggcggcggcgtagtgggcgatctgaccggcttcgcggcggcgagttatcagcgcggtgttcgtttcattcaagtcccgacgacgttactgtcgcaggtcgattcctccgttggcggcaaaactgcggtcaaccatcccctcggtaaaaacatgattggcgcgttctaccagcctgcttcagtggtggtggatctcgactgtctgaaaacgcttcccccgcgtgagttagcgtcggggctggcagaagtcatcaaatacggcattattcttgacggtgcgtttttcaactggctggaagagaatctggatgcgttgttgcgtctggacggtccggcaatggcgtactgtattcgccgttgttgtgaactgaaggcagaagttgtcgccgccgacgagcgcgaaaccgggttacgtgctttactgaatctgggacacacctttggtcatgccattgaagctgaaatggggtatggcaattggttacatggtgaagcggtcgctgcgggtatggtgatggcggcgcggacgtcggaacgtctcgggcagtttagttctgccgaaacgcagcgtattataaccctgctcacgcgggctgggttaccggtcaatgggccgcgcgaaatgtccgcgcaggcgtatttaccgcatatgctgcgtgacaagaaagtccttgcgggagagatgcgcttaattcttccgttggcaattggtaagagtgaagttcgcagcggcgtttcgcacgagcttgttcttaacgccattgccgattgtcaatcagcgtaatcatcgttcatgcctgatgccgctatgtaggccggataaggcgttcacgccgcatccggcaaccgatgcctgatgcgacgcggtcgcgtcttatcaggcctacaggtcgatgccgatatgtacatcgtattcggcaattaatacatagca

SEQ ID NO: 34 - DNA, 7769 bp, Escherichia coli
  DNA sequence of the plasmid YEP24 (1)..(7769)
gaattctgaaccagtcctaaaacgagtaaataggaccggcaattcttcaagcaataaacaggaataccaattattaaaagataacttagtcagatcgtacaataaagctttgaagaaaaatgcgccttattcaatctttgctataaaaaatggcccaaaatctcacattggaagacatttgatgacctcatttctttcaatgaagggcctaacggagttgactaatgttgtgggaaattggagcgataagcgtgcttctgccgtggccaggacaacgtatactcatcagataacagcaatacctgatcactacttcgcactagtttctcggtactatgcatatgatccaatatcaaaggaaatgatagcattgaaggatgagactaatccaattgaggagtggcagcatatagaacagctaaagggtagtgctgaaggaagcatacgataccccgcatggaatgggataatatcacaggaggtactagactacctttcatcctacataaatagacgcatataagtacgcatttaagcataaacacgcactatgccgttcttctcatgtatatatatatacaggcaacacgcagatataggtgcgacgtgaacagtgagctgtatgtgcgcagctcgcgttgcattttcggaagcgctcgttttcggaaacgctttgaagttcctattccgaagttcctattctctagaaagtataggaacttcagagcgcttttgaaaaccaaaagcgctctgaagacgcactttcaaaaaaccaaaaacgcaccggactgtaacgagctactaaaatattgcgaataccgcttccacaaacattgctcaaaagtatctctttgctatatatctctgtgctatatccctatataacctacccatccacctttcgctccttgaacttgcatctaaactcgacctctacattttttatgtttatctctagtattactctttagacaaaaaaattgtagtaagaactattcatagagtgaatcgaaaacaatacgaaaatgtaaacatttcctatacgtagtatatagagacaaaatagaagaaaccgttcataattttctgaccaatgaagaatcatcaacgctatcactttctgttcacaaagtatgcgcaatccacatcggtatagaatataatcggggatgcctttatcttgaaaaaatgcacccgcagcttcgctagtaatcagtaaacgcgggaagtggagtcaggctttttttatggaagagaaaatagacaccaaagtagccttcttctaaccttaacggacctacagtgcaaaaagttatcaagagactgcattatagagcgcacaaaggagaaaaaaagtaatctaagatgctttgttagaaaaatagcgctctcgggatgcatttttgtagaacaaaaaagaagtatagattctttgttggtaaaatagcgctctcgcgttgcatttctgttctgtaaaaatgcagctcagattctttgtttgaaaaattagcgctctcgcgttgcatttttgttttacaaaaatgaagcacagattcttcgttggtaaaatagcgctttcgcgttgcatttctgttctgtaaaaatgcagctcagattctttgtttgaaaaattagcgctctcgcgttgcatttttgttctacaaaatgaagcacagatgcttcgttaacaaagatatgctattgaagtgcaagatggaaacgcagaaaatgaaccggggatgcgacgtgcaagattacctatgcaatagatgcaatagtttctccaggaaccgaaatacatacattgtcttccgtaaagcgctagactatatattattatacaggttcaaatatactatctgtttcagggaaaactcccaggttcggatgttcaaaattcaatgatgggtaacaagtacgatcgtaaatctgtaaaacagtttgtcggatattaggctgtatctcctcaaagcgtattcgaatatcattgagaagctgcagcgtcacatcggataataatgatggcagccattgtagaagtgccttttgcatttctagtctctttctcggtctagctagttttactacatcgcgaagatagaatcttagatcacactgcctttgctgagctggatcaatagagtaacaaaagagtggtaaggcctcgttaaaggacaaggacctgagcggaagtgtatcgtacagtagacggagtatactagtatagtctatagtccgtggaattctcatgtttgacagcttatcatcgataagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaattcccccttacacggaggcatcaagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaa

SEQ ID NO: 35 - DNA, 2000 bp, Escherichia coli
  DNA sequence of the deleted aroE region (1)..(2000)
actacgtccgtcctctgaaatcttcagcggatggacatatcgtcaaagttctggaggggcaggtttgccctgcatgtggcgcaaatctggtattacgccagggacgctttggtatgtttattggttgcattaactaccctgaatgcgaacataccgaacttatcgataaaccggacgaaacagcaattacatgcccccaatgtcggacgggccatctggtccagcgccgctcccgttatggcaaaacatttcactcttgtgatcgctacccggagtgtcaatttgccattaacttcaaacccatagctggagaatgccctgagtgtcattatccgctactcatcgaaaagaaaaccgcgcagggtgtaaaacacttttgtgccagtaaacaatgtggaaagccggtttcggcggaataataacgtgaataataacctgcaaagagacgctatcgcagctgcgatagatgttctcaatgaagaacgtgtcatcgcctatccaacggaagccgttttcggtgttgggtgcgatcctgatagcgaaacagcagtgatgcgactgttggagttaaaacagcgtccggttgataaggggctgattttaatcgcagcaaattacgagcagcttaaaccctatattgatgacaccatgttgactgacgtgcagcgtgaaaccattttttcccgctggccaggtcctgtcacctttgtctttcccgcgcctgcgacaacaccgcgctggttgacgggccgctttgattcgcttgctgtacgagtcaccgaccatccgttggtggttgctttgtgccaggcttatggtaaaccgctggtttctaccagtgccaacttgagtggattgccaccttgtcgaacagtagacgaagttcgcgcacaatttggcgcggcgttcccggttgtgcctggtgaaacgggggggcgtttaaatccttcagaaatccgcgatgccctgacgggtgaactgtttcgacaggggtaacataatcaggccatccagtttccggacagggaagagtgggacgagaataaaaaatgtgtatgttttcccgctctcgtgaatggtatgcaactgacatgcgcgatctctggcgagagtctggcgtatcgctttactggagatacgccagaacagtggttagcgagttttcgtcagcatcgctgggacctggaagaagaagcggaaaacttaattcaggaacaaagtgaagatgatcaaggctgggtctggttaccctgatccagatattcgtccttccatttcacgtaattattcgcggaatagcgtaacccagccttctcttcatcacttaacgggcggatctgtttgacggggctaccgagatacagatatccgctctccagccgtttattttgtgggaccagactacccgcaccaatcatcacatcatcttctactattgcgccatcaagtaaaattgagcccatcccaaccaaaactcgattgccaatggtgcagccgtggagcatcaccttgtgaccaacagtgacatcttcgccaatggttaatgggttgccatctgggttgtacgaggatttatgagtgacatgcaacatactgccatcctggatattggtgcgtgctccgatctgtacataatgtacatctccacgaatcacaacgagcggccagatccccacatcatcagccagacgaacgtcaccaatcacgacactgctatcgtcgatcattacgcgctgaccgatttgtggaaaaagatcgcggtatgggcgtaaaacatcagacatacttacctcagcaataaatgatttactaatgactttgggggcattattggccttgtgcaagtcttttagtatgcaaaaaagcaccgttttgtgtgcgattgcagcaaaaagggtgaaaaaacaacaaacagaaaaaaagatcaaaaaaatacttgtgcaaaaaattgggatccctataatgcgcctccgttgagacgacaacgtgaaacacttcac

SEQ ID NO: 36 - DNA, 2460 bp, Escherichia coli
  DNA sequence of the integrated cassette  ack::P15aroB (1)..(2460)
gatcggcggcataaaacggatcgcataacgcgtcatcttgataacgcgattttcgacaaagaccggggcaaggcgtttttccagcggccacgtctttgagtaatgctgtccccggcgaaacaagctaaaaaaattaacagaacgattatccggcgttgacatgcttcacctcaacttcacatataaagattcaaaaatttgtgcaaattcacaactcagcgggacaacgttcaaaacattttgtcttccatacccactatcaggtatcctttagcagcctgaaggcctaagtagtacatattcattgagtcgtcaaattcatatacattatgccattggctgaaaattacgcaaaatggcatagactcaagatatttcttccatcatgcaaaaaaaaatttgcagtgcatgatgttaatcataaatgtcggtgtcatcatgcgctacgctctatggctccctgacgtttttttagccacgtatcaattataggtacttccctcgaggctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctagaggaggaggaatcgccatggagaggattgtcgttactctcggggaacgtagttacccaattaccatcgcatctggtttgtttaatgaaccagcttcattcttaccgctgaaatcgggcgagcaggtcatgttggtcaccaacgaaaccctggctcctctgtatctcgataaggtccgcggcgtacttgaacaggcgggtgttaacgtcgatagcgttatcctccctgacggcgagcagtataaaagcctggctgtactcgataccgtctttacggcgttgttacaaaagccgcatggtcgcgatactacgctggtggcgcttggcggcggcgtagtgggcgatctgaccggcttcgcggcggcgagttatcagcgcggtgttcgtttcattcaagtcccgacgacgttactgtcgcaggtcgattcctccgttggcggcaaaactgcggtcaaccatcccctcggtaaaaacatgattggcgcgttctaccagcctgcttcagtggtggtggatctcgactgtctgaaaacgcttcccccgcgtgagttagcgtcggggctggcagaagtcatcaaatacggcattattcttgacggtgcgtttttcaactggctggaagagaatctggatgcgttgttgcgtctggacggtccggcaatggcgtactgtattcgccgttgttgtgaactgaaggcagaagttgtcgccgccgacgagcgcgaaaccgggttacgtgctttactgaatctgggacacacctttggtcatgccattgaagctgaaatggggtatggcaattggttacatggtgaagcggtcgctgcgggtatggtgatggcggcgcggacgtcggaacgtctcgggcagtttagttctgccgaaacgcagcgtattataaccctgctcacgcgggctgggttaccggtcaatgggccgcgcgaaatgtccgcgcaggcgtatttaccgcatatgctgcgtgacaagaaagtccttgcgggagagatgcgcttaattcttccgttggcaattggtaagagtgaagttcgcagcggcgtttcgcacgagcttgttcttaacgccattgccgattgtcaatcagcgtaatcatcgttcatgcctgatgccgctatgtaggccggataaggcgttcacgccgcatccggcaaccgatgcctgatgcgacgcggtcgcgtcttatcaggcctacaggtcgatgccgatatgtacatcgtattcggcaattaatacatagcatttcacaccgccagctcagctggcggtgctgttttgtaacccgccaaatcggcggtaacgaaagaggataaaccgtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcact

SEQ ID NO: 37 - DNA, 1000 bp, Escherichia coli
  gene (1)..(1000)
  gene (1)..(1000): DNA sequence of the  poxB region
gcggcccggctccgtatatggattgggtagagcaggaagtgaaagcgctcggcgtgacgcgtttctttaaagagaaattcttcaccccagtagcggaagcagcgaccagcggtctgaaattcaccaaactgcaaccggcacgagaattttacgccccggttggcaccacgctactggaggcgctggaaagcaataacgttccggttgtcgccgcctgccgtgcgggtgtttgcggctgctgtaagacaaaagtggtttccggtgaatatacggtgagcagcacaatgacgctgaccgacgccgaaatcgctgaaggttacgtactggcctgctcctgccatccgcagggggatttggttctcgcataatcgccttatgcccgatgatattcctttcatcgggctatttaaccgttagtgcctcctttctctcccatcccttccccctccgtcagatgaactaaacttgttaccgttatcacattcaggagatggagaaccaaagggtggcatttcccgtcataataaggacatgccatgattgatttacgcagtgataccgttacccgaccgagccgcgccatgctcgaagcaatgatggccgccccggttggggacgacgtttacggagacgaccctaccgttaatgctctgcaggactacgccgcagagctttccggtaaagaagccgccatttttctgccgaccggcactcaggccaacctggtcgctctgctcagtcactgcgaacgtggcgaagagtatattgtcggtcaggccgcgcataactatctgtttgaagccggtggcgcagcggtgctgggcagtattcagccgcaacccatcgacgcggctgccgacggcacgctaccgctggataaagtggcgatgaaaatcaaacccgacgatatccatttcgcccgcaccaaattactcagtctggaaaacacccacaacggcaaagtgctgccgcgtgaatacctgaaagaagcatgggaatt

SEQ ID NO: 38 - DNA, 3392 bp, Escherichia coli
  DNA sequence of the integrated cassette  poxB::tktA (1)..(3392)
gcggcccggctccgtatatggattgggtagagcaggaagtgaaagcgctcggcgtgacgcgtttctttaaagagaaattcttcaccccagtagcggaagcagcgaccagcggtctgaaattcaccaaactgcaaccggcacgagaattttacgccccggttggcaccacgctactggaggcgctggaaagcaataacgttccggttgtcgccgcctgccgtgcgggtgtttgcggctgctgtaagacaaaagtggtttccggtgaatatacggtgagcagcacaatgacgctgaccgacgccgaaatcgctgaaggttacgtactggcctgctcctgccatccgcagggggatttggttctcgcataatcgccttatgcccgatgatattcctttcatcgggctatttaaccgttagtgcctcctttctctcccatcccttccccctccgtcagatgaactaaacttgttaccgttatcacattcaggagatggagaaccaaggaaaagcgcaacggacgggcgagtagattgcgcaacatgcgagcatgatccagagatttctgaagcagcaaaaggatgttccatgtacatgacgcgcggcttgcggtaaattgttggcaaattttccggcgtagcccaaaacgcgctgtcgtcaagtcgttaagggcgtgcccttcatcatccgatctggagtcaaaatgtcctcacgtaaagagcttgccaatgctattcgtgcgctgagcatggacgcagtacagaaagccaaatccggtcacccgggtgcccctatgggtatggctgacattgccgaagtcctgtggcgtgatttcctgaaacacaacccgcagaatccgtcctgggctgaccgtgaccgcttcgtgctgtccaacggccacggctccatgctgatctacagcctgctgcacctcaccggttacgatctgccgatggaagaactgaaaaacttccgtcagctgcactctaaaactccgggccacccggaagtaggttataccgctggtgtggaaaccaccaccggtccgctgggtcagggtattgccaacgcagtcggtatggcgattgcagaaaaaacgctggcggcgcagtttaaccgtccaggtcacgacattgtcgaccactacacctacgccttcatgggcgacggctgcatgatggaaggcatctcccacgaagtttgctctctggcgggtacgctgaagctgggtaaactgattgcgttctacgatgacaacggtatctcaatcgatggtcacgttgaaggctggttcactgacgacaccgcaatgcgtttcgaagcttacggctggcacgttattcgcgacatcgacggtcatgacgcggcatccatcaaacgcgcagtagaagaagcgcgcgcagtgactgacaaaccgtccctgctgatgtgcaaaaccatcatcggtttcggttccccgaacaaagccggtacccacgactcccacggtgcgccgctgggcgacgctgaaattgccctgacccgcgaacagctgggctggaaatacgcgccgttcgaaatcccgtctgaaatctatgctcagtgggatgcgaaagaagcaggccaggcgaaagaatctgcatggaatgagaagtttgcggcttacgcgaaagcttatccgcaggaagcggctgaatttacccgccgtatgaaaggcgaaatgccgtctgacttcgacgccaaagcgaaagagtttatcgctaaactgcaggctaatccggcgaaaatcgccagccgtaaagcgtcgcagaatgctatcgaagcgttcggcccgctgttgcctgaattcctcggcggctctgctgacctggcaccgtctaacctgaccctgtggtctggttctaaagcaatcaacgaagatgctgcaggtaactacatccactacggtgttcgcgagttcggtatgaccgcgattgctaacggtatctccctgcacggtggtttcctgccgtacacctccaccttcctgatgttcgtggaatacgcacgtaacgccgtacgtatggctgcgctgatgaaacagcgtcaggtgatggtttacacccacgactccatcggtctgggcgaagatggcccgactcaccagccggttgagcaggtcgcttctctgcgcgtgaccccgaacatgtctacatggcgtccgtgtgaccaggttgaatccgcggtcgcgtggaaatacggcgttgagcgtcaggacggcccgactgcgcttatcctctcccgtcagaacctggcgcagcaggaacgaactgaagagcaactggcaaacatcgcgcgcggtggttatgtgctgaaagactgcgccggtcagccggaactgattttcatcgctaccggttcagaagttgaactggctgttgctgcctacgaaaaactgactgccgaaggcgtgaaagcgcgcgtggtgtccatgccgtctaccgacgcatttgacaagcaggatgctgcttaccgtgaatccgtactgccgaaagcggttactgcacgcgttgctgtagaagcgggtattgctgactactggtacaagtatgttggcctgaacggtgctatcgtcggtatgaccaccttcggtgaatctgctccggcagagctgctgtttgaagagttcggcttcactgttgataacgttgttgcgaaagcaaaagaactgctgtaattagcatttcgggtaaaaaggtcgcttcggcgaccttttttattaccttgatatgtccgtttgcggacaagcaatagataaagcgtgttgtagatcacaaatatttatatgcaataaatatcaattatgtaatatgcatcacgatatgcgtattgacatttgttgttataactataactcaatgttatataagaaattaaaaagggtggcatttcccgtcataataaggacatgccatgattgatttacgcagtgataccgttacccgaccgagccgcgccatgctcgaagcaatgatggccgccccggttggggacgacgtttacggagacgaccctaccgttaatgctctgcaggactacgccgcagagctttccggtaaagaagccgccatttttctgccgaccggcactcaggccaacctggtcgctctgctcagtcactgcgaacgtggcgaagagtatattgtcggtcaggccgcgcataactatctgtttgaagccggtggcgcagcggtgctgggcagtattcagccgcaacccatcgacgcggctgccgacggcacgctaccgctggataaagtggcgatgaaaatcaaacccgacgatatccatttcgcccgcaccaaattactcagtctggaaaacacccacaacggcaaagtgctgccgcgtgaatacctgaaagaagcatgggaatt

SEQ ID NO: 39 - DNA, 1045 bp, Escherichia coli
  DNA sequence of the  ptsHI region (1)..(1045)
gaagatgaaagctttaccaacaagaatattgtggttattctaccatcatcgggtgagcgttatttaagcaccgcattgtttgccgatctcttcactgagaaagaattgcaacagtaatgccagcttgttaaaaatgcgtaaaaaagcacctttttaggtgcttttttgtggcctgcttcaaactttcgcccctcctggcattgattcagcctgtcggaactggtatttaaccagactaattattttgatgcgcgaaattaatcgttacaggaaaagccaaagctgaatcgattttatgatttggttcaattcttcctttagcggcataatgtttaatgacgtacgaaacgtcagcggtcaacacccgccagcaatggactgtattgcgctcttcgtgcgtcgcgtctgttaaaaactggcgctaacaatacaggctaaagtcgaaccgccaggctagactttagttccacaacactaaacctataagttggggaaatacaatgttccagcaagaagttaccattaccgctccgacaatctgctaatccacgagatgcggcccaatttactgcttaggagaagatcatgggtttgttcgataaactgaaatctctggtttccgacgacaagaaggataccggaactattgagatcattgctccgctctctggcgagatcgtcaatatcgaagacgtgccggatgtcgtttttgcggaaaaaatcgttggtgatggtattgctatcaaaccaacgggtaacaaaatggtcgcgccagtagacggcaccattggtaaaatctttgaaaccaaccacgcattctctatcgaatctgatagcggcgttgaactgttcgtccacttcggtatcgacaccgttgaactgaaaggcgaaggcttcaagcgtattgctgaagaaggtcagcgcgtgaaagttggcgatactgtcattgaatttgatctgccgctgctggaagagaaagccaagtctaccctgactccggttgttatctccaacatggacgaaatcaaagaactgatcaaactgt

SEQ ID NO: 40 - DNA, 4595 bp, Escherichia coli
  DNA sequence of the integrated cassette  tdc::glf-glk (1)..(4595)
ctgatttctttgtcgctgatcccttactggaactctgcagttatcgaccaggttgacctcggttcgctgtcgttaaccggtcatgacggtatcctgatcactgtctggctggggatttccatcatggttttctcctttaacttctcgccaatcgtctcttccttcgtggtttctaaacgtgaagagtatgagaaagacttcggtcgcgacttcaccgaacgtaaatgttcccaaatcatttctcgtgccagcatgctgatggttgcagtggtgatgttctttgcctttagctgcctgtttactctgtctccggccaacatggcggaagccaaagcgcagaatattccagtgctttcttatctggctaaccactttgcgtccatgaccggtaccaaaacaacgttcgcgattacactggaatatgcggcttccatcatcgcactcgtggctatcttcaaatctttcttcggtcactatctggggacgctggaaggcttgaatggtctgattctgaagttcggttataaaggtgacaaaaccaaagtgtcgctgggtaaactgaatactctcagcatgatcttcatcatgggctccacctgggttgttgcctacgccaacccgaacatcctcgacctgattgaagccatgggcgcaccgattatcgcatccctgctgtgcctgttgccgatgtatgccatccgtaaagcgccgtctctggcgaaataccgtggtcgtctggataacgtgtttgttaccgtgattggtctgctgaccatcctgaacatcgtatacaaactgttttaatccgtaactcaggatgagaaaagagatgaatgaatttccggttgttttggttattaactgtggttcgtcttcgattaagttttccgtactcgatgccagcgactgtgaagtattaatgtcaggtattgccgacggtattaactcggaaaatgcattcttatccgtaaatgggggagagccagcaccgctggctcaccacagctacgaaggtgcattgaaggcaattgcatttgaactggaaaaacggagtttaaatgacagcgtggccttaattggtgcaaaagtggctgtgactgtaaaaagaaatcgaaaaagaccgttttgtgtgaaaacggtctttttgtttccttttaaccaactgccataactcgaggcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctctagagggagaggaatcgccatgagttctgaaagtagtcagggtctagtcacgcgactagccctaatcgctgctataggcggcttgcttttcggttacgattcagcggttatcgctgcaatcggtacaccggttgatatccattttattgcccctcgtcacctgtctgctacggctgcggcttccctttctgggatggtcgttgttgctgttttggtcggttgtgttaccggttctttgctgtctggctggattggtattcgcttcggtcgtcgcggcggattgttgatgagttccatttgtttcgtcgccgccggttttggtgctgcgttaaccgaaaaattatttggaaccggtggttcggctttacaaattttttgctttttccggtttcttgccggtttaggtatcggtgtcgtttcaaccttgaccccaacctatattgctgaaattgctccgccagacaaacgtggtcagatggtttctggtcagcagatggccattgtgacgggtgctttaaccggttatatctttacctggttactggctcatttcggttctatcgattgggttaatgccagtggttggtgctggtctccggcttcagaaggcctgatcggtattgccttcttattgctgctgttaaccgcaccggatacgccgcattggttggtgatgaagggacgtcattccgaggctagcaaaatccttgctcgtctggaaccgcaagccgatcctaatctgacgattcaaaagattaaagctggctttgataaagccatggacaaaagcagcgcaggtttgtttgcttttggtatcaccgttgtttttgccggtgtatccgttgctgccttccagcagttagtcggtattaacgccgtgctgtattatgcaccgcagatgttccagaatttaggttttggagctgatacggcattattgcagaccatctctatcggtgttgtgaacttcatcttcaccatgattgcttcccgtgttgttgaccgcttcggccgtaaacctctgcttatttggggtgctctcggtatggctgcaatgatggctgttttaggctgctgtttctggttcaaagtcggtggtgttttgcctttggcttctgtgcttctttatattgcagtctttggtatgtcatggggccctgtctgctgggttgttctgtcagaaatgttcccgagttccatcaagggcgcagctatgcctatcgctgttaccggacaatggttagctaatatcttggttaacttcctgtttaaggttgccgatggttctccagcattgaatcagactttcaaccacggtttctcctatctcgttttcgcagcattaagtatcttaggtggcttgattgttgctcgcttcgtgccggaaaccaaaggtcggagcctggatgaaatcgaggagatgtggcgctcccagaagtagttaaacttgctttggctgaatccttttgtcttttttagataagtcttaaccaattatactttttgtttacaacgatggtataaagcgggcggacttattttacctgttgggtagccttctgatttcagaaaggaattattatggaaattgttgcgattgacatcggtggaacgcatgcgcgtttctctattgcggaagtaagcaatggtcgggttctttctcttggagaagaaacaacttttaaaacggcagaacatgctagcttgcagttagcttgggaacgtttcggtgaaaaactgggtcgtcctctgccacgtgccgcagctattgcatgggctggcccggttcatggtgaagttttaaaacttaccaataacccttgggtattaagaccagctactctgaatgaaaagctggacatcgatacgcatgttctgatcaatgacttcggcgcggttgcccacgcggttgcgcatatggattcttcttatctggatcatatttgtggtcctgatgaagcgcttcctagcgatggtgttatcactattcttggtccgggaacgggcttgggtgttgcccatctgttgcggactgaaggccgttatttcgtcatcgaaactgaaggcggtcatatcgactttgctccgcttgacagacttgaagacaaaattctggcacgtttacgtgaacgtttccgccgcgtttctatcgaacgcattatttctggcccgggtcttggtaatatctacgaagcactggctgccattgaaggcgttccgttcagcttgctggatgatattaaattatggcagatggctttggaaggtaaagacaaccttgctgaagccgctttggatcgcttctgcttgagccttggcgctatcgctggtgatcttgctttggcacagggtcgaaccagtgttgttattggcggtggtgtcggtcttcgtatcgcttcccatttgccagaatctggtttccgtcagcgctttgtttcaaaaggacgctttgaacgcgtcatgtccaagattccggttaagttgattacttatccgcagcctggactgttgggtgcgcagctgcctatgccaacaaatattctgaagttgaataatattttttaatattatgaactgaatttaagaggctgccttccgataaaatcgggaggtggccttttttatattttttactaaaaaatgaagacaaaaaagtcttaagtaagaataatattattattaacttttgatatattttgtattagtggatccgccctcccgctggaaattgaagccatcgcagtacgtagtgcgtaaagcctcgtgagcgggacggtcgtaaggtcgttccgctccacttcactgaacggcaatccgagggtgtggatatgattagtgcattcgatattttcaaaattgggattggtccctccagttcgcataccgtggggccaatgaatgccggaaaaagttttattgatcggctggaaagtagcggcttattaaccgcgacgagccatattgtggtcgatctgtacgggtcgttgtcactgacgggcaaaggccatgccacggatgtcgccatcatcatgggactggcaggaaacagtccgcaggatgttgtcattgatgagatccctgcatttatagagttagtaacgcgcagcgggcggctgccagtggcatctggtgcgcatattgttgattttcctgtagcaaagaacattatcttccatcccgaaatgttgcctcgccatgagaacggaatgcggatcactgcctggaagggacaggaagagctattaagtaaaac

SEQ ID NO: 41 - DNA, 1069 bp, Escherichai coli
  DNA sequence of the  galP region (1)..(1069)
actttggtcgtgaacatttcccgtgggaaaaaaccgacaaagcgcagctgctgcgcgatgctgccggtctgaagtaatctttcttcacctgcgttcaaaggccagcctcgcgctggcctttttcttttggataggcgttcacgccgcatccggcaaaaaaaccgcccgcacaataacatcattcttcctgatcacgtttcaccgcagattatcatcacaactgaaaccgattacaccaaccacaacagacaaagatttgtaatattttcatattattattcggttttcacagttgttacatttcttttcagtaaagtcttaattgcagataacagcgtttaatctatgatgatataactcaattattttcatgcacttaaatcataactaagataaatgttagtgtaagcgattacactgatgtgatttgcttcacatctttttacgtcgtactcacctatcttaattcacaataaaaaataaccatattggagggcatcatgcctgacgctaaaaaacaggggcggtcaaacaaggcaatgacgtttgaaataggcgctcacgattaatctccccaagcttcctcccatcgcggaggaagccacctcttgcagtcatcttttcttcgctctatcctctgccgctatgaaaacatcccgtctccctatcgccatccaacaggccgttatgcgtcgcctgcgggaaaaactcgcccaggccaacctgaagctagggcgtaactacccggagccaaaactctcttacacccagcgcggaacctccgccggaacggcctggctggaaagctatgaaattcgcctcaatcccgttttgctgttggaaaacagtgaagcttttattgaagaagtggtaccgcacgaactggcacatttgctggtatggaaacatttcggccgcgtagcgccacatggcaaagagtggaagtggatgatggaaaacgtgctgggtgttcccgcccgtcgtacgcatcagttcgaactgcaatccgtgcgtcgcaacaccttcccctaccgctgcaagtgccaggagcatcagcttaccgtacgcc

SEQ ID NO: 42 - DNA, 6100 bp, Escherichia coli
  MYR352  adhE::P15-catAX , PR-aroY, P26-quiC (1)..(6100)
ttgattttcataggttaagcaaatcatcaccgcactgactatactctcgtattcgagcagatgatttactaaaaaagtttaacattatcaggagagcattagcttgctattgacgacagctatggttcactgtccaccaaccaaaactgtgctcagtaccgccaatatttctcccttgaggggtacaaagaggtgtccctagaagagatccacgctgtgtaaaaattttacaaaaaggtattgactttccctacagggtgtgtaataatttaattacaggcgggggcaaccccgcctgttctgcagaggaggaatatagccatggaagtgaaaatcttcaacacccaggatgttcaggattttctgcgtgttgcaagcggtctggaacaagagggtggtaatccgcgtgttaaacaaattattcatcgtgttctgagcgacctgtataaagcaattgaagatctgaatatcaccagcgacgaatattgggcaggcgttgcatatctgaatcagctgggtgcaaatcaagaagcaggtctgctgagtccgggtctgggttttgatcattatctggatatgcgtatggatgcagaagatgcagcactgggtattgaaaatgcaacaccgcgtaccattgaaggtccgctgtatgttgcgggtgcaccggaaagcgttggttatgcacgcatggatgatggtagcgatccgaatggtcataccctgattctgcatggcaccatttttgatgcagatggtaaaccgctgccgaatgcaaaagttgaaatttggcatgcaaacaccaaaggcttttatagccattttgatccgaccggtgaacagcaggcctttaatatgcgtcgtagcattattaccgatgagaatggtcagtatcgtgttcgtaccattctgcctgccggttatggttgtcctccggaaggtccgacccagcaactgctgaaccaactgggtcgtcatggtaatcgtccggcacatattcattattttgttagcgcagatggtcaccgtaaactgaccacccagattaatgttgccggtgatccgtatacctatgatgattttgcatatgccacccgtgaaggtctggttgttgatgcagttgaacataccgatccggaagcaattaaagccaatgatgtggaaggtccttttgccgaaatggtgtttgatctgaaactgacccgtctggttgatggtgttgataatcaggttgtggatcgtccgcgtctggcagtttaatacaccaaaatggttcaaaattatcaggcgagtgatcatgatcactggcctgtttttatttcagggaagggtggagacaattacgtggataatcagatcatccaagaaaccgtggataaaattctgagcgttctgccgaatcaggcaggtcagctggcacgtctggtgcgtctgatgcaatttgcatgcgatccgaccattaccgttattggcaaatataaccatggtaaaagccgtctgctgaatgaactgattggcaccgatatctttagcgttgcagataaacgtgaaaccattcagctggccgaacataaacaggatcaggttcgttggctggatgcacctggtctggatgccgatgttgcagcagttgatgatcgtcatgcatttgaagcagtttggacccaggcagatattcgtctgtttgttcatagcgttcgtgaaggtgaactggatgcaaccgaacaccatctgctgcaacagctgattgaagatgccgatcatagccgtcgtcagaccattctggttctgacccagattgatcagattccggatcagaccatcctgacacagattaaaaccagcattgcacagcaggttccgaaactggatatttgggcagttagcgcaacccgtcatcgtcagggcattgaaaacggtaaaaccctgctgatcgaaaaaagcggtattggtgcactgcgccataccctggaacaggcactggcacaggtgccgagcgcacgtacctatgaaaaaaatcgtctgctgtcagatctgcaccatcagctgaaacaactgctgctggatcagaaacatgttctgcaacaactgcaacagacacagcaacagcagctgcatgattttgataccggtctgattaacattctggacaaaattcgtgttgatctggaaccgattgtgaatattgatggtcaggatcaagcactgaatccggatagctttgcaaccatgtttaaaaacaccgcagcaaaacagcagcgtgccaaagttcagattgcatatagccgtgcatgcattgaaatcaacagccatctgattcgccatggtgttgttggtctgcctgcggaacagcagaccaccattaaaagcattgataccgtgattgttgccgtgtttggtatcagcgttaaatttcgtgatcagctgcgtgccctgttttataccgataccgaacgtcagcgtctgcaacgtgaatttcgtttctattttgaaaaaagtgccggtcgcatgattctggcagcaaaaattgaacagaccatgcgtcagcagggctgtattcagaatgccatgatggcactgcaacaaatggaaagcgcagcataaaaacacggacgccgcaaacggcgtccgaatttcttggtcgaccgttaaatctatcaccgcaagggataaatatctaacaccgtgcgtgttgactattttacctctggcggtgataatggttgcatgtactaatctagataaggaatatagccatgaccgcaccgattcaggatctgcgtgatgcaattgccctgctgcaacagcatgataatcagtatctggaaaccgatcatccggttgatccgaatgcagaactggcaggcgtttatcgtcatattggtgccggtggcaccgttaaacgtccgacccgtattggtccggcaatgatgtttaataacattaaaggttatccgcacagccgtattctggttggtatgcatgcaagccgtcagcgtgcagcactgctgctgggttgtgaagcaagtcagctggcactggaagttggtaaagcagttaaaaaaccggttgcaccggtggttgttccggcaagcagcgcaccgtgtcaagagcagatttttctggcagatgatccggattttgatctgcgtaccctgctgcctgcacataccaataccccgattgatgcaggtccgtttttttgtctgggtctggccctggcaagcgatccggtggatgcaagcctgaccgatgttaccattcatcgtctgtgtgttcagggtcgtgatgaactgagcatgttcctggcagcaggtcgccatattgaagtttttcgtcagaaagcagaagcagcaggtaaaccgctgccgattaccattaatatgggtctggacccagcaatctatattggcgcatgttttgaagcaccgaccaccccgtttggttataatgaactgggtgttgccggtgcactgcgtcagcgtccggttgaactggttcagggtgttagcgttccggaaaaagcaattgcacgtgccgaaattgttattgaaggtgaactgctgcctggtgttcgtgttcgtgaagatcagcataccaattcaggtcatgcaatgccggaatttccgggttattgtggtggtgcaaatccgagcctgccggttattaaagttaaagccgttaccatgcgcaataacgcaattctgcaaaccctggttggtccgggtgaagaacataccaccctggcaggtctgccgaccgaagcaagcatttggaatgcagttgaagcagcaattccgggttttctgcaaaatgtttatgcccataccgcaggcggtggtaaatttctgggtattctgcaagtgaaaaaacgtcagcctgccgatgaaggtcgtcagggtcaggcagccctgctggcgctggcaacctatagcgaactgaaaaatatcattctggtggatgaggatgtggacatttttgatagtgatgatattctgtgggcaatgaccacccgtatgcagggtgatgttagcattaccaccattccgggtattcgcggtcatcagctggacccgagccagacaccggaatattcaccgagcattcgtggtaatggtattagctgcaaaaccatctttgattgtaccgttccgtgggcactgaaaagccattttgaacgtgcaccgtttgcagatgttgatccgcgtccgtttgcacctgaatattttgcacgtctggaaaaaaatcagggcagcgcaaaataagctaataacaggcctgctggtaatcgcaggaatttttatttggatggatccgcctacctagcttccaagaaagatatcctaacagcacaagagcggaaagatgttttgttctacatccagaacaacctctgctaaaattcctgaaaaattttgcaaaaagttgttgactttatctacaaggtgtggtataataatcttaacaacagcaggacgctcccgggttgaggaaaacctaatgaaactgaccagcctgcgtgttagcctgctggcactgggtctggttaccagcggttttgcagcagcagaaacctataccgttgatcgttatcaggatgatagcgaaaaaggtagcctgcgttgggcaattgaacagagcaatgcaaatagcgcacaagaaaaccagattctgattcaggcagttggtaaagcaccgtatgttatcaaagttgataaaccgctgcctccgattaaaagcagcgttaaaatcattggcaccgagtgggataaaaccggtgaatttattgcaattgatggcagcaactatatcaaaggcgaaggtgaaaaagcatgtccgggtgcaaatccgggtcagtatggcaccaatgttcgtaccatgaccctgcctggtctggttctgcaagatgttaatggtgttaccctgaaaggtctggatgttcatcgtttttgtattggtgttctggttaatcgcagcagcaataacctgattcagcataatcgtatcagcaacaattatggtggtgccggtgttatgattaccggtgatgatggtaaaggtaatccgaccagcaccaccaccaataataacaaagttctggataacgtgttcatcgataatggtgatggtctggaactgacccgtggtgcagcatttaatctgattgcaaataacctgtttaccagcacaaaagccaatccggaaccgagccagggtattgaaattctgtggggtaatgataatgccgtggtgggtaacaaattcgaaaactattcagatggcctgcaaatcaattggggtaaacgtaactatatcgcctataacgaactgaccaataacagcctgggtttcaatctgacaggtgatggtaacattttcgacagcaataaagtgcatggtaaccgtattggtattgccattcgtagtgaaaaagatgccaatgcacgtattaccctgaccaaaaatcagatttgggataacggcaaagatatcaaacgttgtgaagccggtggtagctgtgttccgaatcagcgtctgggtgcaattgtttttggtgttccggcactggaacatgaaggttttgttggtagccgtggcggtggtgttgttattgaaccggcaaaactgcaaaaaacctgcacccagccgaaccagcagaattgtaatgcaattcctaatcagggtattcaggcaccgaaactgacagttagcaaaaaacagctgaccgttgaagttaaaggcacccctaatcagcgttataatgtggaattttttggcaatcgtaatgccagcagcagcgaagcagaacagtatctgggtagcattgttgttgttaccgatcatcagggtctggcaaaagcaaattgggctccgaaagttagcatgccgagcgttaccgcaaatgtgacagatcatctgggtgcgaccagcgaactgagcagcgcagttaaaatgcgttaaatgcatgcgcgccgcgttcgcgcggcgcttttttttggtactcagtagcgctgtctggcaacataaacggccccttctgggcaatgccgatcagttaaggattagttgaccgatccttaaactgaggcactataacggcttc

(19) United States
(12) Patent Application Publication
(10) Pub. No.: US 2015/0044755 A1
(43) Pub. Date: Feb. 12, 2015

(54) PRODUCTION OF MUCONIC ACID FROM GENETICALLY ENGINEERED MICROORGANISMS

(71) Applicant: MYRIANT CORPORATION, Woburn, MA (US)

(72) Inventors: R. Rogers Yocum, Lexington, MA (US); Wei Gong, Woburn, MA (US); Sudhanshu Dole, North Andover, MA (US); Ryan Sillers, Winchester, MA (US); Meghal Gandhi, Fremont, MA (US); Janice G. Pero, Lexington, MA (US)

(73) Assignee: MYRIANT CORPORATION, Quincy, MA (US)

(21) Appl. No.: 14/375,071

(22) PCT Filed: Jan. 29, 2013
(86) PCT No.: PCT/US13/23690

§ 371 Date: Jul. 28, 2014

Related U.S. Application Data
(60) Provisional application No. 61/632,777, filed on Jan. 30, 2012.

(51) Int. Cl. C12N 1/20

(52) U.S. Cl. CPC C12N 1/20
USPC 435/252.33

(57) ABSTRACT

This present invention is in the field of producing renewable chemical feedstocks using biocatalysts that have been genetically engineered to increase their ability to convert renewable carbon resources into useful compounds. More specifically, the present invention provides a process for producing muconic acid form renewable carbon resources using a genetically modified organism.

### CROSS-REFERENCE TO RELATED APPLICATIONS

This application claims the priority of the U.S. Provisional Application Ser. No. 61/632,777, filed on Jan. 30, 2012.

### FIELD OF THE INVENTION

The present invention is in the field of producing renewable chemical feedstocks using biocatalysts that have been genetically engineered to increase their ability to convert renewable carbon resources into useful compounds. More specifically, the present invention provides a process for producing muconic acid isomers from renewable carbon resources using genetically modified biocatalysts.

### BACKGROUND OF THE INVENTION

Adipic acid is a large volume chemical used in the manufacture of Nylon 66. Adipic acid is currently made from petrochemicals, but the synthesis is not environmentally friendly (Niu et al., 2002). Alternatively, adipic acid can be made from any of the three isomers of muconic acid (cis,cis; cis,trans; trans,trans) by chemical hydrogenation. It would be desirable to produce muconic acid from renewable resources by fermentation with a microorganism, followed by hydrogenation to adipic acid, since such a route to adipic acid would be more environmentally friendly than the traditional petrochemical route.

Current efforts towards microbial production of muconic acid can be grouped under three categories namely: (1) An aromatic degradation pathway for muconic acid production, in which various aromatic compounds are fed, and the benzene ring portion of aromatic compounds are oxidatively cleaved open; (2) A muconate buildup pathway, in which the muconic acid backbone is built up from various C2, C3, C4, compounds or lysine; and (3) An aromatic amino acid biosynthetic muconic acid pathway, in which muconic acid is built from 3-dehydroshikimate, an intermediate in the aromatic amino acid biosynthetic pathway in many organisms.

Many microorganisms are capable of degrading aromatic compounds containing a benzene ring, such as phenol, catechol, and benzoic acid, using pathways that cleave the aromatic ring to give terminal or intermediate compounds that are non-aromatic compounds such as cis,cis-muconic acid, or 3-carboxy-cis,cis-muconic acid (Niu et al., 2002; Perez-Pantoja et al., 2008). In the past, a number of groups have attempted to exploit this ability of microbes in the production of cis,cis-muconic acid at the industrial level (Mizuno et al, 1988; Yoshikawa et al, 1990; Choi et al, 1997). In the late 1980s, Celgene Corporation of USA and Mitsubishi Chemical Industries of Japan were active in developing a process for manufacturing muconic acid from toluene and benzoic acid respectively, as evidenced by a number of granted United States and Japanese patents in this area.

A number of microbial organisms have been reported to produce cis,cis-muconic acid using toluene, benzoic acid, benzene or catechol. For example, with catechol as the source of carbon, cis,cis-muconic acid production can be achieved with an almost 100% molar conversion yield using a recombinant E. coli cells expressing the catA gene, which encodes the Pseudomonas putida mt-2 catechol 1,2-dioxygenase responsible for catalyzing ortho-cleavage of catechol, as biocatalyst (Kaneko et al, 2011). Bioreactors for the continuous production of muconic acid using this system have been described.

This approach of microbial production of cis,cis-muconic acid using cyclic C6 carbon compounds never became a commercial reality; however, there has been continuous academic interest in understanding the functioning of the enzymes in the degradation pathway for muconic acid production in microbes.

A recently, published international patent application (WO 2011/017560) claims biocatalysts having a muconate pathway and a method for producing muconic acid using these biocatalysts. In brief, this published patent application discloses four different pathways for producing muconic acid. The first pathway for muconic acid production starts with succinyl-CoA and acetyl-CoA. The second pathway for muconic acid production begins with pyruvate and malonate semialdehyde. The third pathway for muconic acid production starts with pyruvate and succinic semialdehyde. The fourth pathway for muconic acid production starts with lysine. All these pathways for muconic acid production proposed in this patent application are based on computer modeling and it is yet to be seen whether such biocatalysts can ever be created with commercially acceptable productivity and yield for muconic acid.

A fermentation route to cis,cis-muconic acid using a genetically engineered E. coli system has been described in the scientific literature (Niu et al., 2002) and in patent literature (U.S. Pat. No. 5,616,496; U.S. Pat. No. 5,487,987; WO 2011/085311 A1), but the prior art processes need to be substantially improved with respect to titer, yield, and suitability for large scale commercial production in order to be economically attractive. There have been two reports of Saccharomyces cerevisiae yeasts that were genetically engineered to produce cis,cis-muconic acid from glucose, but the published titers were only 1.5 mg/l and 140 mg/l (Weber et al., 2012; Curran et al., 2012). At these titers, neither of these yeast processes would be attractive for commercial production. This present invention describes a process for producing cis,cis-muconic acid or cis,trans-muconic acid, by fermentation that is substantially improved compared to the published processes well known in the art with respect to suitability for large scale commercial production.

One of the objectives of the invention disclosed herein is to produce cis,cis-muconic acid by fermentation of a microorganism starting from a renewable, non-aromatic carbon source, such as a sugar or other simple carbon compound that can be derived from photosynthetic plants, using a genetically engineered organism that is suitable for large scale commercial production.

In 2002, Niu et al published a “benzene free” route to produce adipic acid that used a fermentation process to produce cis,cis-muconic acid, and then a catalytic chemical process to convert the cis,cis-muconic acid to adipic acid. This process was patented, but as far as the present inventors know, this process has not been commercialized, (U.S. Pat. No. 5,487,987; U.S. Pat. No. 5,616,496). The fermentation portion of this published process used genetically engineered strains of E. coli, the best of which was named WN1/pWN2.248. The pathway uses a portion of the native aromatic amino acid biosynthetic pathway (also known as the “shikimic acid pathway”, the “shikimate pathway”, the “chorismate pathway”, the “common aromatic pathway”, the “central aromatic pathway”, or simply the “aromatic pathway”, part of which is illustrated in FIG. 1. In this specification, any biochemical step downstream from a carbon source fed to an organism, for example glucose, and which leads directly or indirectly to chorismate, is considered to be part of the shikimic acid pathway, including, for example, the steps catalyzed by, Glf, Glk, Zwf, TktA, TalB, and Pps. In addition, the published process uses three heterologous enzymes that convert 3-dehydroshikimate (an intermediate in the aromatic pathway) to cis,cis-muconic acid through the intermediates protocatechuate and catechol. The engineered host strain is a derivative of E. coli K-12 that has a genotype of aroE353, serA::(aroB, aroZ), lacZ::(tktA, aroZ), where aroB encodes 3-dehydroquinate synthase (hereinafter named AroB), tktA encodes transketolase, and aroZ is a heterologous gene from Klebsiella pneumoniae that encodes 3-dehydroshikimate dehydratase (hereinafter called “AroZ”). The engineered strain contains a multicopy plasmid, pWN2.248, which is derived from pBR322 and contains gene cassettes for expressing catA, catX, aroY, aroF (feedback resistant), serA, lacI^{q}, and ampicillin resistance. The heterologous genes catA and catX were from Acinetobacter calcoaceticus and encode catechol 1,2-dioxygenase (hereinafter named “CatA”), and a protein (“CatX”) of unknown function that might enhance CatA activity. In the literature, the catX gene is also called “orf1” (Neidle and Ornston, 1986). In this patent specification, we shall refer to the catA plus catX gene pair from Acinetobacter as “catAX” The heterologous gene aroY was from Klebseilla pneumoniae and encodes protocatechuate decarboxylase (hereinafter called “AroY”).

A more recent patent application, related to the issued patents described above, has been published (WO 2011/085311 A1). In this application, the same strain as mentioned above, WN1/pWN2.248, was used to produce cis,cis-muconic acid, which was then isomerized into cis,trans-muconic acid,

However, the strain WN1/pWN2.248 is not well suited for large scale commercial production, so there is a need for a much improved process. The present invention provides improved biocatalysts for the fermentative production of cis,cis-muconic acid.

The process described in WO 2011/085311 A1 has several other features that make it impractical for implementation on a large commercial scale. The aroE353 mutation that was included in the biocatalyst WN1/pWN2.248 used in the fermentative production of cis,cis-muconic acid functions to block flow of carbon into the lower part of the shikimate pathway so as to maximize flow into the desired pathway to cis,cis-muconic acid. However the aroE mutation is a “null” mutation (a mutation that renders the gene inactive for all practical purposes), which has the effect of turning the strain into an auxotroph for the aromatic amino acids (phenylalanine, tyrosine, and tryptophan) and aromatic vitamins or vitamin-like intermediates made from the shikimate pathway (p-hydroxy benzoic acid, p-amino benzoic acid, and 2,3-dihydroxy benzoic acid). The aromatic amino acids are relatively expensive, and their requirement would add a large burden to the cost of producing cis,cis-muconic acid. Thus, there is also a need for a process that does not require these expensive nutrients to be added to the growth medium.

Yet another problem associated with the currently available biocatalyst for the production of cis,cis-muconic acid is related to the need for maintaining a multicopy plasmid to express some of the necessary genes (Niu et al., 2002). Multicopy plasmids are often too unstable to be used in large scale industrial processes. Moreover, at least one of the genes on the plasmids is expressed from a promoter, P_{tac}, that requires either isopropylthiogalactoside (IPTG) or lactose for induction, and those two chemicals are too expensive to allow an economically attractive process. Thus there is a need for more stable strains that have expression cassettes stably integrated into the chromosome of the production strain, and there is a need for high levels of expression from constitutive promoters so as to alleviate the need for chemical inducers for the promoters.

### SUMMARY OF THE INVENTION

This present invention provides genetically engineered microorganisms that produce cis,cis-muconic acid starting from non-aromatic carbon sources. The genetically engineered microorganisms do not need to contain any exogenous plasmids in order to produce muconic acid, although they have certain exogenous or heterologous genes necessary to achieve the desired phenotype. The exogenous genes introduced into the microorganisms are stably integrated into the chromosomal DNA. As a result of this chromosomal DNA integration of the exogenous genes, the need for the use of antibiotics or other selective methods to maintain the plasmids carrying exogenous DNA is totally eliminated. In addition, strong promoters that do not require chemical inducers are used to express genes necessary for the pathway from carbon source, such as glucose, to cis,cis-muconic acid.

In one embodiment of the present invention, the activity of a negative regulator of aromatic amino acid biosynthesis is genetically manipulated. In one aspect of the present invention, the activity of the negative regulator TyrR is substantially reduced by means of controlling the expression of the tyrR gene coding for the TyrR protein. In another aspect of the present invention, the activity of the negative regulator TyrR is totally eliminated by means of deleting or inactivating the tyrR gene from the chromosomal DNA of the microorganism.

In another embodiment of the present invention the feedback inhibition of certain enzymes in the aromatic amino acid pathway due to certain metabolites is overcome through genetic manipulations. In most wild type E. coli strains, deoxyarabino-heptulosonate 7-phosphate (DAHP) synthase occurs as three different isozymes which are known to be encoded by three different genes namely aroG, aroF and aroH. The proteins encoded by each of these three genes are subjected to feedback inhibition by one or more metabolites of shikimic acid pathway responsible for aromatic amino acid biosynthesis. In one aspect of the present invention, the wild type aroG gene is replaced by a modified aroG gene which codes for an AroG protein that is resistant to feedback inhibition by one or more metabolites of the aromatic amino acid pathway within the microbial cell. In another aspect of the present invention, the wild type aroF gene is replaced by an aroF gene which codes for an AroF protein that is resistant to feedback inhibition by one or more metabolites of the aromatic amino acid pathway within the microbial cell. In yet another aspect of the present invention, the wild type aroH gene is replaced by an aroH gene which codes for an AroH protein that is resistant to feedback inhibition by one or more metabolites of the aromatic amino acid pathway within the microbial cell. In yet another embodiment of the present invention the biocatalyst selected for the commercial production of cis,cis-muconic acid may have more than one feedback resistant isozyme for deoxyarabino-heptulosonate 7-phosphate (DAHP) synthase.

In another embodiment of the present invention, the activity of one or more of the enzymes involved in the flow of carbon through the aromatic amino acid pathway within the microbial cell is enhanced. In one aspect of the present invention, the enhancement of the activity of one or more enzymes involved in the operation of an aromatic amino acid pathway and/or a muconic acid pathway is accomplished through genetic manipulation. In a preferred embodiment of the present invention, the expression of one or more of the genes coding for enzymes or proteins AroF, AroG, AroH, AroB, TktA, TalB, AroZ, QutC, qa-4, asbF, QuiC, AroY, Rpe, Rpi, Pps, CatA and CatX or their homologs or analogs are enhanced leading to the increased activity of said enzymes. Rpe is a ribulose-5-phosphate epimerase, Rpi is a ribulose-5-phosphate isomerase, and Pps is a phosphoenol pyruvate synthetase (Neidhardt and Curtiss, 1996). If the host strain is a yeast, for example Saccharomyces cerevisiae, or a filamentous fungus, for example, Neurospora crassa, several of the enzymes that catalyze reactions in the shikimate pathway can be combined into one large protein or polypeptide, called Aro1p, encoded by the ARO1 gene in the case of S. cerevisiae. Aro1p combines the functions of AroB, AroD, AroE, AroK (or AroL), and AroA). As such, for the purposes of this invention, Aro1p, and ARO1, or a portion thereof, can be used as a substitute, or in addition to, AroB, AroD, AroE, AroK, and/or AroA.

In yet another embodiment of the present invention, flux through erythrose-4-phosphate within the bacterial cell is enhanced by means of overexpressing enzymes in the operation of the pentose phosphate pathway. In one aspect of the present invention over production of the transaldolase enzyme coded by the talB or talA gene is engineered. In another aspect of the present invention, the expression of the genes encoding both transaldolase and transketolase enzymes are enhanced by genetic manipulations. In yet another aspect of the present invention, the expression of the genes encoding either or both ribulose-5-phosphate epimerase and ribulose-5-phosphate isomerase are enhanced by genetic manipulations.

In another embodiment of the present invention, the PEP (phosphoenol pyruvate) available for the functioning of the aromatic amino acid pathway is increased through genetic manipulation. In one aspect of the present invention, competition for the PEP pool is decreased through elimination and/or complementation of the PEP-dependent phosphotransferase system (PTS) for glucose uptake with a PEP independent system for glucose uptake. In yet another embodiment of the present invention, the availability of PEP is increased by increasing the expression of a gene that encodes a PEP synthetase, such as pps.

### BRIEF DESCRIPTION OF THE DRAWINGS

FIG. 1. Pathway for aromatic amino acid biosynthesis in E. coli.

FIG. 2. Pathway for the production of muconic acid in E. coli.

FIG. 3. Chromatograph showing standards used for HPLC analysis of total muconic acid and biochemical intermediates.

FIG. 4. Chromatograph showing standards used for HPLC analysis of muconic acid isomers.

FIG. 5. Titer for the production of DHS in the E. coli strain MYR34 transformed with plasmids pCP32AMP, pCP14 and pCP54. MYR34 strain of E. coli has a deletion in aroE gene. The plasmid pCP32AMP expresses the aroG gene coding for DAHP synthase. The plasmid pCP14 expresses the aroB gene coding for DHQ synthase. The plasmid pCP54 expresses both the aroB and aroG genes.

FIG. 6. Titer for the production of DHS in the E. coli strains MYR34 and MYR170 transformed with plasmids pCP32AMP and pCP54. The MYR34 strain of E. coli has a deletion of the aroE gene. The MYR170 strain has a deletion of the aroE gene and a second copy of the aroB gene under the control of P₁₅ promoter integrated at the ack locus of the host chromosomal DNA. The plasmid pCP32AMP expresses the aroG gene coding for DAHP synthase. The plasmid pCP54 expresses both aroB and aroG genes.

FIG. 7. Titer for the production of cis,cis-muconic acid in the E. coli strains MYR34 and MYR170 transformed with plasmid pMG37 alone or with both pMG37 and pCP32AMP plasmids. The MYR34 strain of E. coli has a deletion of the aroE gene. The MYR170 strain has a deletion of the aroE gene and a second copy of the aroB gene under the control of P₁₅ promoter integrated at the ack locus of the host chromosomal DNA. The plasmid pCP32AMP expresses the aroG gene coding for DAHP synthase. The plasmid pMG37 expresses the aroZ, aroY, and catAX, genes coding for proteins functional in the muconic acid pathway.

FIG. 8. Titer for the production of DHS in MYR170 strain of E. coli transformed with a plasmid expressing aroG gene alone (pCP32AMP) or aroG and tktA genes simultaneously (pCP50). The MYR170 strain has a deletion of the aroE gene and a second copy of the aroB gene under the control of the P₁₅ promoter integrated at the ack locus of the host chromosomal DNA.

FIG. 9. DHS yield from MYR34 and MYR170 stains of E. coli transformed with plasmids pCP32AMP and pCP50. DHS yield is calculated as grams of DHS produced per gram of glucose consumed. The plasmid pCP32AMP expresses the aroG gene while pCP50 expresses aroG and tktA. The bacterial strain MYR34 has a deletion in the aroE gene. The MYR170 strain of E. coli is derived from MYR34 and has an additional aroB gene integrated at the ack locus on the chromosomal DNA.

FIG. 10. DHS titer from MYR170 and MYR261 stains of E. coli transformed with plasmids pCP32AMP and pCP50. The plasmid pCP32AMP expresses the aroG gene while pCP50 expresses the aroB and tktA genes. The MYR170 strain has a deletion of the aroE gene and a second copy of the aroB gene under the control of Pᵢₛ promoter integrated at the ack locus of the host chromosomal DNA. MYR261 strain of E. coli is derived from the MYR170 strain of E. coli. MYR261 strain of E. coli has a second copy of the tktA gene with its native promoter integrated at the poxB locus of the chromosomal DNA.

FIG. 11. Muconic acid and acetic acid production in the E. coli strains MYR170, MYR261 and MYR305 transformed with the plasmid pCP32AMP expressing aroG coding for DAHP synthase in the shikimic acid biosynthetic pathway and plasmid pMG37 expressing aroZ, aroY and catAX genes coding for proteins functional in the muconic acid pathway. MYR170 strain has a deletion in the aroE gene and an additional copy of aroB gene under the control of P₁₅ promoter inserted at ack locus in the host chromosomal DNA. MYR261 and MYR305 are derivatives of MYR170 strain. MYR261 has an additional copy of tktA gene integrated at poxB locus on the host chromosomal DNA while MYR305 has a deletion in the poxB locus on the host chromosomal DNA.

FIG. 12. Conversion of endogenous DHS produced by E. coli strain MYR34 into muconic acid. MYR34 strain of E. coli has a deletion in the aroE gene coding of shikimate dehydrogenase. As a result there is an accumulation of DHS. When MYR34 strain is transformed with a plasmid expressing aroZ, aroY and catAX, genes coding for proteins functional in muconic acid pathway, there is conversion of DHS into muconic acid. However, no conversion of DHS into muconic acid occurs when MYR34 strain is transformed with the empty plasmid vector (pCL1921) without any exogenous genes.

FIG. 13. Comparison of aroZ homologs for their ability to divert DHS into the muconic acid pathway. Three different aroZ homologs, namely quiC from Acinetobacter sp. ADP1, asbF from Bacillus thuringiensis, and qa-4 from Neurospora crassa were cloned under the P₂₆ promoter in a low-copy plasmid which also expressed catAX and aroY genes from the P₁₅ and lambda P_{R} promoters respectively. These three different plasmid constructs were expressed in MYR34 through transformation and the amount of muconic acid produced was measured.

FIG. 14. Single copies of catAX, aroY and quiC were chromosomally integrated into MYR170 strain of E. coli (ΔaroE, Δack::P₁₅-aroB) resulting in MYR352 (SEQ ID No. 41). MYR170 was also transformed with low copy plasmid pMG37 carrying all genes necessary for the operation of muconic acid pathway leading to the MYR219 strain. Both MYR352 and MYR219 were transformed with YEp24 (medium-copy empty vector) or pCP32AMP (medium-copy aroG expressing plasmid) or pCP50 (medium-copy aroG and tktA expressing plasmid) and the amount of PCA, catechol and muconic acid produced were quantified using HPLC method.

FIG. 15. Removal of catechol accumulation in MYR352 by means of increasing the expression of catAX. MYR352 was transformed with a plasmid expressing aroY alone (pMG27) or a plasmid expressing quiC alone (pMG39) or a plasmid expressing all three muconic acid pathway genes namely catAX, aroY and quiC (pMG37) or a plasmid expressing only two genes in the muconic acid pathway namely catAX and aroY (pMG33). Over expression of catAX alone was sufficient to prevent accumulation of catechol.

FIG. 16. Growth of strains using different systems for importing glucose. Deletion of ptsHI and galP (MYR31) leads to lack of growth in minimal glucose medium, while installation of glf and glk genes (MYR217) brings back growth. Control strain MYR34 is ΔaroE, but otherwise wild type. The three aromatic amino acids and three aromatic vitamins were added to the medium to allow growth of the auxotrophic strains.

FIG. 17. DHS production in MYR34 and MYR217 strains of E. coli. When transformed with plasmids that lead to production of DHS, MYR217, which utilizes glf-glk for glucose import, produced a higher titer of DHS than transformants of MYR34, which utilizes the phosphotransferase system (PTS)

FIG. 18. Production of muconic acid by the MYR428 strain of E. coli in a 7 Liter fermentor. The MYR261 strain of E. coli with a genotype of ΔaroE ΔackA::P₁₅-aroB ΔpoxB::tktA was transformed with the plasmids pCP32AMP and pMG37 to generate the MYR428 strain of E. coli.

### DETAILED DESCRIPTION OF THE PREFERRED EMBODIMENTS

As used in this patent application, the phrase “for example” or “such as” is meant to indicate that there are more than one method, approach, solution, or composition of matter for the subject at hand, and the example given is not meant to be limiting to that example.

The term “heterologous” refers to a gene or protein that is not naturally or natively found in an organism, but which can be introduced into an organism by genetic engineering, such as by transformation, mating, or transduction. A heterologous gene can be integrated (inserted) into a chromosome or contained on a plasmid. The term “exogenous” refers to a gene or protein that has been introduced into, or altered, in an organism for the purpose of increasing, decreasing, or eliminating an activity, by genetic engineering, such as by transformation, mating, transduction, or mutagenesis. An exogenous gene or protein can be heterologous, or it can be a gene or protein that is native to the host organism, but altered by one or more methods, for example, mutation, deletion, change of promoter, change of terminator, duplication, or insertion of one or more additional copies in the chromosome or in a plasmid. Thus, for example, if a second copy of the aroB gene is inserted at a site in the chromosome that is distinct from the native site, the second copy would be exogenous.

The term “microorganism” as used in this present invention includes bacteria, archaea, yeast, algae and filamentous fungi that can be used for the commercial production of cis,cis-muconic acid through a fermentation process.

For nomenclature, a gene or coding region is usually named with lower case letters in italics, for example “aroZ”, while the enzyme or protein encoded by a gene can be named with the same letters, but with the first letter in upper case and without italics, for example “AroZ” or “Aro1p”, the latter of which is an example of the convention used in yeast for designating an enzyme or protein. The “p” is an abbreviation for protein, encoded by the designated gene. The enzyme or protein can also be referred to by a more descriptive name, for example, AroZ can also be referred to as 3-dehydroshikimate dehydratase. A gene or coding region that encodes one example of an enzyme that has a particular catalytic activity can have several different names because of historically different origins, or because the gene comes from different species. For example the gene that encodes 3-dehydroshikimate dehydratase from Bacillus thuringiensis or Bacillus anthracis can be named asbF instead of aroZ, the related gene from Aspergillus nidulans can be named qutC, the related gene from Neurospora crassa can be named qa-4, and the related gene from Acinetobacter baylyi (also known as Acinetobacter calcoaceticus and Acinetobacter Sp. ADP1) can be named quiC.

A “plasmid” means a circular or linear DNA molecule that is substantially smaller than a chromosome, separate from the chromosome or chromosomes of a microorganism, and that replicates separately from the chromosome or chromosomes. A “plasmid” can be present in about one copy per cell or in more than one copy per cell. Maintenance of a plasmid within a microbial cell in general requires an antibiotic selection, but complementation of an auxotrophy can also be used.

The term “chromosome” or “chromosomal DNA” as used in this invention in the context of a bacterial cell is a circular DNA molecule that is substantially larger than a plasmid and does not require any antibiotics selection.

An “expression cassette” means a DNA sequence that can be part of a chromosome or plasmid that contains at least a promoter and a gene or region that codes for an enzyme or other protein, such that the coding region is expressed by the promoter, and the enzyme or protein is produced by a host cell that contains the DNA sequence. An “expression cassette” can be at least partly synthetic, or constructed by genetic engineering methods, so that the coding region is expressed from a promoter that is not naturally associated with the coding region. Optionally, the “expression cassette” can contain a transcription terminator that may or may not be a terminator that is naturally associated with the coding region. An “expression cassette” can have coding regions for more than one protein, in which case it can be called an operon, or a synthetic operon.

“Overexpression” of a gene or coding region means causing the enzyme or protein encoded by that gene or coding region to be produced in a host microorganism at a level that is higher than the level found in the wild type version of the host microorganism under the same or similar growth conditions. This can be accomplished by, for example, one or more of the following methods: 1) installing a stronger promoter, 2) installing a stronger ribosome binding site, such as a DNA sequence of 5′-AGGAGG, situated about four to ten bases upstream of the translation start codon, 3) installing a terminator or a stronger terminator, 4) improving the choice of codons at one or more sites in the coding region, 5) improving the mRNA stability, and 6) increasing the copy number of the gene, either by introducing multiple copies in the chromosome or placing the cassette on a multicopy plasmid. An enzyme or protein produced from a gene that is overexpressed is said to be “overproduced”. A gene that is being “overexpressed” or a protein that is being “overproduced” can be one that is native to a host microorganism, or it can be one that has been transplanted by genetic engineering methods from a different organism into a host microorganism, in which case the enzyme or protein and the gene or coding region that encodes the enzyme or protein is called “foreign” or “heterologous”. Foreign or heterologous genes and proteins are by definition overexpressed and overproduced, since they are not present in the unengineered host organism.

A “homolog” of a first gene, DNA sequence, or protein is a second gene, DNA sequence, or protein that performs a similar biological function to that of said first gene, DNA sequence or protein, and that has at least 25% sequence identity (when comparing protein sequences or comparing the protein sequence derived from gene sequences) with said first gene or protein, as determined by the BLAST computer program for sequence comparison (Altschul et al., 1990; Altschul et al., 1997), and allowing for deletions and insertions. An example of a homolog of the E. coli aroG gene would be the aroG gene from Salmonella typhimurium.

An “analog” of a first gene, DNA sequence, or protein is a second gene, DNA sequence, or protein that performs a similar biological function to that of said first gene, DNA sequence, or protein, but where there is less than 25% sequence identity (when comparing protein sequences or comparing the protein sequence derived from gene sequences) with said first gene, DNA sequence or protein, as determined by the BLAST computer program for sequence comparison (Altschul et al., 1990; Altschul et al., 1997), and allowing for deletions and insertions. An example of an analog of the Klebsiella pneumoniae AroZ protein would be the QutC protein from Aspergillus nidulans, since both proteins are enzymes that catalyze the 3-dehydroshikimate dehydratase reaction, but there is no significant sequence homology between the two enzymes or their respective genes. A scientist knowledgeable in the art will know that many enzymes and proteins that have a particular biological function, for example DAHP synthase or 3-dehydroshikimate dehydratase, can be found in many different organisms, either as homologs or analogs, and since members of such families of enzymes or proteins share the same function, although they may be slightly or substantially different in structure, different members of the same family can in many cases be used to perform the same biological function using current methods of genetic engineering. Thus, for example, the AroZ enzyme and the QutC enzyme catalyze the same reaction, DHS dehydratase, so either one will result in production of cis,cis-muconic acid in the proper context, and the choice of which one to use ultimately can be made by choosing the one that leads to a higher titer of cis,cis-muconic acid under similar fermentation conditions.

A “non-aromatic carbon source” or a “non-aromatic compound” is a carbon-containing compound that can be used to feed a microorganism of the invention as a source of carbon and/or energy, in which the compound does not contain a six-membered ring related to benzene. Examples of non-aromatic carbon sources include glucose, xylose, lactose, glycerol, acetate, arabinose, galactose, mannose, maltose, or sucrose. An “aromatic compound” is a compound that contains one or more six-membered rings related to benzene. An example of an aromatic compound is catechol, or 1,2-dihydroxy benzene.

A “strong constitutive promoter” is a DNA sequence that typically lies upstream (to the 5′ side of a gene when depicted in the conventional 5′ to 3′ orientation), of a DNA sequence or a gene that is transcribed by an RNA polymerase, and that causes said DNA sequence or gene to be expressed by transcription by an RNA polymerase at a level that is easily detected directly or indirectly by any appropriate assay procedure. Examples of appropriate assay procedures include 1) quantitative reverse transcriptase plus PCR, 2) enzyme assay of an encoded enzyme, 3) Coomassie Blue-stained protein gel, or 4) measurable production of a metabolite that is produced indirectly as a result of said transcription, and such measurable transcription occurring regardless of the presence or absence of a protein that specifically regulates level of transcription, a metabolite, or inducer chemical. An example of a promoter that is not a “strong constitutive promoter” is the P_{lac} promoter of E. coli, since it is repressed by a repressor in the absence of lactose or the inducer IPTG. By using well known methods in the art, a “strong constitutive promoter” can be used to replace a native promoter (a promoter that is otherwise naturally existing upstream from a DNA sequence or gene), resulting in an expression cassette that can be placed either in a plasmid or chromosome and that provides a level of expression of a desired DNA sequence or gene at a level that is higher than the level from the native promoter. A strong constitutive promoter can be specific for a species or genus, but often a strong constitutive promoter from a bacterium can function well in a distantly related bacterium. For example, a promoter from Bacillus subtilis or a phage that normally grows on B. subtilis can function well in E. coli. A “strong constitutive promoter” is substantially different from inducible promoters, such as P_{tac}, which have been used in the prior art production of cis,cis-muconic acid and typically require an expensive chemical or other environmental change for the desired level of function (Niu et al., 2002). Examples of strong constitutive promoters are P₁₅, P₂₆, from Bacillus subtilis phage SP01, and coliphage lambda P_{R} (SEQ ID Nos. 1, 2, and 3).

A “muconic pathway” or “muconic acid pathway” refers to a biochemical pathway from DHS to PCA to catechol to cis,cis-muconic acid, and a “muconic pathway gene” or “muconic acid pathway gene” is a gene that encodes an enzymes that catalyzes a step in a muconic pathway, or encodes an auxiliary function that serves to enhance the activity of one of said enzymes, for example, aroZ, aroY, catA, catX, and qutC. DHS is an abbreviation for 3-dehydroshikimate, and PCA is an abbreviation for protocatechuic acid. A “muconic plasmid” is a plasmid that contains one or more muconic pathway genes.

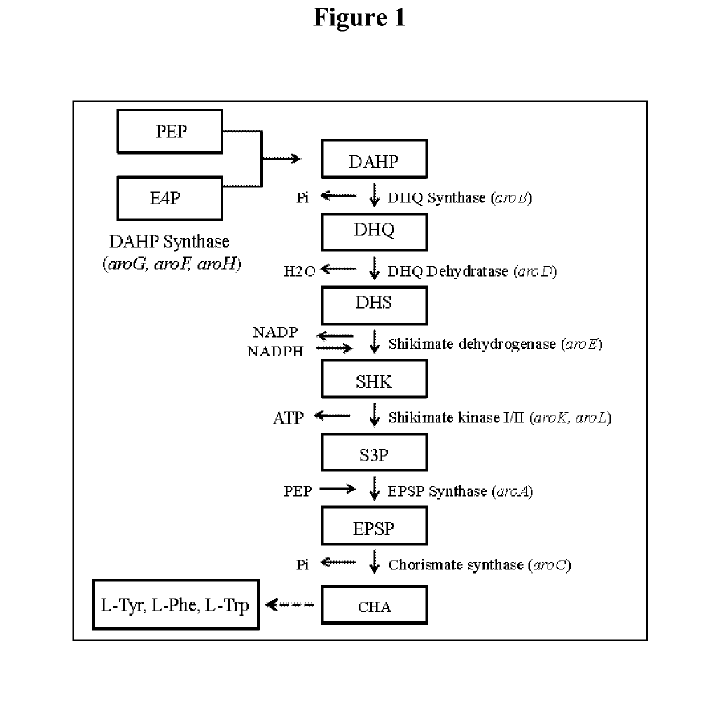

Some of the genetic manipulations used in the present inventions are centered around the common pathway for aromatic amino acid biosynthesis present in the bacterial cells as shown in FIG. 1. The common pathway for aromatic amino acid biosynthesis as depicted in FIG. 1 from DAHP synthase to chorismate synthase is also referred as the “shikimic acid pathway”.

There is a substantial volume of published work on genetic engineering of microorganisms for the production of the aromatic amino acids, phenylalanine, tyrosine, and tryptophan (U.S. Pat. No. 4,681,852, U.S. Pat. No. 4,753,883, U.S. Pat. No. 6,180,373, European Patent Application 86300748.0). The approaches include using various combinations of feedback resistant enzymes (AroF, AroG, PheA, TyrA), deregulation of repression of transcription (tyrR), increasing promoter strength (P_{tac}, P_{lac}) and increasing the copy number of one or more genes (tktA). However, many specific combinations of the above approaches were not tried, either because there were too many combinations to try without undue experimentation, or because lack of insight into what would be the best combinations. More importantly, the suitability of any of these combinations of genetic manipulations in developing a biocatalyst for the commercial production of muconic acid using renewable, non-aromatic carbon sources is not yet known.

The aromatic amino acid biosynthetic pathway is well known for many microorganisms, especially for E. coli (Neidhardt and Curtiss, 1996). In a wild type cell, the pathway is tightly regulated by both feedback inhibition and repression of transcription. The first committed step is catalyzed by deoxy-arabino-heptulosonate 7-phosphate (DAHP) synthase, of which there are three isozymes encoded by aroF, aroG, and aroH. The three isozymes, AroF, AroG, and AroH, are feedback inhibited the products of aromatic amino biosynthetic pathway namely by tyrosine, phenylalanine, and tryptophan, respectively. Feedback resistant mutants of all three are well known (Draths et al., 1992; Lutke-Eversloh and Stephanopoulos, 2007; Hu et al., 2003; Shumilin et al., 1999). One aspect of the present invention involves use of feedback resistant alleles of aroF, aroG, and aroH genes in order to express AroF, AroG and AroH enzyme proteins that are resistant to feedback inhibition by the products of aromatic amino acid biosynthetic pathway. Transcription of several of the operons involved in the aromatic pathway is regulated by either the repressor encoded by the tyrR gene or the repressor encoded by the trpR gene, or both (Neidhardt et al., 1996). Of particular importance is the negative regulation of transcription of aroG and aroF by the TyrR protein when it is bound with one or more of the aromatic amino acids. One aspect of the present invention involves the removal of negative regulation by tyrR or trpR genes by means of eliminating these genes from the chromosome of the host bacterial strain.

The subject of this invention is the creation of novel combinations of genetically engineered cassettes with novel genetically engineered elements in order to increase the fermentation parameters and suitability for large scale commercial production of a cis,cis-muconic acid producing strain. In particular, the prior art for production of cis,cis-muconic acid does not teach certain combinations of genetic elements, for example, but not limited to, various combinations of an overproduced feedback resistant AroG, an overproduced feedback resistant AroF, an overexpressed tktA, an overexpressed talA, chromosomally integrated cassettes for expressing an aroZ, aroY, and a catAX (or analogs or homologs thereof) from strong constitutive promoters, and a leaky aroE allele, which we define as a gene that encodes an AroE enzyme that confers prototrophy for the aromatic amino acids and vitamins, but without leading to significant secretion of aromatic compounds.

All specific examples of strain constructions disclosed herein are derived from a wild type E. coli C strain (ATCC 8739), or E. coli K-12 strains (YMC9 or MM294) but the genetic elements disclosed herein can be assembled in any other suitable E. coli strain, and the expression cassettes or appropriate analogs and homologs of the genetic elements disclosed herein can be assembled in any other suitable microorganism, such as other species of bacteria, archaea, yeast, algae, and filamentous fungi that can be used for the commercial production of cis,cis-muconic acid through a fermentative process.

In E. coli, the aromatic amino acid biosynthesis pathway from glucose starts with the non-oxidative branch of the pentose phosphate pathway (PPP). Four key enzymes in the non-oxidative pentose phosphate pathway are transketolase, transaldolase, ribulose-5-phosphate epimerase and ribulose-5-phosphate isomerase. These enzymes catalyze the reactions that lead to the formation of erythrose 4-phosphate (E4P) from hexose or pentose sugars. To increase the availability of E4P in E. coli, the tktA gene encoding transketolase can be overexpressed (Niu et al., 2002). Similarly, the overexpression of the transaldolase gene is also expected to increase the availability of E4P in some circumstances (Bongaerts et al., 2001). In yet another aspect of the present invention, the expression of both the transketolase and transaldolase genes are enhanced through genetic manipulations leading to an increase in the activity of transketolase and transaldolase enzymes. In yet another aspect of the invention, flux through the non-oxidative branch of the PPP is increased by overproducing ribulose-5-phosphate epimerase and ribulose-5-phosphate isomerase.

The first committed step and most tightly regulated reaction in the common aromatic amino acid pathway is the condensation of phosphoenolpyruvate (PEP) and E4P to produce deoxyarabino-heptulosonate 7-phosphate (DAHP) by DAHP synthase (encoded by aroG, aroF, and aroH). D-glucose consumed by E. coli is brought into aromatic biosynthesis partly through the PPP, and partly through glycolysis. The flow of glucose into the aromatic pathway is greatly increased when transketalose (tktA) and an isozyme of DAHP synthase (aroG) are amplified through transformation with a plasmid that increases their expression by increasing their copy number (Niu et al., 2002). In a preferred aspect of the present invention, the exogenous aroG and tktA genes are integrated into the chromosomal DNA for the purpose of amplification of activities transketolase and DAHP synthase enzymes.

In another embodiment of the present invention, the flux through PEP within the microbial cell is improved by increasing the PEP available for the synthesis of DAHP. Many genera of bacterial cells consume PEP in the transport of glucose across the cell membrane using a phosphotransferase system (PTS) in which one PEP molecule is consumed for every molecule of glucose transported across the bacterial outer membrane. By replacing or complementing the PEP-dependent PTS with a non-PEP dependent (PEP independent) glucose uptake mechanisms, it is possible to increase the pool size of the PEP available for the aromatic amino acid biosynthetic pathway within the microbial cell. For example, the PTS system for sugar uptake can be replaced or complemented by a GalP-based sugar uptake system or the sugar transporter system based on Glf/Glk proteins (Chandran et al., 2003; Yi et al., 2003). In a preferred aspect of the present invention besides deleting the PTS system for sugar uptake for the purpose of conserving PEP pool within the microbial cell, the GalP based sugar uptake system is also inactivated for the purpose of conserving ATP within the microbial cell. In a microbial cell which is defective in the functioning of both PTS system and a Gal-P based sugar uptake system (ΔAPTS/ΔgalP), the sugar uptake can be accomplished by means of introducing an exogenous gene coding for Glf, or exogenous genes encoding both Glf (glucose facilitated diffusion protein) and Glk (glucokinase) proteins. As used in the present invention, the term functional glucose-facilitated diffusion protein refers to any Glf protein as well as any other protein which is functionally equivalent to Glf and functions to transport sugars into the microbial cells by facilitated diffusion. In one aspect of the present invention, the gene coding for the glucose facilitator protein Glf is introduced into the microbial cell which is ΔAPTS/ΔgalP and the glucose transported into the microbial cell is phosphorylated by endogenous glucose kinase. In another aspect of the present invention the genes coding for both Glf and Glk proteins are introduced into a microbial cell which is ΔPTS/ΔgalP. In a preferred aspect of the present invention, the exogenous glf and glk genes introduced into the microbial cell are integrated into the host chromosomal DNA.

In another embodiment of the present invention, when the carbon source for growth and energy requires gluconeogenesis (for example if the carbon source is acetate or succinate), the PEP pool can be increased by increasing the activity of carboxylating enzymes already present within the cell, for example PEP carboxykinase, which is encoded by pck in E. coli, or by introducing an exogenous carboxylating enzyme. In a preferred embodiment, the introduced exogenous gene coding for a carboxylating enzyme is stably integrated into the host chromosome. Genes coding for the carboxylating enzyme can be derived from a variety of microbial species. The genes coding for the carboxylating enzymes can further be subjected to genetic manipulations so that the expression of the carboxylating enzyme within the biocatalyst for cis,cis-muconic acid production is significantly enhanced.

In yet another embodiment of the present invention, the PEP pool inside the microbial cell is increased by decreasing or eliminating the activity of pyruvate kinase enzymes such as PykA and PykF which use PEP as a substrate.

From DAHP, the aromatic amino acid pathway proceeds via a number of intermediates to chorismate (CHA), a branch point for the biosynthesis of three aromatic amino acids namely L-Tyrosine (L-Tyr), L-Phenylalanine (L-Phe), and L-Tryptophan (L-Trp).

In the initial stages of the common aromatic amino acid pathway, 3-dehydroquinate (DHQ) synthase (AroB) removes the phosphate group from DAHP leading to the formation of DHQ. The enzyme DHQ dehydratase (AroD) removes a water molecule from DHQ leading to the formation of 3-dehydroshikimate (DHS) which is subsequently reduced to shikimate (SHK) by shikimate dehydrogenase (AroE). Shikimate kinase I/II (AroK, AroL) phosphorylates shikimate to shikimate 3-phosphate (S3P). There is a condensation of S3P with PEP leading to the formation of 5 enolpyruvoylshikimate 3-phosphate (EPSP). The formation of EPSP is mediated by EPSP synthase (AroA). A phosphate group from EPSP is removed by chorismate synthase (AroC) leading to the formation of chorismate (CHA).

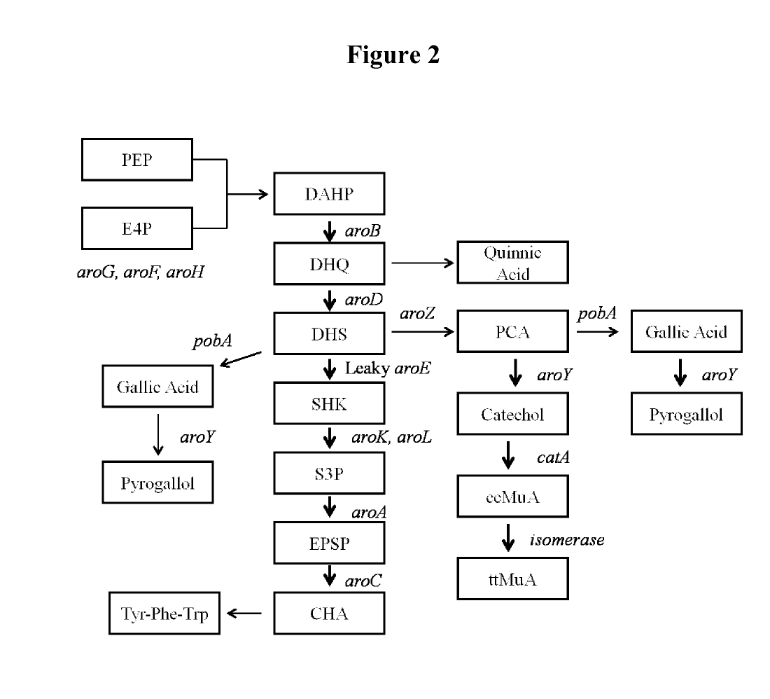

As shown in the FIG. 2, the aromatic amino acid pathway can be blocked at the level of conversion of 3-dehydroshikimate (DHS) to shikimate (SHK) due to a mutation in aroE gene leading to the accumulation of DHS (Niu et al., 2002). Introduction of an exogenous aroZ gene functions to convert DHS into protocatechuate (PCA). PCA is subsequently converted into catechol through a decarboxylation reaction mediated by an AroY enzyme. Catechol is ultimately converted into cis-cis muconic acid (ccMuA) through the action of a catA gene product. ccMuA can be acted upon by maleyl acetoacetate isomerase to yield trans-trans muconic acid (ttMuA). The biosynthetic pathway from DHS to ccMuA and ttMuA is referred as muconic acid pathway. The three different genes responsible for the conversion of DHS to ccMuA can be obtained from various microbial species and introduced into a microorganism selected for muconic acid production such as Escherichia coli. In a preferred embodiment of the present invention, the exogenous genes coding for the proteins involved in muconic acid pathway are integrated into host chromosomal DNA.

In redirecting the aromatic amino acid pathway to the production of cis,cis-muconic acid mutation of the aroE gene is critical. The aroE gene can be completely inactivated leading to a total block in the biosynthesis of aromatic amino acids as was done with the WN1/pWN2.248strain of E. coli described for the muconic acid production (Niu et al., 2002). An important drawback with the WN1/pWN2.248E. coli strain and related strains is that due to the complete inactivation of the aroE gene, this strain has become auxotrophic for the aromatic acids such as phenylalanine, tyrosine and tryptophan, and aromatic vitamins or vitamin-like compounds mentioned above. As a result, this strain during its growth for the production of cis,cis-muconic acid requires the exogenous addition of these six compounds (or a common intermediate such as shikimate), thereby adding substantially to the cost of commercial production of cis,cis-muconic acid using such a strain. A novel approach to overcome this dependency on an exogenous source of aromatic amino acids is to use a strain with a leaky mutation in aroE. The leaky aroE mutant would allow a limited flow of carbon to shikimic acid while accumulating significant amounts of DHS which is then available for the conversion into PCA by the action of an AroZ enzyme. Thus the use of a leaky mutant form of aroE would eliminate the dependence on exogenous aromatic amino acids, while still diverting the flow of carbon to cis,cis-muconic acid.

The genes coding for the synthesis of AroZ, AroY and CatA proteins essential for the conversion of DHS into cis,cis-muconic acid can be derived from many microbial species. In one embodiment, these exogenous genes are integrated into the host chromosome of the biocatalyst being developed. In a preferred embodiment, the expression of these exogenous genes within the biocatalyst is driven by a constitutive promoter without the need for any inducers.

The enzyme 3-dehydroshikimate dehydratase (AroZ; EC 4.2.1.118) is required for biosynthesis of the intermediate protocatechuate. In this specification, “AroZ” shall refer to any enzyme that catalyzes the 3-dehydroshikimate dehydratase reaction. In the prior art, this enzyme is expressed from the aroZ gene of Klebsiella pneumoniae strain A170-40 (ATCC25597) (Niu et al., 2002; Draths and Frost, 1995). However, the specific activity of AroZ varies widely among organisms, from 0.1 to 261 micromoles/min/mg (Wheeler et al, 1996; Fox et al, 2008; Pfleger et al, 2008), so a significant improvement can be had by expressing an aroZ gene also known as asbF (Fox et al, 2008; Pfleger et al, 2008), qutC (Wheeler et al, 1996), qa-4 (Rutledge, 1984), and quiC, from an organism that has a higher specific activity than K. pneumoniae, for example Acinetobacter baylyi, Aspergillus nidulans (Wheeler et al, 1996), now also known as Emericella nidulans, or Neurospora crassa (Rutledge, 1984; Stroman et al, 1978), or Podospora anserina, also known as Podospora pauciseta (Hansen et al, 2009).

As one particular example, the coding sequence for the qa-4 gene from N. crassa that encodes 3-dehydroshikimate dehydratase can be obtained by any of several well known methods, for example whole gene DNA synthesis, cDNA cloning, or by a combination of genomic DNA cloning and PCR or synthetic DNA linker synthesis. Since there are no introns in the qa-4 gene, the coding region can be obtained by PCR from genomic DNA (Rutledge, 1984). The protein sequence of the qa-4 enzyme (SEQ ID No. 4) and the DNA sequence of the native gene (SEQ ID No. 5) are known.

Alternatively, an expression cassette can be constructed for the 3-dehydroshikimate dehydratase from A. nidulans. The coding sequence for the QutC enzyme from A. nidulans can be obtained by any of several well known methods, for example whole gene DNA synthesis, cDNA cloning, or by a combination of genomic DNA cloning and PCR or synthetic DNA linker synthesis. The protein sequence of QutC (SEQ ID No. 6) and the DNA sequence of the native gene, containing no introns, are known (SEQ ID No. 7; GenBank accession number M77665.1). An expression cassette can be obtained by DNA synthesis, or by a combination of genomic cloning and PCR, so that the QutC enzyme can be produced accurately in E. coli. By expressing a coding sequence for QutC from a strong, constitutive promoter in E. coli, sufficient expression can be obtained from one or two copies of the gene integrated in the chromosome, obviating the need for maintaining more than two copies of the expression cassette on a multicopy plasmid as has been disclosed in the prior art (Niu et al., 2002), and which can lead to instability. The method described above can be used in general to obtain a DNA sequence that codes for a desired enzyme, and that coding sequence can then be used to construct an expression cassette designed to function in E. coli or another appropriate microbial host organism.

The specific activity of AroZ can also be improved by using the protein sequence from the prior art (Niu et al., 2002) but constructing an improved expression cassette, for example, in which a stronger promoter and/or ribosome binding site (RBS) has been installed in front of the coding region, as described in Example 4.

The aroZ gene encoding AroZ (3-dehydroshikimate dehydratase) from Klebsiella pneumoniae strain A170-40 can be obtained as described in the prior art. The DNA sequence of the gene and surrounding DNA can be determined by methods well known in the art. A heterologous gene of the invention such as aroZ can be built into an expression cassette using a native DNA sequence or it can be synthesized with a codon optimized sequence for the intended host organism. An aroZ gene can be cloned as described (Draths and Frost, 1995) from any other microbe that contains an active aroZ gene, for example K. pneumoniae strain 342, Acinetobacter Sp. ADP1 (Acinetobacter baylyi ADP1), Bacillus thuringiensis, Emericella nidulans, Erwinia amylovora, Pseudomonas putida W619, and many others.

The enzyme protocatechuate decarboxylase (AroY; EC 4.1.1.63) is required for biosynthesis of the intermediate catechol. In this specification, “AroY” shall refer to any enzyme that catalyzes the protocatechuate decarboxylase reaction. In the prior art, this enzyme is expressed from the aroY gene of Klebsiella pneumoniae strain A170-40 (ATCC25597) on a multicopy plasmid (Niu et al., 2002). However, once again an improvement in the process can be gained by producing enough of the enzyme from one or two copies of an expression cassette integrated in the chromosome of the host organism. This can be accomplished by obtaining an aroY gene from an organism that naturally produces an AroY enzyme that has higher specific activity than that of the K. pneumoniae AroY enzyme of the prior art, or by increasing the level of expression of the K. pneumoniae AroY by constructing an expression cassette that, for example, uses a strong constitutive promoter and/or strong RBS as described above under Example 4. The protein sequence for AroY from K. pneumoniae strain A170-40 is given in SEQ ID No. 8. The corresponding gene, aroY, can be cloned as described above (Draths and Frost, 1995), or based on the protein sequence, it can be synthesized with optimized codons for the intended host organism.

The aroY gene can be obtained from any other microorganism that contains a homolog or analog, for example, K. pneumoniae strain NCTC418 (ATCC15380), Klebsiella pneumoniae 342, and Arxula adeninivorans (Sietmann et al, 2010). The DNA sequence of the aroY gene from Klebsiella pneumoniae 342 and surrounding DNA is given as SEQ ID No. 9.

The enzyme catechol 1,2-dioxygenase (CatA; EC 1.13.11.1) is required for the last step of cis,cis-muconic acid biosynthesis. In this specification, “CatA” shall refer to any enzyme that catalyzes the catechol 1,2-dioxygenase reaction. In the prior art, this enzyme is expressed from the catA gene of Acinetobacter calcoaceticus strain ADP1 on a multicopy plasmid (Niu et al., 2002). The source strain, Acinetobacter calcoaceticus strain ADP1, apparently has been renamed Acinetobacter Sp. ADP1 and Acinetobacter baylyi ADP1 (Neidle and Ornston, 1986; Barbe et al, 2004; de Berardinis et al, 2008). In this prior art example, the catA gene was expressed from a P_{tac} promoter, which requires either lactose or IPTG (isopropylthiogalactoside) as an inducer. These compounds are too expensive for use in commercial fermentations, so again, significant improvements in the process are needed, both to eliminate the need for an expensive inducer and to create a more stable strain by integrating the expression cassette in the chromosome. This can be accomplished by constructing an expression cassette for the catA gene that uses a strong constitutive promoter, strong RBS, and/or more stable mRNA as described above in the other Examples.

The DNA sequence of the catA gene and surrounding sequences from Acinetobacter baylyi ADP1 is given in SEQ ID No. 10. The protein sequence for CatA from the same strain is given in SEQ ID No. 11. In a preferred embodiment, the expression cassette for catA contains one or two additional open reading frames that exist naturally downstream from catA, in order to increase the expression level of the catA gene (Schirmer and Hillen, 1998). Many other organisms can be a source for a catA gene, for example Pseudomonas arvilla, Pseudomonas fluorescens (Nakazawa et al, 1967; Kojima et al, 1967), Streptomyces Sp. Strain 2065 (Iwagami et al, 2000), Cupriavidus necator 335T, and many others (Perez-Pantoja et al, 2008).

In order to improve the flow of carbon towards cis,cis-muconic acid, it is necessary to block certain other pathways branching out of the aromatic amino acid pathway, besides reducing the flow of carbon from DHS to shikimate (SHK) by using a leaky aroE mutant. Some bacteria, for example in the genus Acinetobacter and Pseudomonas, contain a gene named pobA, which encodes an enzyme, p-hydroxybenzoate hydroxlase, that converts DHS into gallic acid. Although a PobA homolog or analog has not been found in E. coli, strains of E. coli engineered to produce DHS secrete measurable amounts of gallic acid (Li and Frost, 1999), so it is likely that such an enzyme does exist in E. coli. In addition, the PCA derived from DHS can be converted into gallic acid by the action of p-hydroxybenzoate hydroxlase (PobA) enzyme coded by the pobA gene. The gallic acid thus produced can be subsequently converted to pyrogallol. One way to block the carbon flow to gallic acid and pyrogallol in the biocatalyst selected for an improved cis,cis-muconic acid is to block or diminish the activity of p-hydrobenzoate hydroxlase (PobA) protein through genetic manipulations. Similarly, DHQ, the precursor to DHS can also be acted upon by shikimate dehydrogenase coded by aroE leading to the production of quinnic acid. In an embodiment of the present invention, the leaky AroE mutant enzyme is additionally selected or screened for its inability or reduced ability to convert DHQ into quinnic acid.

There are several advantages in producing trans,trans-muconic acid in place of cis,cis-muconic acid. Trans, trans-muconic acid is preferred over cis,cis-muconic acid in the Diels Alder reaction with ethylene for the production of terephthalic acid. A biocatalyst with a genetically manipulated aromatic pathway produces cis,cis-muconic acid which can be converted into trans,trans-muconic acid outside the cell using chemical conversion processes. On the other hand by means of introducing a maleylacetoacetate isomerase or similar isomerase enzyme into the biocatalyst, it is possible to convert the cis,cis-muconic acid into trans,trans-muconic acid within the bacterial biocatalyst.

The specification in this patent application provides several different aspects of invention related to the construction of a microbial strain for efficient production of muconic acid. A person skilled in the art can compile several different aspects of the present invention to construct a biocatalyst with very high efficiency for the production of muconic acid.

### EXPERIMENTAL SECTION

### General Remarks

Strain and Inoculum Preparations:

A list of the bacterial strains and the plasmids used in the present invention is provided in Table 1. All specific examples of strain constructions disclosed herein are derived from a wild type E. coli C strain (ATCC 8739), or E. coli K-12 strains (YMC9 or MM294) but the genetic elements disclosed herein can be assembled in any other suitable E. coli strain, and the expression cassettes or appropriate analogs and homologs of the genetic elements disclosed herein can be assembled in any other suitable microorganism, such as other species of bacteria, archaea, yeast, algae, and filamentous fungi that can be used for the commercial production of cis,cis-muconic acid through a fermentative process.

E. coli C is capable of fermenting 10% glucose in AM1 mineral media. AM1 medium contains 2.63 g/L (NH₄)₂HPO₄, 0.87 g/L NH₄H₂PO₄, 1.5 mM MgSO₄, 1.0 mM betaine, and 1.5 ml/L trace elements. The trace elements are prepared as a 1000× stock and contained the following components: 1.6 g/L FeCl₃, 0.2 g/L CoCl₂.6H₂O, 0.1 g/L CuCl₂, 0.2 g/L ZnCl₂.4H₂O, 0.2 g/L NaMoO₄, 0.05 g/L H₃BO₃, and 0.33 g/L MnCl₂.4H₂O. The pH of the fermentation broth is maintained at 7.0 with 1.0-10.0 M KOH or 1.0-9.0 M ammonium hydroxide.

Fermentations:

Fermentations were started by streaking on a fresh NBS-2% glucose (Jantama et al., 2008a) plate from a 40% glycerol stock of E. coli strain genetically engineered and stored in a −80° C. freezer. Plasmids, if present, are retained by including the appropriate antibiotic(s) in the agar plates and liquid media. Ampicillin (sodium salt) is used at 150 mg/L, spectinomycin HCL at 100 mg/L, tetracycline HCl at 15 mg/l, and kanamycin sulfate at 50 mg/l. After 24 to 48 hours (37° C.), a single colony is picked into 25 ml of the same medium in a shake flask. After shaking at 200 rpm at 37° C. until the cells have grown to an OD₆₀₀ of about 1.0, the culture is cooled an ice and an equal volume of sterile 80% glycerol is added. 2 ml aliquots are then frozen at −80° C. to be used as inocula for fermentations.

Cell Growth:

Cell mass was estimated by measuring the optical density at 550 nm (OD₅₅₀) or 600 nm (OD₆₀₀) using a Thermo Electronic Spectronic 20 spectrophotometer.

Analysis of Intermediates in Shikimic Acid Pathway and Muconic Acid Pathways:

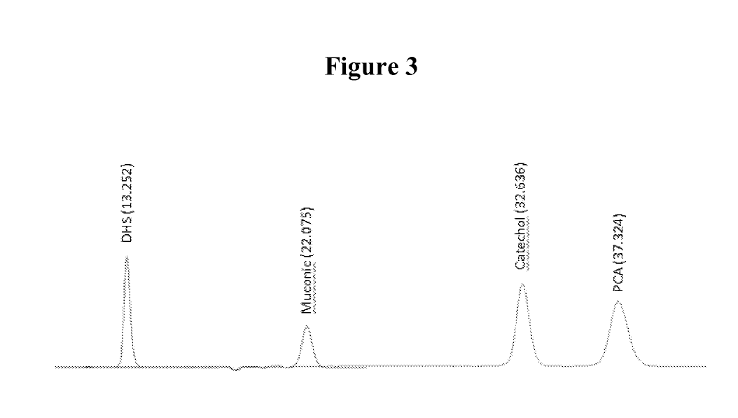

Total muconic acid produced in fermentation broths, which includes cis,cis-muconic acid and cis,trans-muconic acid, and other biochemical intermediates were assayed by HPLC with a Waters Alliance instrument, and monitoring absorbance at 210 nm or refractive index at 45° C., using standards purchased from Sigma-Aldrich. The column was a BioRad Aminex HPX-87H run at 50° C. with 8 mM sulfuric acid as the mobile phase at a flow rate of 0.6 ml/min for 40 minutes. A chromatograph of purchased standards (Sigma-Aldrich) is shown in FIG. 3. To prepare for HPLC, fermentation samples are diluted 10 or 100 fold in 0.05 M potassium phosphate buffer, pH 7.0, to preserve the cis,cis-form of muconic acid from isomerizing to the cis,trans-form.

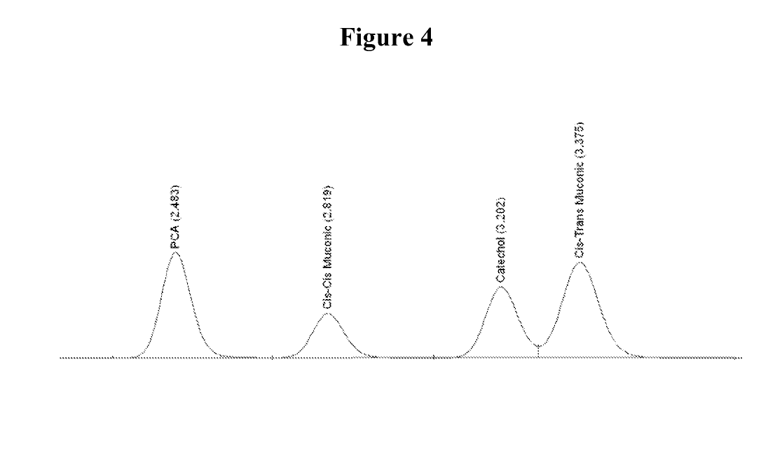

To separate the isomers of muconic acid, the samples prepared as above were run in a second HPLC system. The instrument was an Agilent 1200 HPLC, the column was an Agilent Eclipse XDB-C18, 4.6×150 mm run at 30 degrees Centigrade with a mobile phase of 50 mM KH₂PO₄ in 30% methanol adjusted to pH 3.0 with phosphoric acid. The flow rate was 1 ml/min for 4 minutes, with detection by absorbance at 278 nm. The cis,trans-muconic acid standard was created by dissolving cis,cis-muconic acid in water and allowing it to undergo spontaneous acid catalyzed isomerization for about 2 hours at room temperature, until the HPLC peak had completely shifted to a new position. The other standards were purchased from Sigma-Aldrich. A chromatograph showing standards is shown in FIG. 4.

Composition of Muconic Acid Production Medium for the Fermentation Process:

Each liter of fermentation medium contains 50 ml/L of 1M KH₂PO₄, 10 ml of 200 g/L Citric acid+25 g/L Ferric citrate, 1.2 ml of 98% Sulfuric acid, and a drop of Antifoam 204. These components were mixed with enough water to allow room for addition of other components below. After autoclaving, the following components were added: 10, 20, 30 or 40 ml of 50% glucose (to give 5, 10, 15, or 20 g/l final), 2 ml of 1M MgSO₄, 1 ml of 0.1M CaCl₂, 10 ml of 1000× Trace elements (Jantama et al. 2008a), 1, 2, 4, or 8 ml of 50 g/L Phenylalanine+50 g/L Tyrosine+50 g/L Tryptophan (to give 0.5, 0.1, 0.2, or 0.4 g/l final), 10 ml of 1 g/L p-hydroxybenzoic acid+1 g/l p-aminobenzoic acid+1 g/L 2,3-dihydroxylbenzoic acid, and, as necessary, 1 ml of 150 mg/ml Ampicillin (sodium salt) and/or 1 ml of 100 mg/ml Spectinomycin HCl.

For fed batch fermentations, the feed bottle contained 600 g/L of anhydrous glucose and 32 ml/L of 50 g/L Phenylalanine+50 g/L Tyrosine+50 g/L Tryptophan. 9M NH₄OH was used as a base to maintain the pH of the fermentation medium.

For shake flasks, NBS salts (Jantama et al. 2008a) plus 0.2 M MOPS buffer, pH 7.4 was substituted for the pre-autoclave mix described above, but the glucose and other additives were the same.

Fed-batch fermentations were performed in 7 L New Brunswick Scientific Fermentors with pH, DO, temperature, glucose, and feed rate controlled by either DCU controllers or Biocommand Software. The temperature was maintained at 37° C., the pH was maintained at 7.0 by 9N ammonium water, and the dissolved oxygen (DO) was maintained at 30% air saturation while increasing impeller's speed from 750 rpm to 1200 rpm. The initial glucose concentration in the desired medium was around 5 to 25 g/L. A glucose solution was added to the fermentor when the glucose concentration was dropped to below 5 g/L, and the feed rate of glucose was controlled by the dissolved oxygen level. The total fermentation time was 48 hrs, and the final titer was 16 g/L of muconic acid.

Construction of plasmids expressing muconic acid pathway genes:_The three heterologous genes required for conversion of DHS to muconic acid were cloned either singly or in combination into a low-copy plasmid, pCL1921 (Lerner and Inouye, 1990). The DNA sequence of pCL1921 is given in SEQ ID No. 20 in Table 3. Briefly, the coding sequences of catAX, aroY and aroZ analogs or homologs were codon-optimized for expression in E. coli and commercially synthesized (GeneArt, Invitrogen). These sequences were then PCR amplified using a forward primer carrying a unique ribosome-binding site and a reverse primer carrying a unique terminator sequence for each gene. The resulting PCR fragment was digested with restriction enzymes and cloned downstream of a unique constitutive promoter sequence by standard molecular cloning procedures. The promoter sequences were cloned by PCR amplification from source DNA sequences previously described (United States Patent Application 20090191610; U.S. Pat. No. 7,244,593) followed by restriction digestion and standard molecular cloning. The promoter-RBS-coding sequence-terminator sequence together constituted an expression cassette. Individual expression cassettes were next combined to generate plasmids expressing one, two or all three muconic acid pathway genes.

### Example 1

### Increasing Expression of aroG and aroF

The tyrR gene of E. coli can be mutated by any one of a number of well known methods, such as chemical or radiation mutagenesis and screening (for example by PCR and DNA sequencing) or selection for analog resistance (for example, resistance to 4-fluorotyrosine), transposon mutagenesis, bacteriophage Mu mutagenesis, or transformation. In a preferred embodiment, the mutation in tyrR gene is a null mutation (a mutation that leaves no detectable activity), and in a more preferable embodiment, at least a portion of the tyrR gene is deleted. This can be accomplished, for example, by using a two step transformation method using linear DNA molecules (Jantama et al, 2008a; Jantama et al, 2008b). In the first step, a cam^{R}, sacB cassette is integrated at the tyrR locus to replace most or all of tyrR open reading frame by double recombination and selecting for chloramphenicol resistance. In the second step, a linear DNA comprising a deleted version of the tyrR gene is integrated by double recombination, selecting for resistance to 5% sucrose in a rich medium such as LB. Correct deletions are identified and confirmed by diagnostic polymerase chain reaction (PCR). The purpose of deleting tyrR is to increase expression of aroG and aroF. An alternative approach that achieves a similar result is to replace the native promoter in front of aroG and/or aroF with a strong constitutive promoter and add, if necessary, a transcription terminator. More details on how this is accomplished in general are given in Example 4 below.

The latter of the two approaches described above for overcoming the repression of AroG and AroF activities by TyrR protein is preferable, since deletion of tyrR can cause unwanted overexpression of genes such as aroLM (Neidhardt and Curtiss, 1996). More detail on how this is accomplished in general is given in Example 4 below.

### Example 2

### Feedback Resistant AroG and AroF

Mutations in the aroG gene that lead to a feedback resistant AroG enzyme (3-deoxy-D-arabinoheptulosonate-7-phosphate synthase or DAHPS) are well known in the art (Shumilin et al, 1999; Kikuchi et al, 1997; Shumilin et al, 2002). Also well known are methods for creating, identifying, and characterizing such mutations (Ger et al., 1994, Hu et al., 2003). A preferable mutation is one that leads to complete resistance to inhibition by phenylalanine. Any of the known published feedback resistant mutations can be introduced into an aroG gene contained in the chromosome or on a plasmid by any of a number of well known methods, one example of which is mutagenic PCR in which the desired mutation is synthesized as part of a PCR priming oligonucleotide (Hu et al., 2003). Correct installation of the mutation is confirmed by DNA sequencing. The sequence of the wild type aroG gene from E. coli C is given in SEQ ID No. 18. A preferred mutation is a point mutation that changes amino acid 150 of AroG from proline to leucine, for example by changing codon 150 from CCA to CTA (Hu et al, 2003). In a more preferred embodiment, codon 150 is changed from CCA to CTG, which is a preferred codon in E. coli. This particular allele of aroG is preferred, since the encoded DAHP synthase is completely resistant to inhibition by phenylalanine up to 3 mM, and it has a specific activity similar to the wild type enzyme (Hu et al., 2003).

Additional feedback resistant aroG alleles can be obtained by mutagenesis and selection for resistance to one or more phenylalanine analogs, such as beta-2-thienylalanine, p-fluorophenylalanine, p-chlorophenylalanine, o-fluorophenylalanine, and o-chlorophenylalanine, followed by demonstrating that the mutation causing the resistance is linked to the aroG gene (Ger et al., 1994; U.S. Pat. No. 4,681,852). Linkage to aroG can be demonstrated directly by DNA sequencing or enzyme assay in the presence and absence of phenylalanine, (Ger et al., 1994) or indirectly by phage mediated transduction and selection for a genetic marker at or near the aroG locus that can be selected, either for or against (U.S. Pat. No. 4,681,852). Such a genetic marker can be a deletion or point mutation in the aroG gene itself, or a mutation in any suitable closely linked gene such as nadA in case of E. coli. For an example in E. coli, after mutagenesis and selection for phenylalanine analog resistance, individual mutants or pools of mutants can be used as donors for P1 mediated transduction into a naïve recipient that is deleted for all three DAHP synthase genes, aroG, aroF, and aroH, and selecting for growth on an appropriate minimal medium. The transductants will then be enriched for mutations in the desired gene(s). Alternatively, after mutagenesis and selection for analog resistance, individual mutants or pools of mutants can be used as donors for P1 mediated transduction into a naïve recipient strain that contains a null mutation in the nadA gene, again selecting for growth on an appropriate minimal medium lacking nicotinamide. Another approach is to select for resistant mutants in a strain background that contains a transposon, for example Tn10, insertion near the aroG gene, such as in the nadA gene. P1 transduction from analog resistant mutants into a strain background that does not contain said transposon and selecting for tetracycline or other appropriate antibiotic resistance will enrich for the desired aroG mutations. In all such approaches, feedback resistance is ultimately confirmed by enzyme assay and DNA sequencing of the gene. We shall refer to alleles of aroG that are resistant to feedback inhibition as aroG*.

Strain WM191 (ΔtyrR, ΔaroF) was derived from YMC9 (ATCC 33927). The two step gene replacement method (Jantama et al., 2008a) was used to install clean deletions in both tyrR and aroF, to give strain WM191. Next, a nadA::Tn10 allele was transduced in from CAG12147 (CGSC 7351, Coli Genetic Stock Center, Yale University) to give strain WM189 (ΔtyrR, ΔaroF, nadA::Tn10). Selection was on LB plus tetracycline HCl (15 mg/l). Strain RY890 (ΔtyrR::kan, aroF363) was derived from MM294 (ATCC 33625) in three steps by P1 transduction. The donor strains, in order, were JW1316-1 (CGSC 9179, Coli Genetic Stock Center, Yale University), NK6024 (CGSC 6178, Coli Genetic Stock Center, Yale University), and AB3257 (CGSC 3257, Coli Genetic Stock Center, Yale University), and the three selections, in order, were LB plus kanamycin sulfate (50 mg/l), LB plus tetracycline hydrochloride (15 mg/l), and NBS minimal glucose (Jantama et al., 2008a) with thiamine HCl (5 mg/l).

WM189 was mutagenized with UV light to about 20% survival and plated on NBS minimal glucose medium (Jantama et al., 2008a) containing o-fluorophenylalanine (1 mM), thiamine (5 mg/l), and nicotinamide (1 mM). Colonies from each of several plates were collected into separate pools, and P1vir lysates were made on each pool. These lysates were used to transduce WM191 to tetracycline resistance (15 mg/l) on LB medium, and the colonies obtained were replica plated to NBS minimal glucose medium containing o-fluorophenylalanine at 1 mM, thiamine (5 mg/l), and nicotinamide (1 mM). Colony replicas that survived both tetracycline and analog were assumed to contain a feedback resistant mutation in aroG. Eight individual colonies from 5 independent pools were chosen for DNA sequencing. The aroG coding regions were amplified by polymerase chain reaction and sequenced. The results, shown in Table 4, revealed that each of the eight strains contained a point mutation in their aroG gene. Some of the alleles were identical to published alleles, but some were novel.

A P1vir lysate from one of the pools described above was used to transduce RY890 (which has an aroG wild type allele) to tetracycline resistance and resistance to o-fluorophenylalanine (0.3 mM) by replica plating as described above. Four colonies, named RY893, RY897, RY899, and RY901, were picked for DNA sequencing (Table 4), and again, two of the alleles were identical to a published allele, but two were novel. Strain RY902, which is isogenic to the latter four strains, but contains a wild type aroG gene, was constructed as a control, by transduction from CAG12147. These five strains were grown overnight in shake flasks in 25 ml NBS minimal glucose (15 g/l) plus thiamine HCl (5 mg/l) and nicotinamide (1 mM). The resulting cells were harvested by centrifugation, resuspended to be rinsed with 10 ml water, re-centrifuged, and resuspended in 0.5 ml of 50 mM potassium phosphate, pH 7.0. The suspended cells were lysed by vortexing with three drops of chloroform, and the crude lysate was assayed for DAHP synthase activity using a method similar to a method described in the literature (Hu et al., 2003), with the following modifications. The phosphate buffer was 50 mM (final concentration), pH 7.0, the final erythrose-4-phosphate concentration was 2 mM, the final phosphoenol pyruvate concentration was 5 mM, the incubation temperature was 30° C., and the reaction was stopped at 10 minutes. We define 1 mU as the activity that produced 1 nMole of DAHP per minute per milligram protein. To test for feedback resistance, each crude lysate was assayed with or without phenylalanine at a final concentration of 18 mM. The assay results are shown in Table 5. The enzymes showed varying specific activity and resistance to phenylalanine, but all of selected mutant versions that were tested were significantly more resistant than the wild type controls.

The aroG alleles from RY893, RY899, RY901, and RY902, described above, were introduced into a muconic acid producing strain background as follows. P1vir lysates from the aroG* and aroGwt donor strains were used to transduce MYR219 (E. coli C, ΔaroE, Δack::P₁₅-aroB, pMG37) to tetracycline HCl resistance (15 mg/l), to give new strains RY903, RY909, RY911, and RY912, respectively. Each of these strains was then transduced to kanamycin sulfate resistance (50 mg/l) using a P1vir lysate of JW1316-1, to introduce the ΔtyrR::kan allele, to give strains RY913, RY919, RY921, and RY922, respectively. Spectinomycin selection was maintained throughout to maintain the muconic plasmid. The resulting four strains were grown for 48 hours at 37° C. in shake flasks in 25 ml NBS minimal medium (Jantama et al., 2008a) containing supplements of 20 g/l glucose, 0.2 M MOPS buffer, pH 7.4, nicotimamide (1 mM), phenylalanine (100 mg/l), tyrosine (100 mg/l), tryptophan (100 mg/l), p-hydroxybenzoic acid (1 mg/l), p-aminobenzoic acid (1 mg/l), 2,3-dihydroxybenzoic acid (1 mg/l), phenol red (10 mg/l), and ammonium sulfate (1 g/l). The pH was kept close to 7 as estimated by eye from the color of the phenol red, against a pH 7.0 standard, by manual addition of 1 ml aliquots of 1.0 M KOH as called for to the shake flasks. The muconic acid produced was assayed by HPLC as described above, and the results are shown in Table 6. All three strains that contain a feedback resistant aroG* allele produced more muconic acid than the isogenic strain containing the wild type aroG allele. In a separate experiment disclosed herein, strain MYR205, containing aroG on multicopy plasmid pCP32AMP, produced 1.5 g/l muconic acid in a shake flask. Thus, the inventors have shown that the combination of ΔtyrR and single copy chromosomal aroG* can perform well compared to an isogenic aroG plasmid containing strain to produce muconic acid in shake flasks. The inherent superior genetic stability of the chromosomal alleles compared to plasmid alleles, plus the alleviation of the need for a selective medium to hold in a plasmid, makes the novel strains described herein more suitable for large scale commercial fermentations. Furthermore, no chemical inducer was required for expression of muconic acid pathway genes. Thus, strains of the instant invention described above are improved over those of the prior art (Niu et al., 2002), all of which contain the gene for overexpression of DAHP synthase on an undesirable multicopy plasmid.

In a similar fashion to that described above for AroG, a mutation that leads to an AroF or AroH isozyme that is resistant to feedback inhibition by tyrosine can be installed on a plasmid or in the chromosome. A preferred mutation is a point mutation that changes amino acid 148 of AroF from proline to leucine, for example by changing codon 148 from CCG to CTG (Weaver et al., 1990), to give a gene named aroF*. Other alleles of aroF* can be isolated by resistance to tyrosine analogs (for example o-fluorotyrosine, m-fluorotyrosine, p-fluorophenylalanine, etc.) in a fashion analogous to that described above for aroG* alleles. aroF* alleles can be selected, enriched for, and transduced by linkage to a transposon or a kanamycin resistance insertion, for example in a closely linked ΔyfiR::kan as in a strain such as JW2584 (CGSC 10051, Coli Genetic Stock Center, Yale University).

### Example 3

### Deletion of aroE from Chromosomal DNA and Muconic Acid Production

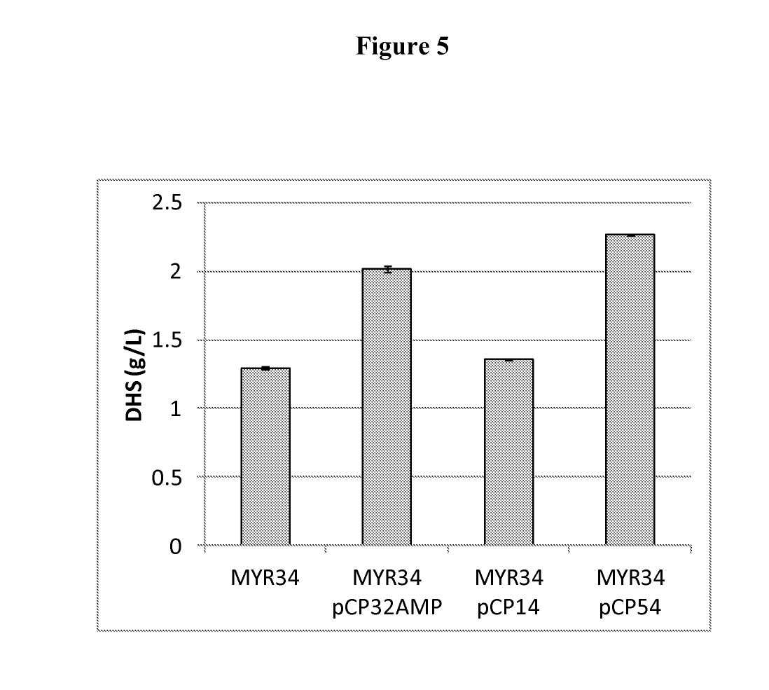

In this example the effect of overexpression of aroB and aroG on multicopy plasmids as well as the expression of genes coding for proteins functional in the muconic acid pathway was investigated. Strain MYR34 containing a deletion in the aroE gene coding for shikimate dehydrogenase was used as parent strain in these studies. The deletion of chromosomal copy of aroE was accomplished in a fashion similar to that described above in Example 1. When MYR34 was transformed with the plasmid pCP32AMP overexpressing the aroG gene coding for DAHP synthase protein functional in the shikimic acid pathway, there was a significant increase in the accumulation of DHS. When MYR34 was transformed with the plasmid expressing aroB from a constitutive promoter, no significant increase in the accumulation of DHS was noticed. However, when the E. coli strain MYR34 was transformed with the plasmid expressing both aroB and aroG genes, there was an increase in the accumulation of DHS than observed with MYR34 transformed with aroG alone thus suggesting aroB as a secondary bottleneck in DHS production (FIG. 5).

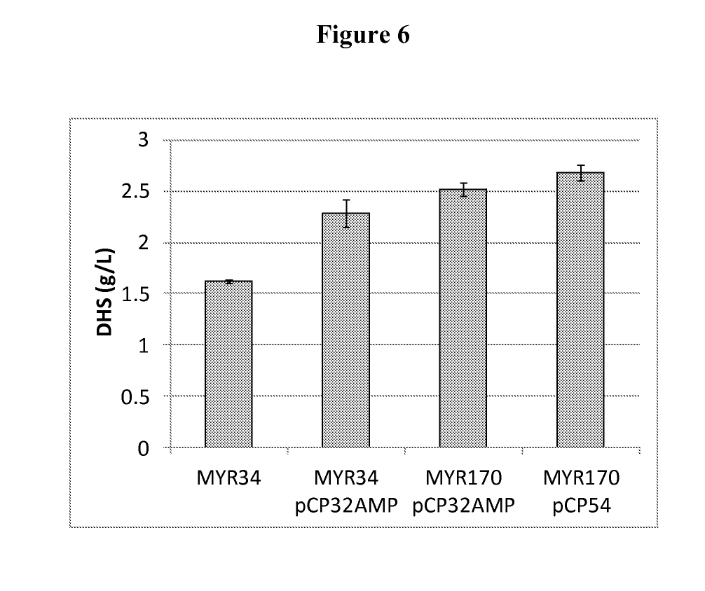

In the experiments presented in FIG. 6, the effect of an additional copy of the aroB gene integrated into the host chromosomal DNA was examined. In the E. coli strain MYR170 derived from MYR34, an additional copy of the aroB gene under the control of the P₁₅ promoter was integrated into the host chromosome at the ack locus. When MYR170 strain was transformed with the pCP32AMP plasmid, there was a slight increase in the DHS accumulation when compared to the DHS accumulation detected in the MYR34 strain transformed with the same plasmid. This slight increase in the accumulation of DHS in the MYR170 can be attributed to an additional copy of aroB gene integrated into the host chromosomal DNA. When MYR170 was transformed with pCP54 expressing both aroB and aroG genes, there was a further increase in the DHS accumulation suggesting aroB as a secondary bottleneck in DHS production.

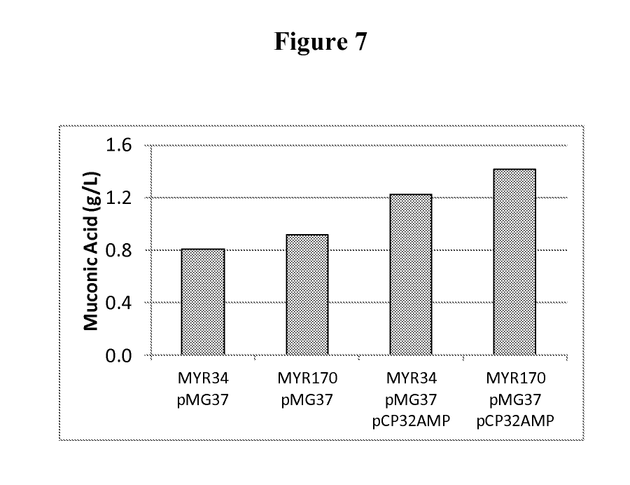

FIG. 7 provides the results on muconic acid production with the E. coli strains MYR34 and MYR170. Having established that in the aroE deletion strains MYR34 and MYR170, with overexpression of aroB and aroG genes there is an accumulation of DHS, efforts were made to see whether the expression of “muconic pathway” genes coding for the proteins functional in muconic acid production pathway would lead to conversion of DHS into cis,cis-muconic acid. In these experiments, the E. coli stains MYR34 and MYR170 were transformed either with the plasmid pMG37 alone or with both plasmids pMG37 and pCP32AMP. The plasmid pMG37 expresses aroZ, aroY and catAX genes coding for proteins functional in muconic acid pathway. The muconic acid production in both MYR34 and MYR170 increased when these bacterial strains were transformed with both the plasmids pCP32AMP and pMG37 when compared to the muconic acid production in these two strains transformed only with pMG37 plasmid suggesting that in these strains aroB expression is the bottleneck for cis,cis-muconic acid production.

### Example 4

### Overexpression of tktA

Transketolase encoded by tktA is a key enzyme in the pentose phosphate pathway and is thought to be limiting for the production of erythrose-4-phosphate, one of the key intermediates in the production of muconic acid. Overexpression of tktA, which encodes transketolase, by installing the gene with its native promoter on a multicopy plasmid (Sprenger et al, 1995, 1995a), is known to improve flux into the aromatic pathway (Draths et al., 1992). However, such plasmids are unstable, and often require antibiotic selection for maintenance. Another approach in the prior art was to add one additional copy of the tktA gene to the chromosome of the host strain (Niu et al., 2002). However, one additional copy of tktA with its native promoter is not sufficient to saturate the aromatic pathway with erythrose-4-phosphate, since its native promoter is not very close to the ideal. As such, the process needs substantial improvement.

Improved overexpression of tktA can be obtained, for example, by substituting the native tktA promoter in the chromosome with a strong constitutive promoter, for example a P₁₅ or P₂₆ promoter from Bacillus subtilis phage SPOT (SEQ ID No. 1 and SEQ ID No. 2, respectively), or the P_{R} promoter from bacteriophage lambda (SEQ ID No. 3). This is accomplished in two steps as described in Example 1, except that the cam^{R}, sacB cassette is used to replace the native chromosomal tktA promoter in the first step. In the second step, the strong constitutive promoter is installed by transforming with a linear DNA comprising the strong constitutive promoter, flanked by at least 50 bases of the 5′ end of the tktA coding region on the downstream side and at least 50 base pairs of homology just upstream of the native tktA promoter on the upstream side of the strong constitutive promoter, and selecting for sucrose resistance. Improved expression from such an expression cassette is also accomplished by increasing the stability of the mRNA that is transcribed from the expression cassette. Improvement of the mRNA stability is accomplished by adding a stem loop structure at either the 5′ end of the mRNA, the 3′ end of the mRNA, or both. A stem-loop structure is often present at the end of an mRNA that is naturally terminated by a rho-independent transcription terminator, but if it is not, then a rho-independent transcription terminator can be added to the DNA sequence by well known methods of genetic engineering (ligation, PCR, etc.). Such a terminator can be comprised of an inverted repeat of between 4 and 20 bases in each repeat, separated by a “loop” of 3 or more bases, and followed by a region of one or more bases that is enriched for T's (deoxythymidine). The inverted repeats are rich in G's and C's (deoxyguanidine and deoxycytidine). Similarly, a stem-loop can be constructed into the 5′ end of an mRNA by inserting a DNA sequence just downstream from the start point of transcription, but before the ribosome binding site, that contains a stem-loop as described above, but without the T-enriched region. An example of this is given in association with the P₁₅ promoter (SEQ ID No. 1).

In the analysis of the effect of overexpression of the tktA gene on the flow of carbon through the shikimic acid pathway, E. coli strain MYR170 was used as a parental strain. MYR170 has a deletion in the aroE gene coding for shikimate dehydrogenase enzyme and an additional copy of the aroB gene at the ack locus.

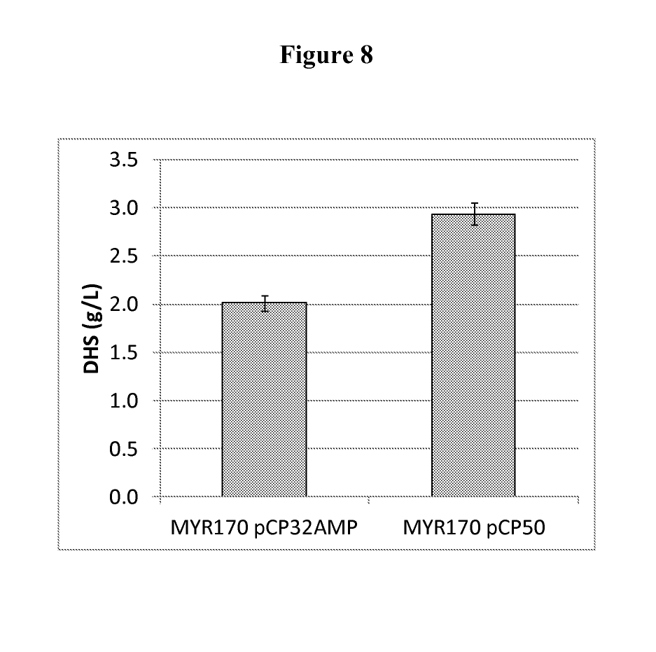

In the experiments described in the FIGS. 8, 9 and 10 two different plasmids namely pCP32AMP and pCP50 were used. The plasmid pCP32AMP expresses only the DAHP synthase aroG gene from its native promoter and the plasmid pCP50 expresses the transketolase gene tktA from its native promoter along with aroG gene. MYR170, having an aroE deletion and an additional copy of aroB gene under the control of P₁₅ promoter integrated at the ack locus of the chromosomal DNA, was transformed individually with pCP32AMP and pCP50 plasmids. As shown in FIG. 8 the DHS accumulation was increased further with the expression of aroG gene along with tktA gene when compared to the E. coli cells expressing only aroG gene.

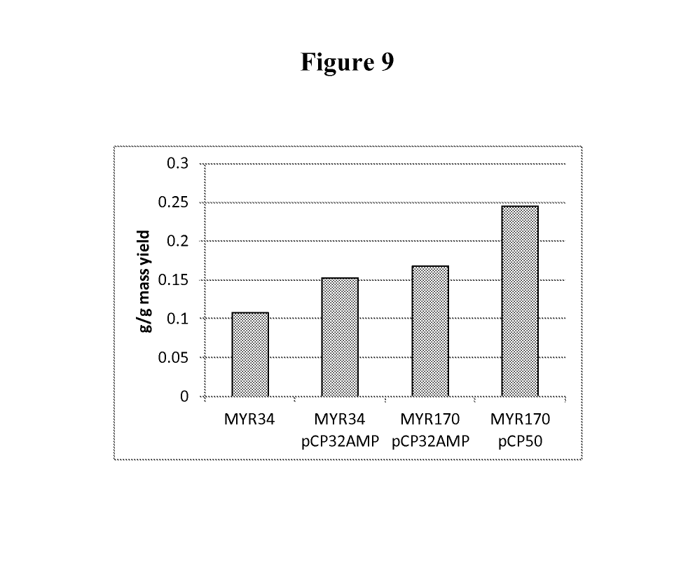

FIG. 9 provides data on the DHS yield in two different strains namely MYR34, MYR170 transformed with the plasmid pCP32AMP or pCP50. MYR34 strain having aroE gene deletion yielded 0.1 gram of DHS per gram of glucose consumed. The DHS yield in the MYR34 increased to 0.15 gram of DHS per gram of glucose consumed when this strain was transformed with the pCP32AMP plasmid with aroG gene overexpression. MYR170 has an additional copy of aroB gene inserted at the ack locus. As a result of the presence of this additional copy of the aroB gene, the yield for DHS production in the MYR170 strain transformed with pCP32AMP was slightly higher than the DHS yield noted in the MYR34 strain transformed with pCP32AMP. Thus the presence of an additional copy of aroB in MYR170 caused an increased carbon flow through shikimic acid pathway. Further increase in the DHS yield was observed when the MYR170 strain was transformed with plasmid pCP50 expressing both aroG and tktA genes. Thus the presence of additional copy of tktA accounted for an increase carbon flow through shikimic acid pathway. More specifically, the effect of presence of additional aroB and tktA genes caused an additive effect on DHS yield.

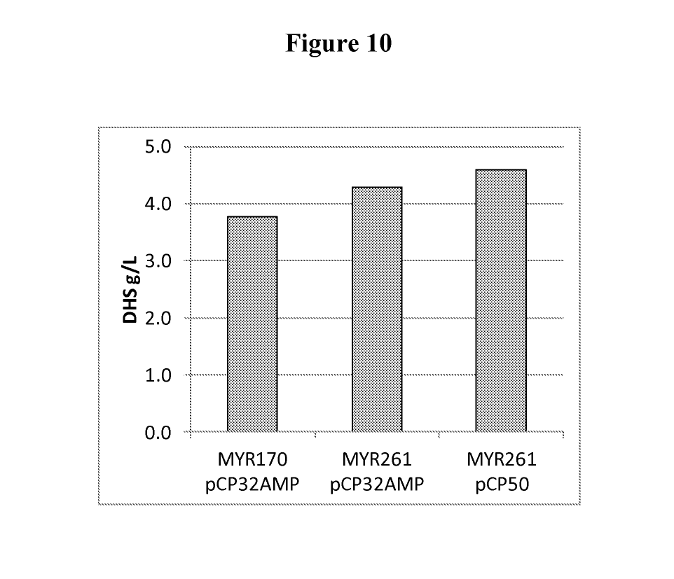

MYR261 used in the experiments described in FIG. 10 was engineered to integrate an additional copy of tktA gene into the chromosomal DNA of MYR170 at the poxB locus. The desired gene replacement (poxB::tktA) in the MYR261 strain was confirmed via PCR. MYR261 was transformed either with pCP32AMP (aroG overexpression) plasmid or pCP50 (aroG and tktA over expression) plasmid. As a control, MYR170 was transformed with pCP32AMP plasmid. As the result shown in FIG. 10 indicate, the presence of an additional copy of tktA gene in the chromosomal DNA of MYR261 increased the titer for DHS production with pCP32AMP plasmid when compared to the titer for DHS production observed in the MYR170 strain transformed with the same plasmid. Further increase in the transketolase level in the MYR261 strain when transformed with the plasmid pCP50 over expressing transketolase led to further increase in the titer for DHS production. The enzyme encoded by poxB, PoxB, or pyruvate oxidase, produces acetate as a reaction product. As such, the deletion of poxB that results from the insertion of tktA as described herein removes a potentially active pathway for acetate production. Similarly, simultaneous insertion of P₁₅aroB and deletion of ackA, which encodes AckA, or acetate kinase, as described below in Example 12 below, removes another potentially active pathway to acetate. Production of acetate is generally undesirable in fermentations (Jantama et al., 2008b). As such, these deletions can be useful for reducing acetate production.

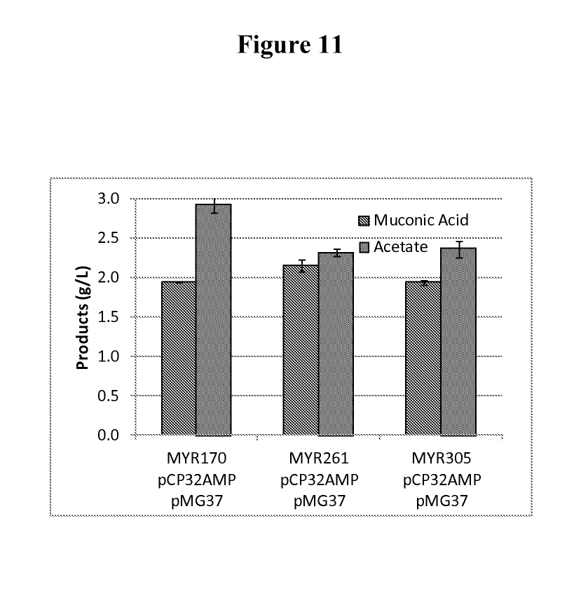

FIG. 11 provides the titer for muconic acid and acetic acid production in MYR170, MYR261 and MYR305 strains of E. coli after transformation with the plasmids pCP32AMP and pMG37. MYR305 is derived from MYR170 by means of deleting poxB gene from the chromosomal DNA while MYR261 is a MYR170 derivative wherein the poxB gene has been inactivated by means of inserting an additional copy of the tktA gene. As mentioned above, the plasmid pCP32AMP expresses the aroG gene coding for DAHP synthase protein functioning in the shikimic acid biosynthetic pathway leading to the accumulation of DHS due to the deletion of aroE gene in the E. coli strains MYR170, MYR261 and MYR305. With the expression of muconic pathway genes namely aroZ, aroY and catAX on the plasmid pMG37, the DHS is converted into cis,cis-muconic acid as illustrated in FIG. 2. With the presence of an additional copy of the aroB gene and the tktA gene in the MYR261 strain, there was a slight increase in the production of muconic acid accompanied by a decrease in the accumulation of acetic acid.

### Example 5

### Overexpression of talA or talB

The talB gene encodes the predominant transaldolase in E. coli, but the talA gene also encodes a minor transaldolase. Overproduction of transaldolase is known to improve flux into the aromatic pathway (Lu and Liao, 1997; Sprenger, 1995; Sprenger et al, 1995b). In the prior art, this was accomplished by overexpression of the tal gene (now known to be the talB gene) on a multicopy plasmid from its native promoter (Lu et al., 1997, Sprenger et al., 1995b). However, such plasmids are unstable, and require antibiotic selection for maintenance. Thus, there is a need for an improved process. Improved expression of talB can be obtained, for example, by substituting the native talB promoter in the chromosome with a strong constitutive promoter, for example a P₁₅ or P₂₆ promoter from Bacillus subtilis phage SPOT (SEQ ID No. 1 and SEQ ID No. 2, respectively), or the P_{R} promoter from bacteriophage lambda (SEQ ID No. 3). This is accomplished in two steps as described in Example 1, except that the cam^{R}, sacB cassette is used to replace the native chromosomal talB promoter in the first step. In the second step, the strong constitutive promoter is installed by transforming with a linear DNA comprising the strong constitutive promoter, flanked by at least 50 bases of the 5′ end of the talB coding region on the downstream side and at least 50 base pairs of homology just upstream of the native talB promoter on the upstream side of the strong constitutive promoter, and selecting for sucrose resistance. The talA gene can also be overexpressed by a similar method, but it is preferred to over express the talB gene, since it encodes the predominant activity (Sprenger, 1995; Sprenger et al, 1995b). See Example 4 for more details on construction of the expression cassette designed for overexpression.

### Example 6

### Expression of aroZ, aroY and catAX Genes

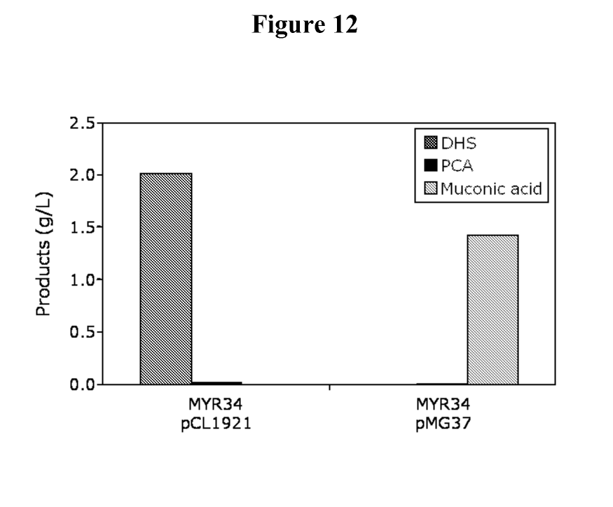

To demonstrate conversion of endogenous DHS produced by E. coli into muconic acid, heterologous genes catAX from Acinetobacter sp. ADP1, aroY from Klebsiella pneumoniae, and quiC from Acinetobacter sp. ADP1, were cloned under strong constitutive promoters (P₁₅, P_{R}, and P₂₆, respectively) in a low-copy plasmid, pCL1921 (Lerner and Inouye, 1990) to generate a ‘muconic plasmid’ pMG37. MYR34 strain derivatives carrying the empty vector (pCL1921) or pMG37 were grown at 37° C. for 17 hrs. in a shake flask medium (NBS minimal medium supplemented with aromatic amino acids and vitamins) containing 2% glucose. Supernatants were collected and analyzed by HPLC. In contrast to MYR34/pCL1921 which shows accumulation of DHS, MYR34/pMG37 shows production of muconic acid (FIG. 12). No significant amount of DHS, or intermediate products such as PCA and catechol were detected from the latter strain, suggesting that the heterologous genes expressed from pMG37 were functional and sufficient.

### Example 7

### Comparison of aroZ Homologs

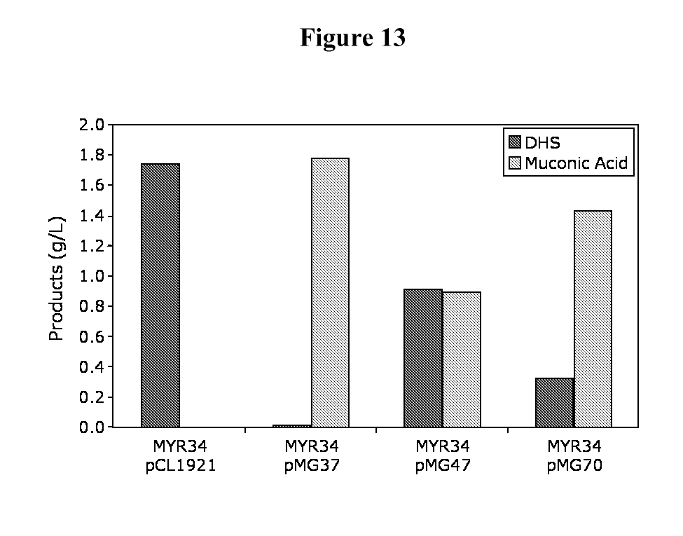

Three different aroZ homologs and analogs were compared (FIG. 13) for their ability to divert DHS into the muconic acid production pathway. quiC from Acinetobacter sp. ADP1, asbF from Bacillus thuringiensis, and qa-4 from Neurospora crassa, are reported to encode for proteins that have AroZ-like activity (Elsemore and Ornston, 1995; Fox et al, 1995; Rutledge, 1984). Each of these genes was codon-optimized for expression in E. coli and synthesized by GeneArt (Invitrogen), and cloned under a strong constitutive P₂₆ promoter in low-copy ‘muconic plasmid’ which also expressed catAX and aroY genes from the P₁₅ and P_{R} promoters, respectively. MYR34/pCL1921, MYR34/pMG37 (muconic plasmid with quiC as aroZ), MYR34/pMG47 (muconic plasmid with asbF as aroZ), and MYR34/pMG70 (muconic plasmid with asbF as aroZ) were grown at 37° C. for 48 hrs. in a shake flasks with minimal medium containing 2% glucose, the aromatic amino acids and aromatic vitamins. Supernatants were collected and analyzed by HPLC. As expected, empty vector transformed MYR34 accumulated DHS and produced no muconic acid. The two aroZ homologs and the one analog examined were functional in diverting DHS towards muconic acid production, but to a varying degree. The MYR34 derivative expressing quiC gene was most robust and showed nearly 100% conversion of DHS to muconic acid with insignificant amount of DHS retention. The MYR34 derivative expressing the fungal aroZ homologue, qa-4, followed close with about 80% conversion of DHS to muconic acid and 20% DHS retention. Lastly, the MYR34 derivative expressing asbF gene showed only 50% conversion of DHS to muconic acid and 50% DHS retention. Taken together, under our shake flask assay conditions, the expression and/or activity of quiC gene appeared to be the highest compared to that of other aroZ homologs.

### Example 8

### Chromosomal Integration of catAX, aroY and quiC

Muconic acid can be produced by strains that contain only chromosomally integrated single copies of catA-X, aroY and quiC expressed from constitutive promoters at adhE locus.

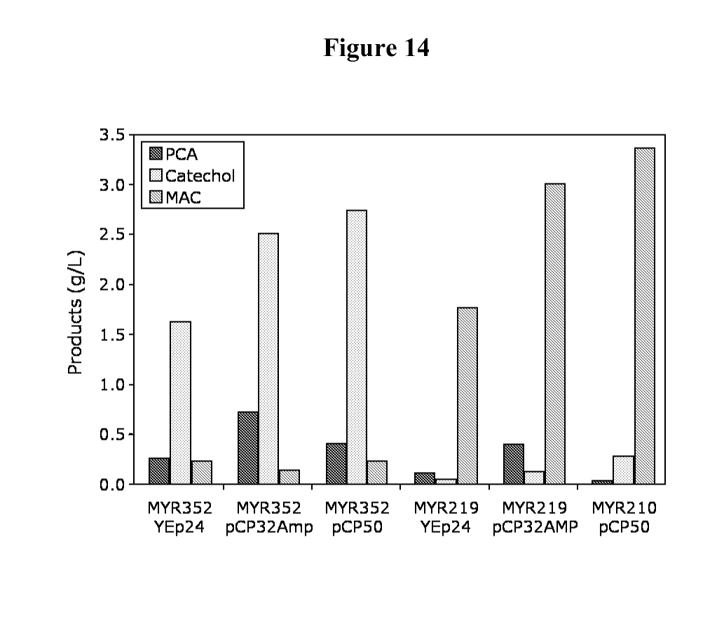

MYR170 (ΔaroE, Δack::P₁₅-aroB), a high DHS producer, was the host strain used for integrating the muconic acid pathway genes at the adhE locus in the chromosome (SEQ ID No. 41). The resulting strain MYR352 was transformed with plasmids YEp24 (medium-copy, empty vector), pCP32AMP (medium-copy, aroG expressed from native promoter), or pCP50 (medium-copy, aroG and tktA expressed from their respective native promoters) to generate derivative strains. The latter two plasmids were used to increase DHS production. Strains were grown at 37° C. for 72 hrs. in shake flask medium containing 2% glucose as described above. Supernatants were collected at 72 hrs. and analyzed by HPLC. As expected, the aroG and aroG/tktA transformed MYR352 derivatives showed an overall increase in total product formation compared to an empty vector control (FIG. 14). All of the MYR352 transformants produced measurable titers of muconic acid, demonstrating for the first time that muconic acid can be produced by a strain that contains only integrated “muconic pathway” genes and without a fed chemical inducer of gene expression.

Not all DHS that was produced in any of these MYR352 derivative strains was converted to the end product muconic acid. Instead, there was a significant amount of catechol accumulation (FIG. 14), suggesting that expression or activity of catAX is limiting when it is expressed from a single copy on chromosome. Since the major accumulating intermediate was catechol, it is likely that quiC and aroY gene expression and/or activity is sufficient in the MYR352 strain background for muconic acid synthesis.

The MYR352 strain derivatives were compared in parallel with analogous MYR219 strain derivatives. MYR219 strain is same as MYR170 strain but contains low-copy plasmid pMG37 expressing muconic acid pathway genes. Thus, the main difference between MYR352 and MYR219 strains is with reference to the dosage of muconic acid pathway genes (1 copy vs. about 5 copies, respectively). In contrast to MYR352 derivative strains, MYR219 derivative strains showed very little accumulation of catechol or other intermediates, and successfully produced the end product muconic acid. Together, these results indicate the need for increasing catAX activity in strains such as MYR352.

### Example 9

### Expression of catAX

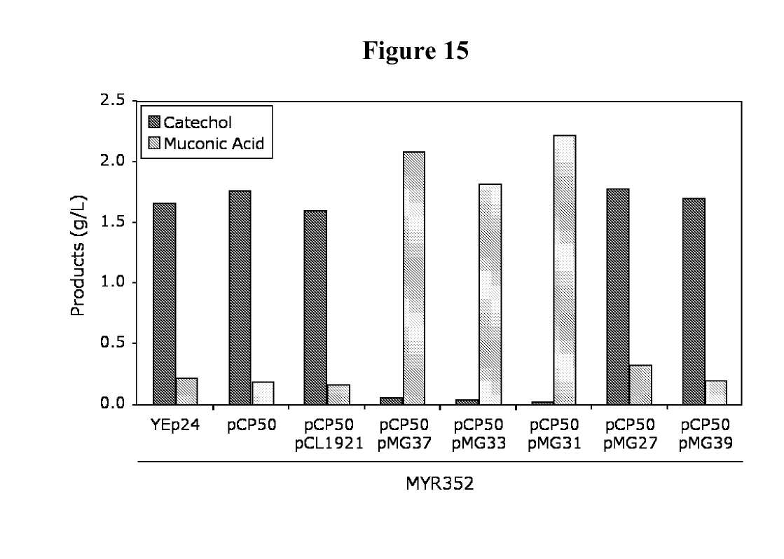

Accumulation of catechol and inefficient production of muconic acid in MYR352 strain is due to limiting dosage and/or activity of the catAX gene product(s). As described above, MYR352 contains ΔaroE, Δack::P₁₅-aroB and chromosomally integrated single copies of catAX, aroY and quiC genes under strong constitutive promoters. This strain was transformed with medium-copy empty vector control (YEp24) or aroG/tktA expression plasmid (pCP50) to increase carbon flow into the aromatic amino acid synthesis pathway and produce high amounts of DHS. Growth of transformed strains at 37° C. for 72 hrs in shake flask medium supplemented with 2% glucose as described above resulted in accumulation of catechol intermediate. This result suggested that catAX activity may be insufficient in MYR352. To confirm this hypothesis, the ability of one or more muconic acid pathway genes expressed from low-copy plasmid to alleviate catechol accumulation in MYR352/pCP50 was tested (FIG. 15). Specifically, MYR352/pCP50 was further transformed with low-copy empty vector control (pCL1921) or plasmids expressing all three genes, two genes, or one gene of the muconic acid production pathway. The derivative strains were assayed in a shake flask experiment as described above. While increasing the dosage of aroY alone (from pMG27) or quiC alone (from pMG39) did not alleviate catechol accumulation, expression of all of the muconic acid pathway genes (from pMG37) or catAX and aroY together (from pMG33), resulted in successful conversion of catechol to muconic acid. Further, expression of catAX alone (from pMG31) was sufficient for production of muconic acid and preventing accumulation of catechol.

### Example 10

### Constructing a Leaky aroE Mutation

In the prior art process for producing cis,cis-muconic acid, the host strain contains a mutation in the aroE gene named aroE353, which is a null mutation. As a result, the strain requires the feeding of the aromatic amino acids (phenylalanine, tyrosine, and tryptophan) and aromatic vitamins made from the shikimate pathway (p-hydroxy benzoic acid, p-amino benzoic acid, and 2,3-dihydroxy benzoic acid). The aromatic amino acids are too expensive to be fed in a commercially attractive process. As such, the prior art process needs a substantial improvement. This can be accomplished by installing a leaky version of the aroE gene, that we shall call aroE*. Leaky mutations are obtained by first generating a missense mutation that changes one amino acid in the aroE coding sequence that results in a null phenotype. This can be accomplished by any form of mutagenesis and screening for simultaneous auxotrophy for the six aromatic compounds listed above. A preferred method is to create a pool of mutant aroE genes by error-prone PCR mutagenesis, using Taq DNA polymerase, using wild type E. coli C genomic DNA as the template, and using PCR oligonucleotide primers that hybridize about 1000 base pairs upstream and 1000 base pairs downstream of the aroE coding region. The resulting pool of linear DNA molecules is used to transform an E. coli C derivative that produces cis,cis-muconic acid, and which contains an integrated cam^{R}, sacB cassette that has replaced the aroE coding region (see Example 4 for a related example), and selecting for sucrose resistance. The transformants are then screened for auxotrophs that have lost chloramphenicol resistance and require the six aromatic compounds listed above. Several independent auxotrophs are picked and tested for revertability by plating about 10⁷, 10⁸, or 10⁹ cells (rinsed in minimal glucose medium) on a minimal glucose plate without the six aromatic compounds. Revertants that give rise to colonies on the plates are picked and tested for production of cis,cis-muconic acid, but without production of substantial levels of aromatic amino acids. Among such revertants will be strains that carry one or more mutations in the aroE gene, such that the AroE enzyme provides enough aromatic amino acids and vitamins for growth, but not a surplus of these aromatic compounds. Another method to obtain a leaky aroE mutant is to install one of the classical revertable aroE mutants, such as aroE353 and aroE24 (both available from the Coli Genetic Stock Center at Yale University, New Haven, Conn., USA), into a cis,cis-muconic acid producing strain, and select for revertants as described above.

### Example 11

### Import of Glucose by Facilitated Diffusion

One of the substrates in the first committed step of the aromatic pathway is phosphoenolpyruvate (PEP). PEP is also the source of phosphate and energy for importing glucose and some other sugars by the bacterial phosphotransferase system (PTS). Thus, when a bacterium is growing on a PTS-dependent sugar, there is competition between the PTS and the aromatic pathway for PEP. As such, a significant improvement in increasing flux to the aromatic pathway can be achieved by deleting the PTS and providing an alternative pathway for sugar uptake. One solution to this problem is to replace the PTS with the E. coli GalP permease, a proton symporter that works reasonably well for glucose uptake (U.S. Pat. No. 6,692,794). However, the proton symporter still uses energy to maintain the proton gradient that is necessary to drive the permease. As such, there is a need for even further improvement in the process.

Some sugars, such as xylose, can be imported by a transporter protein that derives energy from hydrolysis of ATP (adenosine triphosphate). Once again, if the energy-dependent transporter can be replaced by a transporter that requires less energy, then an improvement can be made, since the energy inherent in the ATP can be conserved for other beneficial uses.

A significant improvement can be obtained by using a facilitated diffusion transporter, which expends no energy for the importation of the sugar (Parker et al, 1995; Snoep et al, 1994). For example, the glucose facilitator from Zymomonas mobilis, encoded by the glf gene, can be used in place of, or in addition to, the PTS in 3-dehydroshikimate (DHS) producing strains (Yi et al., 2003). However, these strains still rely at least partly on GalP for glucose import. Since GalP requires energy in the form of a proton gradient for importation of glucose, there is a need for improvements in the efficiency of glucose import for muconic producing strains.

A cassette for expression of glf plus a glucokinase gene, glk, also from Z. mobilis, can be assembled with a strong constitutive promoter, for example P₂₆. This cassette can then be integrated into the genome of a host strain at a location that will not interfere with production of the desired compound, which in this case is cis,cis-muconic acid. An example of such a location in the E. coli chromosome is the threonine degradation operon, tdcABCDEFG. If the growth medium contains no threonine, then this operon is not needed or expressed, so an insertion of an expression cassette in that operon does not interfere with metabolism.

To achieve the above described improvement, one or more of the genes encoding a PTS function are deleted, using a method similar to that disclosed in Example 1. For example, one or more of ptsH, ptsI, crr, or ptsG can be deleted. Next galP is deleted. The P₂₆-glf, glk cassette can then be installed in two steps, similar to those described in Example 1. In the first step, a cam^{R}, sacB cassette is integrated at the tdc operon, using a linear DNA derived from pAC21 (SEQ ID No. 15), and selecting for chloramphenicol (30 mg/l) resistance. In the second step, the P₂₆-glf, glk cassette is integrated at the tdc operon, using a linear DNA derived from pAC19 (SEQ ID No. 15), selecting for sucrose resistance and screening for chloramphenicol sensitivity, and in this case, improved growth on minimal glucose medium.

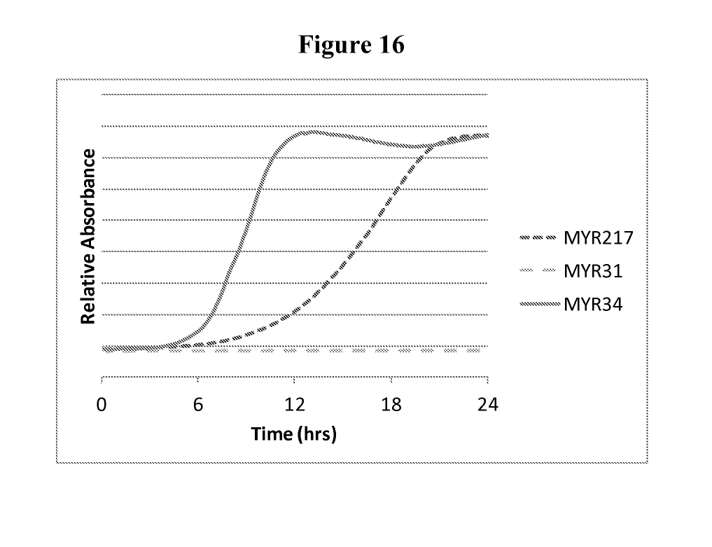

To test whether facilitated diffusion of glucose could substitute for the conventional glucose import systems in E. coli, the ptsHI genes and the galP gene were deleted from MYR34 (ΔaroE), and then the P₂₆-0; glk cassette was integrated at the tdc operon, using a linear DNA derived from pAC19 (SEQ ID No. 14), to give strain MYR217. MYR217 grows reasonably well on a minimal glucose medium supplemented with the required three aromatic amino acids and three aromatic vitamin-like compounds (FIG. 16). However, strain MYR31, which contains deletions of ptsHI and galP, but does not contain the glf, glk cassette did not show any measurable growth (FIG. 16). Thus, facilitated diffusion is sufficient to replace the two conventional glucose import systems in our strain background.

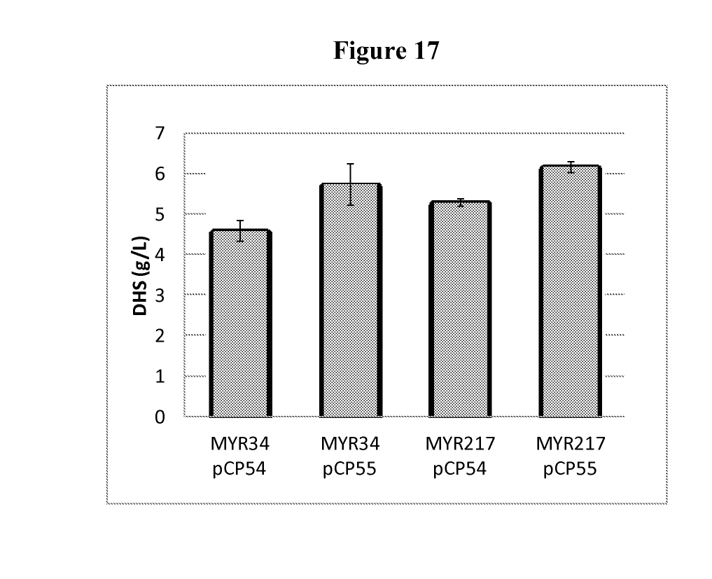

To test whether facilitated diffusion is useful for producing compounds derived from the aromatic pathway, MYR34 and MYR217 were transformed with pCP54 (aroG, aroB) and pCP55 (aroG, aroB, tktA). Production of the aromatic intermediate 3-dehydroshikimate (DHS) in shake flasks was compared for these two strains (FIG. 17). With either pCP54 or pCP55, the strain using facilitated diffusion produced as much or more DHS than the strains using the conventional glucose import systems. Production of DHS is a good proxy for muconic acid production in engineered E. coli strains, so we can conclude that facilitated diffusion of glucose is a useful improvement for muconic acid production.

### Example 12

### Overexpression of the aroB Gene

Expression of the aroB gene is reported to be rate limiting for cis,cis-muconic acid production (Niu et al., 2002). In the prior art, this was allegedly solved by integrating a second copy of the aroB gene with its native promoter. However, this is insufficient to alleviate the aroB limitation, since the native promoter and ribosome binding site of the aroB gene are far from ideal. As such, the process needs substantial improvement.

Improved overexpression of aroB can be obtained, for example, by replacing the native aroB promoter in the chromosome with a strong constitutive promoter, for example a P₁₅ or P₂₆ promoter from Bacillus subtilis phage SPOT (SEQ ID No. 1 and SEQ ID No. 2, respectively), or the P_{R} promoter from bacteriophage lambda (SEQ ID No. 3). This is accomplished in two steps as described in Example 4, except that the cam^{R}, sacB cassette is used to replace the native chromosomal aroB promoter and/or ribosome binding site in the first step. In the second step, the strong constitutive promoter is installed by transforming with a linear DNA comprising the strong constitutive promoter, followed by a ribosome binding site and at least 50 bases from the 5′ end of the aroB coding sequence, including the ATG start codon, on the downstream side, and at least 50 base pairs of homology just upstream of the native aroB promoter on the upstream side of the strong constitutive promoter, and selecting for sucrose resistance. In addition to, or instead of, installing a stronger promoter, using a similar method, a stronger ribosome binding site, for example, AGGAGG, can be installed about 4 to 10 base pairs upstream of the ATG translation start codon of aroB. A copy of such a synthetic cassette, for example, a P₁₅-aroB cassette, can be integrated in the chromosome at a locus distinct from the native aroB locus, for example at the ack locus. Simultaneous deletion of the ack gene, as well as deleting the poxB gene as in Example 4 can help to reduce formation of unwanted acetate during fermentations.

### Example 13

### Decreasing Flux Through the Oxidative Branch of the Pentose Phosphate Pathway

The erythrose-4-phosphate that is needed for the first committed step in the aromatic pathway is derived from the non-oxidative portion of the pentose phosphate pathway (PPP). There are two different pathways by which carbon can enter the PPP. The first is from glucose-6-phosphate by the enzymes glucose-6-phosphate dehydrogenase (encoded by the zwf gene), 6-phosphogluconolactonase (encoded by the pgl gene), and 6-phophogluconate dehydrogenase (encoded by the gnd gene), to give ribulose-5-phosphate. In the last of these three steps, one carbon is lost as CO₂. This path into the PPP is called the oxidative branch of the PPP. Ribulose-5-phosphate is then converted into a variety of other sugar phosphates by the action of isomerases, epimerases, transketolase, and transaldolase. This group of reversible reactions, starting with ribulose-5-phosphate, is called the non-oxidative branch of the PPP. The second path by which carbon can enter the PPP is through fructose-6-phosphate and glyceraldehye-3-phosphate (both of which come from the Embden-Myerhoff pathway, also known as glycolysis), which are combined and rearranged by transaldolase and transketolase to give the variety of other sugar phosphates, one of which is erythrose-4-phosphate. If carbon enters the PPP through this second route, then no CO₂ is lost. In order to improve the yield of cis,cis-muconic acid from glucose, the loss of CO₂ can be prevented by blocking the oxidative branch of the PPP, such that all carbon entering the PPP must come through a non-oxidative route from fructose-6-phosphate and glyceraldehye-3-phosphate. The blocking of the oxidative branch of the PPP is accomplished by deleting the zwf gene, using a two step method similar to that disclosed in Example 1 for deleting the tyrR gene.

### Example 14

### Increasing the Flux to and Through PEP to the Aromatic Pathway

It is desirable to ensure that PEP is not a rate limiting intermediate on the pathway to cis,cis-muconic acid. This is accomplished, for example, by increasing the recycling of pyruvate to PEP by the enzyme PEP synthetase, which is accomplished by integrating an overexpression cassette of the pps gene as described above in other examples. Another approach is to limit the consumption of PEP by pyruvate kinase, which in E. coli is encoded by the pykA and pykF genes. In this case, the approach is to decrease the activity of the enzyme(s). This is accomplished by deleting one or more genes that encode pyruvate kinase (as described in Example 1 for tyrR), or reducing the strength of expression of one or more of these genes, for example, by mutating the promoter, ribosome binding site, or coding sequence, such that the level of pyruvate kinase activity is decreased. For example, the RBS in front of the E. coli pykA gene is 5′CGGAGTATTACATG. The ATG translation start codon is underlined. This sequence can be mutated to CaGAGTATTACATG, CaaAGTATTACATG, CaatGTATTACATG, CaataTATTACATG, and so on, such that the RBS sequence is made less like the consensus RBS of AGGAGG by one base change at a time. Each mutated version is then introduced into the chromosome at the pykA locus, replacing the wild type, and cis,cis-muconic acid production levels are measured for improvement.

### Example 15

### Conferring Growth on Sucrose

Strains derived from E. coli C do not grow on sucrose as a sole carbon source. However, they can be genetically engineered to do so as disclosed in PCT Patent Application PCT/US11/064,598 which is hereby incorporated by reference in its entirety. As such, a cis,cis-muconic acid producing strain can be engineered to grow on sucrose as disclosed in the above mentioned application.

### Example 16

### An Improved Producer of cis,cis-muconic Acid

All of the features described in Examples 1-15 can be combined in one strain of E. coli by installing the features one after another. The resulting strain comprises an improved cis,cis-muconic acid producer. The resulting strain can then be even further improved by integrating a second copy of each overexpression cassette described above, one at a time, at a location separate from the location of the first copy. An example of a convenient and safe location is at a BsrB1 restriction site just downstream from the terminator of rrfF, which encodes a ribosomal RNA. The desired cassette is ligated as a blunt linear DNA into the unique BsrB1 site of plasmid pMH17F (SEQ ID No. 17). An example is the ligation of the catAX expression cassette to give a plasmid named pcatAX. In parallel, a cam^{R}, sacB cassette is ligated as a blunt fragment into pMH17F to give pMH28F (SEQ ID No. 19). A linear DNA derived from pMH28 by PCR or by restriction enzyme cutting is used to deposit the cam^{R}, sacB cassette at the rrfF site. Next, a linear DNA derived from pcatAX by PCR or by restriction enzyme cutting is used to install the second copy of the catAX cassette at the rrfF locus, using selection on sucrose. The resulting strain is then compared with its grandparent strain for cis,cis-muconic acid production to determine that catAX was a limiting step. By a similar method, each cassette from Examples 2-15 is tested for a rate limiting step. If a step is found to be rate limiting, then one or more additional copies of the relevant cassette is/are integrated at yet other appropriate locations in the chromosome, leading to still further improvements in cis,cis-muconic acid production, without the need for plasmids or inducible promoters.

### Example 17

### Production of cis,cis-muconic Acid by Fermentation

Cis, cis-muconic acid can be produced by genetically engineered microorganisms disclosed in the above Examples 1-15. The growth medium can vary widely and can be any medium that supports adequate growth of the microorganism. A preferred medium is a minimal medium containing mineral salts and a non-aromatic carbon source, such as glucose, xylose, lactose, glycerol, acetate, arabinose, galactose, mannose, maltose, or sucrose (see above for an example of a preferred minimal growth medium). For each combination of engineered microorganism and growth medium, appropriate conditions for producing cis,cis-muconic acid are determined by routine experiments in which fermentation parameters are systematically varied, such as temperature, pH, aeration rate, and compound or compounds used to maintain pH. As cis,cis-muconic acid is produced, one or more compounds must be fed into the fermentor to prevent pH from going too low. Preferred compounds for neutralizing the acid include alkaline salts such as oxides, hydroxides, carbonates, and bicarbonates of ammonium, sodium, potassium, calcium, magnesium, or a combination two or more of such alkaline salts.

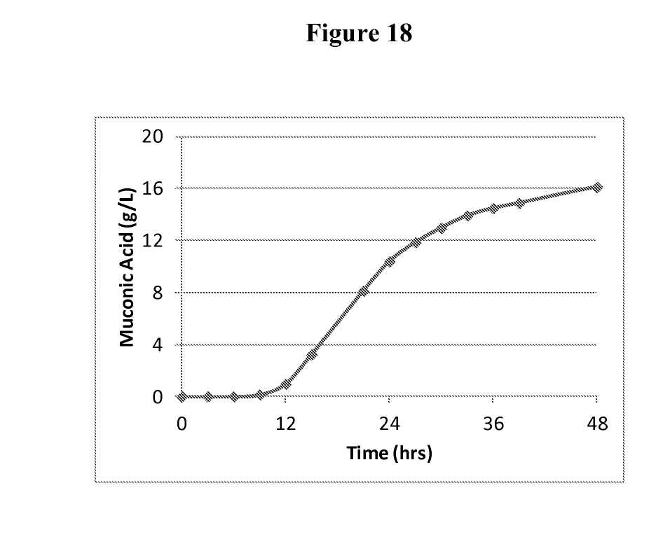

Muconic acid production by MYR428 strain of E. coli in a 7 Liter fermentor is shown in FIG. 18. MYR261 strain of E. coli with a genotype of ΔaroE ΔackA::P₁₅-aroB ΔpoxB::tktA was transformed with the plasmids pCP32AMP and pMG37 to generate MYR428. MYR428 was grown in a 7 liter fermentor as described above with glucose feeding for 48 hours. The final muconic acid titer was 16 g/l (see FIG. 18).

After fermentation is complete, cells are removed by flocculation, centrifugation, and/or filtration, and the cis,cis-muconic acid is then purified from the clarified broth by a combination of one or more subsequent steps, for example precipitation, crystallization, electrodialysis, chromatography (ion exchange, hydrophobic affinity, and/or size based), microfiltration, nanofiltration, reverse osmosis, and evaporation.

TABLE 1
Bacterial strains and plasmids used in the present invention

| | |
|---|---|
| Bacterial strain/ | |
| Plasmid | Characteristics |

Bacterial Strains

| | |
|---|---|
| ATCC8739 | Escherichia coli “C” wild type |
| MYR34 | ATCC8739 ΔaroE |
| MYR170 | ATCC8739 ΔaroE, ΔackA::P₁₅aroB |
| MYR261 | ATCC8739 ΔaroE, ΔackA::P₁₅aroB, ΔpoxB::tktA |
| MYR305 | ATCC8739 ΔaroE, ΔackA::P₁₅aroB, ΔpoxB |
| MYR31 | ATCC8739 ΔptsHI, ΔgalP |
| MYR217 | ATCC8739 ΔptsHI, ΔgalP, Δtdc::glf-glk, ΔaroE |
| MYR352 | ATCC8739 ΔaroE, ΔackA::P₁₅aroB, ΔadhE::P₁₅-catAX, P_{R}-aroY, P₂₆- |
| | quiC |
| RY903 | ΔaroE, ΔackA::P₁₅aroB, pMG37, aroG*20-893 |
| RY909 | ΔaroE, ΔackA::P₁₅aroB, pMG37, aroG*20-899 |
| RY911 | ΔaroE, ΔackA::P₁₅aroB, pMG37, aroG*20-901 |
| RY912 | ΔaroE, ΔackA::P₁₅aroB, pMG37, aroGwt |
| RY913 | ΔaroE, ΔackA::P₁₅aroB, pMG37, aroG*20-893, ΔtyrR::kan |
| RY919 | ΔaroE, ΔackA::P₁₅aroB, pMG37, aroG*20-899, ΔtyrR::kan |
| RY921 | ΔaroE, ΔackA::P₁₅aroB, pMG37, aroG*20-901, ΔtyrR::kan |
| RY922 | ΔaroE, ΔackA::P₁₅aroB, pMG37, aroGwt, ΔtyrR::kan |

Plasmids

| | |
|---|---|
| YEp24 | 2μ yeast origin, URA3, Tc^{R}, pMB1 replicon, Ap^{R} |
| pCP32AMP | 2μ yeast origin, URA3, Tc^{R}, pMB1 replicon, Ap^{R}, aroG |
| pCP14 | 2μ yeast origin, URA3, Tc^{R}, pMB1 replicon, Ap^{R}, P₁₅aroB |
| pCP54 | 2μ yeast origin, URA3, Tc^{R}, pMB1 replicon, Ap^{R}, P₁₅aroB, aroG |
| pCP50 | 2μ yeast origin, URA3, Tc^{R}, pMB1 replicon, Ap^{R}, aroG, tktA |
| pCP55 | 2μ yeast origin, URA3, Tc^{R}, pMB1 replicon, Ap^{R}, aroG, aroB, tktA |
| pCL1921 | pSC101 replicon, Spc^{R} |
| pMG27 | pSC101 replicon, Spc^{R}, P_{R}-aroY |
| pMG31 | pSC101 replicon, Spc^{R}, P₁₅-catAX |
| pMG33 | pSC101 replicon, Spc^{R}, P₁₅-catAX, P_{R}-aroY |
| pMG37 | pSC101 replicon, Spc^{R}, P₁₅-catA-CatX, P_{R}-aroY, P₂₆-quiC |
| pMG39 | pSC101 replicon, Spc^{R}, P₂₆-quiC |
| pMG47 | pSC101 replicon, Spc^{R}, P₁₅-catAX, P_{R}-aroY, P₂₆-asbF |
| pMG70 | pSC101 replicon, Spc^{R}, P₁₅-catAX, P_{R}-aroY, P₂₆-qa-4 |

TABLE 2
Sequence Information

| No. | Name | Description |
|---|---|---|
| 1 | SEQ ID No. 1 | The P₁₅ promoter from Bacillus subtilis phage SP01, with a stem and loop added just |
| | | downstream from the transcription start site. |
| 2 | SEQ ID No. 2 | The P₂₆ promoter from Bacillus subtilis phage SP01 |
| 3 | SEQ ID No. 3 | The P_{R} promoter from Escherichia coli phage |
| 4 | SEQ ID No. 4 | Protein sequence of 3-dehydroshikimate dehydratase from Neurospora crassa encoded |
| | | by the qa-4 gene. |
| 5 | SEQ ID No. 5 | Genomic DNA sequence of the qa-4 gene from Neurospora crassa plus surrounding |
| | | sequences. |
| 6 | SEQ ID No. 6 | Protein sequence of 3-dehydroshikimate dehydratase from Aspergillus nidulans. |
| | | encoded by the qutC gene |
| 7 | SEQ ID No. 7 | Genomic DNA sequence of the qutC gene from Aspergillus nidulans plus surrounding |
| | | sequences |
| 8 | SEQ ID No. 8 | Protein sequence of protocatechuate decarboxylase (AroY) from Klebsiella pnemoniae |
| | | ATCC25597 |
| 9 | SEQ ID No. 9 | DNA sequence of the aroY gene of Klebsiella pneumoniae 342 plus 2 kilobases of |
| | | surrounding DNA sequences |
| 10 | SEQ ID No. 10 | DNA sequence of the catA gene from Acinetobacter baylyi ADP1, including 410 bases of |
| | | upstream sequence and two open reading frames downstream |
| 11 | SEQ ID No. 11 | Protein sequence of CatA (catechol 1,2-dioxygenase) from Acinetobacter baylyi ADP1 |
| 12 | SEQ ID No. 12 | DNA sequence of the quiC (3-dehydroshikimate dehydratase) gene from Acinetobacter |
| | | sp. ADP1 |
| 13 | SEQ ID No. 13 | Codon-optimized DNA sequence of the quiC (3-dehydroshikimate dehydratase) gene |
| | | from Acinetobacter sp. ADP1 |
| 14 | SEQ ID No. 14 | Protein sequence of QuiC (3-dehydroshikimate dehydrogenase from Acinetobacter sp. |
| | | ADP1 |
| 15 | SEQ ID No. 15 | DNA sequence of the plasmid pAC21 |
| 16 | SEQ ID No. 16 | DNA sequence of the plasmid pAC19 |
| 17 | SEQ ID No. 17 | DNA sequence of the plasmid pMH17F |
| 18 | SEQ ID No. 18 | DNA sequence of the coding region of the wild type aroG gene |
| 19 | SEQ ID No. 19 | DNA sequence of the plasmid pMH28F |

TABLE 3
Sequence Information - cont.

| No. | Name | Description |
|---|---|---|
| 20 | SEQ ID No. 20 | DNA sequence of the plasmid pCL1921 |
| 21 | SEQ ID No. 21 | DNA sequence of the plasmid pMG27 |
| 22 | SEQ ID No. 22 | DNA sequence of the plasmid pMG31 |
| 23 | SEQ ID No. 23 | DNA sequence of the plasmid pMG33 |
| 24 | SEQ ID No. 24 | DNA sequence of the plasmid pMG37 |
| 25 | SEQ ID No. 25 | DNA sequence of the plasmid pMG39 |
| 26 | SEQ ID No. 26 | DNA sequence of the plasmid pMG47 |
| 27 | SEQ ID No. 27 | DNA sequence of the plasmid pMG70 |
| 28 | SEQ ID No. 28 | DNA sequence of the plasmid pCP32AMP |
| 29 | SEQ ID No. 29 | DNA sequence of the plasmid pCP14 |
| 30 | SEQ ID No. 30 | DNA sequence of the plasmid pCP50 |
| 31 | SEQ ID No. 31 | DNA sequence of the plasmid pCP54 |
| 32 | SEQ ID No. 32 | DNA sequence of the plasmid pCP55 |
| 33 | SEQ ID No. 33 | DNA sequence of the plasmid YEP24 |
| 34 | SEQ ID No. 34 | DNA sequence of the deleted aroE region |
| 35 | SEQ ID No. 35 | DNA sequence of the integrated cassette |
| | | Δack::P₁₅aroB |
| 36 | SEQ ID No. 36 | DNA sequence of the ΔpoxB region |
| 37 | SEQ ID No. 37 | DNA sequence of the integrated cassette |
| | | ΔpoxB::tktA |
| 8 | SEQ ID No. 38 | DNA sequence of the ΔptsHI region |
| 39 | SEQ ID No. 9 | DNA sequence of the integrated cassette |
| | | Δtdc::glf-glk |
| 40 | SEQ ID No. 40 | DNA sequence of the ΔgalP region |
| 41 | SEQ ID No. 41 | MYR352 ΔadhE::P₁₅-catAX, P_{R}-aroY, P₂₆-quiC |

TABLE 4
aroG*mutant alleles that lead to resistance
to phenylalanine feedback inhibition

| Strain | Allele number | Nucleotide mutation | Amino acid mutation |
|---|---|---|---|
| RY893 | aroG*20-893 | C449T | Pro150Leu |
| RY897 | aroG*20-897 | C449T | Pro150Leu |
| RY899 | aroG*20-899 | T538C | Ser180Pro |
| RY901 | aroG*20-901 | C438T | Pro150Ser |
| MYR450 | aroG*111 | C55T | Pro19Ser |
| MYR451 | aroG*211 | G533A | Gly178Glu |
| MYR452 | aroG*212 | C540T | Ser180Phe |
| MYR453 | aroG*311 | Deletion from base | Deletion of |
| | | pair 36 to 44bp | Glu-Ile-Lys |
| MYR454 | aroG*312 | C632T | Ser211Phe |
| MYR455 | aroG*411 | T29A | Ile10Asn |
| MYR456 | aroG*412 | G533A | Gly178Glu |
| MYR457 | aroG*511 | C448T | Pro150Ser |

TABLE 5
AroG activity measurement in crude extract
from various recombinant E. coli strains

| | | Specific activity mU | |
|---|---|---|---|
| | | (One mU = one nM product | % of activity |
| | | made per milligram protein | resistant to |
| Strain | aroG* allele | per minute) | phenylalanine |
| RY893 | aroG* 20-893 | 62 | 34 |
| RY897 | aroG* 20-897 | 55 | 77 |
| RY899 | aroG* 20-899 | 92 | 113 |
| RY901 | aroG* 20-901 | 78 | 76 |
| RY902 | aroG wild type | 38 | 6 |
| RY890 | aroG wild type | 54 | 7 |

TABLE 6
Muconic acid production in shake flasks by strains
containing feedback resistant aroG* alleles

| Strain | Muconic acid titer g/l |
|---|---|
| RY913 | 3.04 |
| RY919 | 3.11 |
| RY921 | 2.99 |
| RY922 | 1.45 |

### REFERENCES

U.S. Pat. No. 4,480,034
U.S. Pat. No. 4,535,059
U.S. Pat. No. 4,588,688
U.S. Pat. No. 4,608,338
U.S. Pat. No. 4,681,852
U.S. Pat. No. 4,753,883
U.S. Pat. No. 4,833,078
U.S. Pat. No. 4,968,612
U.S. Pat. No. 5,168,056
U.S. Pat. No. 5,272,073
U.S. Pat. No. 5,487,987
U.S. Pat. No. 5,616,496
U.S. Pat. No. 6,600,077
U.S. Pat. No. 6,180,373
U.S. Pat. No. 6,210,937
U.S. Pat. No. 6,472,169
U.S. Pat. No. 6,613,552
U.S. Pat. No. 6,962,794
U.S. Pat. No. 7,244,593
U.S. Pat. No. 7,638,312
U.S. Pat. No. 7,790,431
US Patent Application Publication No. US 2009/0191610
U.S. Patent Application Publication No. US 2010/0314243 A1
European Patent Application No. 86300748.0
International Patent Application Publication No. WO 2011/017560
International Patent Application Publication No. WO 2011/085311
International Patent Application Publication No. WO 2011/123154
Altschul, S. F., Gish, W., Miller, W., Myers, E. W., and Lipman, D. J. (1990) Basic local alignment search tool, J Mol Biol 215, 403-410.
Altschul, S. F., Madden, T. L., Schaffer, A. A., Zhang, J., Zhang, Z., Miller, W., and Lipman, D. J. (1997) Gapped BLAST and PSI-BLAST: a new generation of protein database search programs, Nucleic Acids Res 25, 3389-3402.
Barbe, V., Vallenet, D., Fonknechten, N., Kreimeyer, A., Oztas, S., Labarre, L., Cruveiller, S., Robert, C., Duprat, S., Wincker, P., Ornston, L. N., Weissenbach, J., Marliere, P., Cohen, G. N., and Medigue, C. (2004) Unique features revealed by the genome sequence of Acinetobacter sp. ADP1, a versatile and naturally transformation competent bacterium, Nucleic Acids Res 32, 5766-5779.
Bird, J. A. and Cain, R. B. (1968) cis-cis-muconate, the product inducer of catechol 1,2-oxygenase in Pseudomonas aeruginosa. Biochem. J. 109, 479-481.
Bongaerts, J., Kramer, M., Muller, U., Raven, L. and Wubbolts, M. (2001) Metabollic engineering for microbial producitnof aromatic acids and derived compounds. Met. Eng. 3, 289-300.
Chandran, S. S., Yi, J., Draths, K. M., von Daeniken, R., Weber, W. and Frost, J. W. (2003) Phosphoenolpyruvate availability and the biosynthesis of shikimic acid. Biotechnol. Prog. 19, 808-814.
Chen, R., Hatzimanikatis, V., Yap, W. M. G. J., Potma, P. W. and Bailey, J. E. (1997) Metabolic consequences of phosphotransferase (PTS) mutation in a phenylalanie-producing recombinant Escherichia coli. Biotechnol. Prog. 13, 768-775.
Chen, K., Dou, J., Tang, S., Yang, Y., Wang, H., Fang, H. and Zhou, C. (2012) Deletion of the aroK gene is essential for high shikimic acid accumulation through the shikimate in E. coli. Bioresource Technol, 119, 141-147.
Choi, W. J., Lee, E. Y., Cho, M. H., and Choi, C. Y. (1997) Enhanced production of cis,cis-muconate in a cell-recycle bioreactor. J. Fermentation and Bioengineering. 84, 70-76.
Curran, K. A., Leavitt, J. M., Karim, A. S, and Alper, H. S. (2012) Metabolic engineering of muconic acid production in Saccharomyces cerevisiae. Metabol. Engineer. 15, 55-66.
de Berardinis, V., Vallenet, D., Castelli, V., Besnard, M., Pinet, A., Cruaud, C., Samair, S., Lechaplais, C., Gyapay, G., Richez, C., Durot, M., Kreimeyer, A., Le Fevre, F., Schachter, V., Pezo, V., Doring, V., Scarpelli, C., Medigue, C., Cohen, G. N., Marliere, P., Salanoubat, M., and Weissenbach, J. (2008) A complete collection of single-gene deletion mutants of Acinetobacter baylyi ADP1, Mol Syst Biol 4, 174.
Draths, K. M., Pompliano, D. L., Conley, D. L., Frost, J. W., Berry, A., Disbrow, G. L., Staversky, R. J., and Lievense, J. C. (1992) Biocatalytic Synthesis of Aromatics from D-Glucose—the Role of Transketolase, Journal of the American Chemical Society 114, 3956-3962.
Draths, K. M., and Frost, J. W. (1995) Environmentally Compatible Synthesis of Catechol from D-Glucose, Journal of the American Chemical Society 117, 2395-2400.
Elsemore, D. A., and Ornston, L. N. (1995) Unusual ancestry of dehydratases associated with quinate catabolism in Acinetobacter calcoaceticus, J Bacteriol 177, 5971-5978.
Escalante, A., Calderon, R., Valdiva, A., de And a, R., Hernandez, G., Ramirez, O. T., Gosset, G. and Boliver, F. (2010) Metabolic engineering for the production of shikimic acid in an evolved Escherichia coli strain lacking the phosphoenolpyrvate: carbohydrate phosphotransferase system. Microbial Cell Factories 9, 21-33.
Flores, N., Xiao, J., Berry, A., Bolivar, F. and Valle, F. (1996) Pathway engineering for the production of aromatic compounds in Escherichia coli. Nature Biotechn. 14, 620-623.
Fox, D. T., Hotta, K., Kim, C. Y., and Koppisch, A. T. (2008) The missing link in petrobactin biosynthesis: asbF encodes a (−)-3-dehydroshikimate dehydratase, Biochemistry 47, 12251-12253.
Ger, Y., Chen, S., Chiang, H., and Shiuan, D. (1994) A Single Ser-180 Mutation Desensitizes Feedback Inhibition of the Phyenylalanine-Sensitive 3-Deoxy-D-Arabino-Hepulosonate 7-Phosphate (DAHP) Synthetase in Escherichia coli, J Biochem 116, 986-990.
Grant, D. J., and Patel, J. C. (1969) The non-oxidative decarboxylation of p-hydroxybenzoic acid, gentisic acid, protocatechuic acid and gallic acid by Klebsiella aerogenes (Aerobacter aerogenes), Antonie Van Leeuwenhoek 35, 325-343.
Hansen, E. H., Moller, B. L., Kock, G. R., Bunner, C. M., Kristensen, C., Jensen, O. R., Okkels, F. T., Olsen, C. E., Motawia, M. S., and Hansen, J. (2009) De novo biosynthesis of vanillin in fission yeast (Schizosaccharomyces pombe) and baker's yeast (Saccharomyces cerevisiae), Appl Environ Microbiol 75, 2765-2774.
Hu, C., Jiang, P., Xu, J., Wu, Y., and Huang, W. (2003) Mutation analysis of the feedback inhibition site of phenylalanine-sensitive 3-deoxy-D-arabino-heptulosonate 7-phosphate synthase of Escherichia coli, J Basic Microbiol 43, 399-406.
Iwagami, S. G., Yang, K., and Davies, J. (2000) Characterization of the protocatechuic acid catabolic gene cluster from Streptomyces sp. strain 2065, Appl Environ Microbiol 66, 1499-1508.
Jantama, K., Haupt, M. J., Svoronos, S. A., Zhang, X., Moore, J. C., Shanmugam, K. T., and Ingram, L. O. (2008a) Combining metabolic engineering and metabolic evolution to develop nonrecombinant strains of Escherichia coli C that produce succinate and malate, Biotechnol Bioeng 99, 1140-1153.
Jantama, K., Zhang, X., Moore, J. C., Shanmugam, K. T., Svoronos, S. A., and Ingram, L. O. (2008b) Eliminating side products and increasing succinate yields in engineered strains of Escherichia coli C, Biotechnol Bioeng 101, 881-893.
Kaneko, A., Ishii, Y., and Kirimura, K. (2011) High-yield production of cis,cis-muconic acid from catechol in aqueous solution by biocatalyst. Chem. Lett. 40, 381-383.
Kikuchi, Y., Tsujimoto, K., and Kurahashi, O. (1997) Mutational analysis of the feedback sites of phenylalanine-sensitive 3-deoxy-D-arabino-heptulosonate-7-phosphate synthase of Escherichia coli, Appl Environ Microbiol 63, 761-762.
Kojima, Y., Fujisawa, H., Nakazawa, A., Nakazawa, T., Kanetsuna, F., Taniuchi, H., Nozaki, M., and Hayaishi, O. (1967) Studies on pyrocatechase. I. Purification and spectral properties, J Biol Chem 242, 3270-3278.
Kramer, M., Bongaerts, J., Bovenberg, R., Kremer, S., Muller, U., Orf, S., Wubbolts, M. and Raeven, L. (2003) Metabolic engineering for microbial production of shikimic acid. Metabol. Eng. 5, 277-283.
Lerner, C. G., and Inouye, M. (1990) Low copy number plasmids for regulated low-level expression of cloned genes in Escherichia coli with blue/white insert screening capability, Nucleic Acids Res 18, 4631.
Li, K. and Frost, J. W. (1999) Microbial synthesis of 3-dehydroshikimic acid: A comparative analysis of D-xylose, L-arabinose, and D-glucose carbon sources. Biotechnol. Prog. 15, 876-883.
Lu, J. L., and Liao, J. C. (1997) Metabolic engineering and control analysis for production of aromatics: Role of transaldolase, Biotechnol Bioeng 53, 132-138.
Lu, J., Tang, J., Liu, Y., Zhu, X. (2012) Combinatorial modulation of galP and glk gene expression for improves alternative glucose utilization. Appl. Microbiol. Biotechnol. 93, 2455-2462
Lutke-Eversloh, T., and Stephanopoulos, G. (2007) L-tyrosine production by deregulated strains of Escherichia coli, Appl Microbiol Biotechnol 75, 103-110.
Mizuno, S., Yoshikawa, N., Seki, M., Mikawa, T., and Imada, Y. (1988) Microbial production of cis,cis-muconic acid from benzoic acid. Appl Microbiol Biotechnol. 28, 20-25.
Nakazawa, A., Kojima, Y., and Taniuchi, H. (1967) Purification and properties of pyrocatechase from Pseudomonas fluorescens, Biochim Biophys Acta 147, 189-199.
Neidhardt, F. C., and Curtiss, R. (1996) Escherichia coli and Salmonella: cellular and molecular biology, 2nd ed., ASM Press, Washington, D.C.
Neidle, E. L., and Ornston, L. N. (1986) Cloning and expression of Acinetobacter calcoaceticus catechol 1,2-dioxygenase structural gene catA in Escherichia coli, J Bacteriol 168, 815-820.
Niu, W., Draths, K. M., and Frost, J. W. (2002) Benzene-free synthesis of adipic acid, Biotechnol Prog 18, 201-211.
Parker, C., Barnell, W. O., Snoep, J. L., Ingram, L. O., and Conway, T. (1995) Characterization of the Zymomonas mobilis glucose facilitator gene product (glf) in recombinant Escherichia coli: examination of transport mechanism, kinetics and the role of glucokinase in glucose transport, Mol Microbiol 15, 795-802.
Parsek, M. R., Shinabarger, D. L., Rithmel, R. K. and Chakrabarty, A. M. (1992) Roles of CatR and cis,cis-Muconate in activation of the catBC operson, which is involved in benzoate degradationin Pseudomonas putida. J. Bacteriol. 174, 7798-7806.
Patnaik, R. and Liao, J. C. (1994) Engineering of Escherichia coli central metabolism for aromatic metabolite with near theoretical yield. App. Env. Microbiol. 60, 3903-3908.
Pfleger, B. F., Kim, Y., Nusca, T. D., Maltseva, N., Lee, J. Y., Rath, C. M., Scaglione, J. B., Janes, B. K., Anderson, E. C., Bergman, N. H., Hanna, P. C., Joachimiak, A., and Sherman, D. H. (2008) Structural and functional analysis of AsbF: origin of the stealth 3,4-dihydroxybenzoic acid subunit for petrobactin biosynthesis, Proc Natl Acad Sci USA 105, 17133-17138.
Perez-Pantoja, D., De la Iglesia, R., Pieper, D. H., and Gonzalez, B. (2008) Metabolic reconstruction of aromatic compounds degradation from the genome of the amazing pollutant-degrading bacterium Cupriavidus necator JMP134, FEMS Microbiol Rev 32, 736-794.
Perez-Pantoja, D., Donoso, R., Agullo, L., Cordova, M., Seeger, M., Pieper, D. H., and Gonzalez, B. (2011) Genomic analysis of the potential for aromatic compounds biodegradation in Burkholderiales, Environ Microbiol.
Pittard, J. and Wallace, B. J. (1966) Distribution and function of genes concerned with aromatic biosynthesis in Escherichia coli. J. Bacteriol. 91, 1494-1508.
Polen, T., Spelberg, M. and Bott, M. (2012) toward bitechnological produciton of adipic acid and precursors from biorenewables, J. Biotechnol, http://dx.doi.org/10.1016/j.biotec.2012-07.008.
Rutledge, B. J. (1984) Molecular characterization of the qa-4 gene of Neurospora crassa, Gene 32, 275-287.
Schirmer, F., and Hillen, W. (1998) The Acinetobacter calcoaceticus NCIB8250 mop operon mRNA is differentially degraded, resulting in a higher level of the 3′ CatA-encoding segment than of the 5′ phenolhydroxylase-encoding portion, Mol Gen Genet 257, 330-337.
Shumilin, I. A., Kretsinger, R. H., and Bauerle, R. H. (1999) Crystal structure of phenylalanine-regulated 3-deoxy-D-arabino-heptulosonate-7-phosphate synthase from Escherichia coli, Structure 7, 865-875.
Shumilin, I. A., Zhao, C., Bauerle, R., and Kretsinger, R. H. (2002) Allosteric inhibition of 3-deoxy-D-arabino-heptulosonate-7-phosphate synthase alters the coordination of both substrates, J Mol Biol 320, 1147-1156.
Shumilin, I. A., Bauerle, R., Wu, J., Woodard, R. W., and Kretsinger, R. H. (2004) Crystal structure of the reaction complex of 3-deoxy-D-arabino-heptulosonate-7-phosphate synthase from Thermotoga maritima refines the catalytic mechanism and indicates a new mechanism of allosteric regulation, J Mol Biol 341, 455-466.
Shumkova, E. S., Solyanikova, I. P., Plotnikova, E. G. and Golovleva, L. A. (2009) Phenol degrdation by Rhodococcus opacus Strain 1G. App. Biocehm. Microbiol. 45, 43-49.
Sietmann, R., Uebe, R., Boer, E., Bode, R., Kunze, G., and Schauer, F. (2010) Novel metabolic routes during the oxidation of hydroxylated aromatic acids by the yeast Arxula adeninivorans, J Appl Microbiol 108, 789-799.
Smith, M. R. and Ratledge, C. (1989) Quantitative biotransformation of catechol to cis,cis-muconate. Biotech. Lett. 11, 105-110.
Snoep, J. L., Arfman, N., Yomano, L. P., Fliege, R. K., Conway, T., and Ingram, L. O. (1994) Reconstruction of glucose uptake and phosphorylation in a glucose-negative mutant of Escherichia coli by using Zymomonas mobilis genes encoding the glucose facilitator protein and glucokinase, J Bacteriol 176, 2133-2135.
Sprenger, G. A. (1995) Genetics of pentose-phosphate pathway enzymes of Escherichia coli K-12, Arch Microbiol 164, 324-330.
Sprenger, G. A., Schorken, U., Sprenger, G., and Sahm, H. (1995a) Transketolase A of Escherichia coli K12. Purification and properties of the enzyme from recombinant strains, Eur J Biochem 230, 525-532.
Sprenger, G. A., Schorken, U., Sprenger, G., and Sahm, H. (1995b) Transaldolase B of Escherichia coli K-12: cloning of its gene, talB, and characterization of the enzyme from recombinant strains, J Bacteriol 177, 5930-5936.
Stroman, P., Reinert, W. R., and Giles, N. H. (1978) Purification and characterization of 3-dehydroshikimate dehydratase, an enzyme in the inducible quinic acid catabolic pathway of Neurospora crassa, J Biol Chem 253, 4593-4598.
Tang, J., Zhu, X., Lu, J. and Liu, P. (2012) Recruiting alternative glucose utilization pathways for improving succinate production. App Microbiol Biotechnol DOI 10, 1007/s00253-012-434.1
Tateoka, T., and Yasuda, I. (1995) 3-Dehydroshikimate dehydratase in mung bean cultured cells, Plant Cell Reports 15, 212-217.
Weaver, L. M., and Herrmann, K. M. (1990) Cloning of an aroF allele encoding a tyrosine-insensitive 3-deoxy-D-arabino-heptulosonate 7-phosphate synthase, J Bacteriol 172, 6581-6584.
Weber, C., Bruckner, C., Weinreb, S., Lehr, C., Essl, C. and Bole, E. (2012) Biosynthesis of cis,cis-muconic acid and its aromatic precursors catechol and proteocatechuic acid, from renewable feedstocks by Saccharomyces cerevisiae, App Environ Microbiol. 78, 8421-8430.
Wheeler, K. A., Lamb, H. K., and Hawkins, A. R. (1996) Control of metabolic flux through the quinate pathway in Aspergillus nidulans, Biochem J 315 (Pt 1), 195-205.
Wu, C-M., Wu, C-C., Su, C-C., Lee, S-N., Lee, Y-A. and Wu, J-Y. (2006) Microbial synthesis of cis,cis-muconic acid form benzoate by Sphingobacterium sp. Mutants. Biochem. Eng. J. 29, 35-40.
Xie, N., Tang, H., Feng, J., Tao, F., Ma, C. and Xu, P. (2009) Characterization of benzoate degradation by newly isolated bacterium Pseudomonas sp. XP-M2. Biochem. Eng. J. 46, 79-82.
Yi, J., Draths, K. M., Li, K. and Frost, J. W. (2003) Altered Glucose Transport and Shikimate Pathway Product Yields in E. coli. Biotechnol. Prog. 2003, 19, 1450-1459
Yoshikawa, N., Mizuno, S., Ohta, K., and Suzuki, M. (1990) Microbial production of cis,cis-muconic acid. J. Biotechnol. 14, 203-210

What is claimed is:

1. A genetically engineered microorganism that produces cis,cis-muconic acid starting from a non-aromatic carbon source, in which all genes coding for proteins functioning in a muconic acid pathway are integrated into the chromosomal DNA of said microorganism.

2. A genetically engineered microorganism of claim 1 wherein said genes coding for proteins functioning in a muconic acid pathway are selected from a group consisting of aroZ, qa-4, asbF, quiC, aroY, and catAX.

3. A genetically engineered microorganism organism of claim 1 comprising a gene that encodes for a QuiC enzyme of a bacterium of the genus Acinetobacter, or a homolog of said QuiC enzyme.

4. A genetically engineered microorganism of claim 1 comprising one or more exogenous genes coding for proteins functioning in a shikimic acid pathway.

5. A genetically engineered microorganism of claim 4, wherein one or more of said exogenous genes coding for proteins functioning in a shikimic acid pathway are integrated into the chromosomal DNA of said organism.

6. A genetically engineered microorganism of claim 4 wherein said exogenous genes coding for proteins functioning in a shikimic acid pathway are chosen from a group consisting of aroB, aroD, aroF, aroG, aroH, tktA, talB, rpe, and rpi.

7. A genetically engineered organism of claim 4 wherein none of the said genes coding for proteins functioning in muconic acid pathway or shikimic acid pathway are expressed from a promoter that requires a fed chemical for induction.

8. A genetically engineered host bacterium of claim 1 wherein the activity of a negative regulator protein of aromatic amino acid biosynthesis substantially reduced or eliminated.

9. A genetically engineered microorganism of claim 8, wherein the negative regulator protein of aromatic amino acid biosynthesis is a TyrR protein or a homolog of a TyrR protein.

10. A genetically engineered microorganism of claim 8, wherein the gene that codes for a negative regulator protein of aromatic amino acid biosynthesis has been mutated.

11. A genetically engineered organism of claim 1 comprising an aroE gene that encodes a leaky AroE enzyme.

12. A genetically engineered microorganism organism of claim 1 in which at least one enzyme active in the shikimic acid pathway is substantially resistant to inhibition by a metabolite of said microorganism.

13. A genetically engineered organism of claim 12 comprising an aroG* gene that encodes a DAHP synthase enzyme that is substantially resistant to inhibition by phenylalanine.

14. A genetically engineered organism of claim 12 comprising an aroG* gene that encodes a DAHP synthase enzyme that is selected from the set consisting of aroG*20-893, aroG*20-897 aroG*20-899, aroG*20-901, aroG*111, aroG*211, aroG*212, aroG*311, aroG*312, aroG*411, aroG*412, and aroG*511.

15. A genetically engineered microorganism of claim 1 wherein said microorganism is a bacterium.

16. A genetically engineered microorganism of claim 1 wherein said microorganism is derived from Escherichia coli C.

17. A genetically engineered bacterium that is deficient in a PEP dependent phosphotransferase system, deficient in a GalP protein based system for glucose import, and comprises an exogenous gene that encodes a functional glucose-facilitated diffusion protein.

18. A genetically engineered bacterium as in claim 17, wherein the functional glucose-facilitated diffusion protein is Glf protein coded by glf gene.

19. A genetically engineered bacterium as in claim 17, further comprising an exogenous gene that encodes a glucokinase.

20. A genetically engineered bacterium as in claim 17, further comprising one or more exogenous genes encoding a protein that functions in a muconic acid pathway that produces cis,cis-muconic acid starting from a non-aromatic carbon source.

21. A genetically engineered bacterium of claim 20, wherein said genes coding for proteins functioning in a muconic acid pathway are selected from a group consisting of aroZ, qa-4, asbF, quiC, aroY, and catAX.

22. A genetically engineered bacterium as in claim 17, further comprising one or more chromosomally integrated exogenous genes coding for proteins functioning in a shikimic acid pathway,

23. A genetically engineered bacterium as in claim 22 wherein said exogenous genes coding for proteins functioning in a shikimic acid pathway are chosen from a group consisting of aroB, aroD, aroF, aroG, aroH, tktA, talB, rpe, and rpi.

24. A genetically engineered bacterium of claim 17 wherein the activity of a negative regulator protein of aromatic amino acid biosynthesis is substantially reduced or eliminated.

25. A genetically engineered bacterium of claim 17, wherein the negative regulator protein of aromatic amino acid biosynthesis is a TyrR protein or a homolog of a TyrR protein.

26. A genetically engineered bacterium of claim 17, wherein the gene that codes for a negative regulator protein of aromatic amino acid biosynthesis has been deleted or mutated.

27. A genetically engineered bacterium of claim 17, wherein at least one enzyme active in the shikimic acid pathway is substantially resistant to inhibition by a metabolite of said microorganism.

28. A genetically engineered bacterium of claim 17, wherein said microorganism is derived from Escherichia coli C.

29. A genetically engineered microorganism that produces cis,cis-muconic acid starting from a non-aromatic carbon source wherein exogenous genes coding for proteins functioning in muconic acid pathway and shikimic acid pathway are not expressed from a promoter that requires a fed chemical for induction.

30. A genetically engineered microorganism as in claim 29, wherein said microorganism is a bacterium.

31. A genetically engineered microorganism as in claim 29, wherein said microorganism is Escherichia coli.

32. A genetically engineered microorganism that produces cis,cis-muconic acid starting from a non-aromatic carbon source comprising an aroE gene that encodes a leaky AroE enzyme.

33. A genetically engineered microorganism as in claim 32, wherein said microorganism is a bacterium.

34. A genetically engineered microorganism as in claim 32, wherein said microorganism is Escherichia coli.
